(19) United States
(12) Patent Application Publication
(10) Pub. No.: US 2023/0064780 A1
(43) Pub. Date: Mar. 2, 2023

(54) MYCOSPORINE-LIKE AMINO ACID-PRODUCING MICROORGANISM AND METHOD FOR PRODUCTION OF MYCOSPORINE-LIKE AMINO ACIDS BY USING SAME

(71) Applicants: Cj CheilJedang Corporation, Seoul (KR); Seoul National R&DB Foundation, Seoul (KR)

(72) Inventors: Ji-Sook HAHN, Seoul (KR); Seong-Hee PARK, Suwon-si (KR); Kyusung LEE, Suwon-si (KR); Jae Woo JANG, Suwon-si (KR)

(21) Appl. No.: 17/788,058

(22) PCT Filed: Dec. 24, 2020
(86) PCT No.: PCT/KR2020/019094

§ 371 Date: Jun. 22, 2022

(30) Foreign Application Priority Data
Dec. 24, 2019 (KR) 10-2019-0174548

(51) Int. Cl. C12P 13/00; C12P 13/04; C12N 15/52

(52) U.S. Cl. CPC C12P 13/005; C12P 13/04; C12Y 207/01017; C12Y 503/01005; C12Y 101/01009; C12Y 202/01001; C12N 15/52

(57) ABSTRACT

Provided are a mycosporine-like amino acid-producing microorganism and a method for production of mycosporine-like amino acids by using same. The microorganism can produce mycosporine-like amino acids from xylose.

### CROSS-REFERENCE TO RELATED APPLICATION(S)

This application claims the benefit of and priority of Korean Patent Application No. 10-2019-0174548 filed on Dec. 24, 2019 with the Korean Intellectual Property Office, the content of which is incorporated herein by reference in its entirety.

### TECHNICAL FIELD

The present disclosure relates to a microorganism for producing mycosporine-like amino acid and a method for preparing mycosporine-like amino acid using the same.

### BACKGROUND ART

Various chemical and physical ultraviolet blocking substances are used to protect a skin from ultraviolet rays. Particularly, oxybenzone, zinc oxide (ZnO), and titanium dioxide (TiO₂) are ultraviolet blocking substances that are widely used as cosmetic additives, but causes dermatitis or has negative effects such as environmental pollutions, and therefore, it is necessary to develop safer bio-based ultraviolet blocking substances.

Mycosporine and mycosporine-like amino acids (MAAs) are natural ultraviolet blocking substances produced by prokaryotic and eukaryotic microorganisms such as microalgae, fungi, and algae that exist in ecosystems. Particularly, the mycosporine-like amino acid is a form in which a nitrogen compound is bound to a cyclohexanamine core structure, and more than 30 various mycosporine-like amino acids have been reported depending on the type of the bound compound.

A typical example of mycosporine-like amino acids may be shinorine. Shinorine is a compound having glycine and serine substituents, which is in the spotlight as an effective ultraviolet blocking agent. Shinorine has a very high extinction coefficient (ε=28,100-50,000 M⁻¹ cm⁻¹), which is three times higher than that of oxybenzone (ε=14,295) known as an effective sunscreen substance. Therefore, shinorine can be said to be a very high-efficiency ultraviolet blocking substance. In addition, shinorine is an effective material that can specifically block ultraviolet rays from UV-A, which is ultraviolet rays that most commonly reach the earth. Mycosporine-like amino acids, including shinorine, are naturally produced by microorganisms such as microalgae, but there is a problem that the amount is very small, and the conditions for culturing microalgae and separating, extracting, and purifying mycosporine-like amino acids are complicated, and therefore, it is difficult to mass-produce mycosporine-like amino acids.

Therefore, there is a need to develop a new strain having excellent mycosporine-like amino acid production efficiency.

### DETAILED DESCRIPTION OF THE INVENTION

### Technical Problem

One embodiment provides a microorganism comprising (a) xylose assimilation enzyme, and/or agene encoding the same, and (b) mycosporine-like amino acid (MAA) biosynthesis enzyme, and/or a gene encoding the same.

The microorganism may be further one in which a pentose phosphate pathway is enhanced. The xylose assimilation enzyme may comprise a conversion enzyme of xylose to xylulose, a xylulose phosphorylase, or a combination thereof. The xylose assimilation enzyme, mycosporine-like amino acid biosynthesis enzyme, or both may be a microbial endogenous protein and/or exogenous protein. The enhancement of the pentose phosphate pathway may mean that the proteins involved in the pentose phosphate pathway are activated as compared with non-mutant microorganisms. The microorganism may be for producing mycosporine-like amino acids and/or for use in producing mycosporine-like amino acids.

Another embodiment provides a composition for the production of a mycosporine-like amino acid comprising (i) a xylose assimilation enzyme, agene encoding the same, or a recombinant vector containing the same; (ii) a mycosporine-like amino acid biosynthesis enzyme, a gene encoding the same, or a recombinant vector containing the same; and/or (iii) a microorganism comprising the (i), (ii), or a combination thereof. The composition or the microorganisms comprised in the composition may further comprise a component for enhancing the pentose phosphate pathway.

Another embodiment provides a method for producing a microorganism that produces mycosporine-like amino acids, the method comprising the steps of: (1) introducing a xylose assimilation enzyme, a gene encoding the same, or a recombinant vector containing the same into a microorganism; (2) introducing a mycosporine-like amino acid biosynthesis enzyme, a gene encoding the same, or a recombinant vector containing the same into a microorganism; or a combination of the steps (1) and (2). The method may further comprise the step (3) of enhancing the pentose phosphate pathway.

Yet another embodiment provides a method for producing mycosporine-like amino acids, comprising a step of culturing the microorganism.

### Technical Solution

The present disclosure proposes that when a microorganism containing mycosporine-like amino acid biosynthesis enzyme, and/or a gene encoding the same contains a xylose assimilation enzyme, and/or a gene encoding the same, and/or is one in which a pentose phosphate pathway is enhanced, the production efficiency of mycosporine-like amino acids is enhanced.

As used herein, the term ‘comprising or consisting of an amino acid sequence or a nucleic acid sequence’ may be used to mean a case that contains or consists essentially of a predetermined sequence described.

Further, a protein (e.g., enzyme) or gene comprising a specific amino acid sequence or nucleic acid sequence described herein (1) may be construed as a protein or gene comprising a sequence 100% identical to the specific amino acid sequence or nucleic acid sequence, or (2) may be construed as any protein or gene comprising a sequence having at least 80%, at least 85%, at least 90%, at least 95%, at least 96%, at least 97%, at least 98%, or at least 99% sequence identity thereto. The protein or gene comprising a sequence having the sequence identity of (2) may be a protein maintaining the function (e.g., original enzyme activity) of the original protein (containing sequences with 100% identity) or agene encoding the same. Maintaining the function of the protein may refer to maintaining at least 80%, at least 85%, at least 90%, at least 95%, at least 96%, at least 97%, at least 98%, or at least 99% of the enzymatic activity of the original protein (containing sequences with 100% identity), for example, as measured in microorganisms and/or in vitro.

As used herein, the terms “mutant microorganism”, “mutant strain”, “recombinant microorganism”, “recombinant strain”, “genetically modified microorganism” or “genetically modified strain” may refer to a microorganism comprising modification (nucleic acid deletion, addition, substitution, etc.) of an endogenous gene and/or expression regulatory sequence, insertion of a foreign gene into the genome, and/or introduction of a plasmid containing the endogenous gene. In one specific embodiment, the microorganism may be non-naturally occurred by conventional recombinant technology and/or genome or gene editing technology.

As used herein, “strain prior to modification” or “microorganism prior to modification” does not exclude strains containing mutations that may occur naturally in microorganisms, and may refer to a naturally occurring strain itself or a strain before change of a character through genetic modification due to natural or artificial factors. In the present disclosure, the change in the character may be the introduction of a specific protein (or gene) and/or enhancement of the activity of the specific protein. The “strain prior to modification” or “microorganism prior to modification” may be used interchangeably with “parent strain”, “unmutated strain”, “unmodified strain”, “unmutated microorganism”, “unmodified microorganism” or “reference microorganism”.

As used herein, the term “vector” collectively refers to all types of nucleic acid sequence carrier constructs that are used to deliver agene or polynucleotide fragment to a host cell, and unless otherwise specified, it can mean that the carried nucleic acid sequence is inserted and expressed into a host cell genome, and/or expressed independently.

As used herein, the term “gene encoding a protein” is a nucleic acid molecule that comprises a nucleic acid sequence corresponding to (encoding) the amino acid sequence of the protein, and the nucleic acid sequence may comprise various modifications of the coding region within the range that does not change the amino acid sequence and/or function of the protein expressed from the coding region, taking into account the condones preferred in the microorganism for expressing the protein due to codon degeneracy.

One embodiment provided herein provide mutant microorganisms with the following characteristics:

A mutant microorganism comprising (a) a xylose assimilation enzyme, and/or a gene encoding the same; and (b) a mycosporine-like amino acid biosynthesis enzyme, and/or a gene encoding the same is provided.

The mutant microorganism may be one in which a pentose phosphate pathway is further enhanced. The xylose assimilation enzyme may comprise a conversion enzyme of xylose to xylulose, a xylulose phosphorylase, or a combination thereof. The xylose assimilation enzyme, mycosporine-like amino acid biosynthesis enzyme, or both may be the mutant microorganism endogenous proteins and/or exogenous proteins. The enhancement of the pentose phosphate pathway may mean that the proteins involved in the pentose phosphate pathway are activated as compared with non-mutant microorganisms.

The enhancement of the pentose phosphate pathway can be achieved through an increase in the transketolase activity to produce sedoheptulose 7-phosphate in the pentose phosphate pathway, an increase in the protein activity that regulates NADPH production, and a reduction in the transaldolase activity involved in the conversion reaction between sedoheptulose 7-phosphate and glyceraldehyde 3-phosphate.

The mutant microorganism may have the ability to produce mycosporine-like amino acids. The mutant microorganism may be one that produces mycosporine-like amino acids. The mutant microorganism may be one capable of using xylose as a carbon source. The mutant microorganism may produce mycosporine-like amino acids from xylose.

Another embodiment provides a composition for producing mycosporine-like amino acids comprising (i) a xylose assimilation enzyme, a gene encoding the same, or a recombinant vector containing the same; (ii) a mycosporine-like amino acid biosynthesis enzyme, a gene encoding the same, or a recombinant vector containing the same; and/or (iii) a mutant microorganism comprising (i), (ii), or a combination thereof. The composition or the mutant microorganism comprised in the composition may further comprise a component for enhancing the pentose phosphate pathway.

The xylose assimilation enzyme, a gene encoding the same, and/or a mycosporine-like amino acid biosynthesis enzyme, and a gene encoding the same may be the mutant microorganism endogenous protein (gene) and/or exogenous protein (gene).

In one specific embodiment, the gene encoding the xylose assimilation enzyme is a foreign gene, which may be a gene encoding a conversion enzyme of xylose to xylulose, and/or a xylulose phosphorylase.

In another embodiment, the enhancement of the pentose phosphate pathway can be achieved through at least one selected from an increase in the transketolase activity to produce sedoheptulose 7-phosphate in the pentose phosphate pathway, an increase in the protein activity that regulates NADPH production, and a reduction in the transaldolase activity involved in the conversion reaction between sedoheptulose 7-phosphate and glyceraldehyde 3-phosphate.

The reduction in the transaldolase activity can be performed by introducing a repressor that inhibits the expression of a gene encoding transaldolase, weakening the expression regulatory sequence (e.g., replacing the expression regulatory sequence with a weaker expression regulatory sequence than before replacement), or by introducing a protein and/or polynucleotide for deletion and/or substitution of the gene. In one specific embodiment, protein and/or polynucleotide for replacement of the expression regulatory sequence of the a transaldolase-encoding gene, or deletion or replacement of all or part of the gene may be at least one selected from the group consisting of RNA-guided endonuclease system (e.g., (a) an RNA-guided endonuclease (e.g., Cas9 protein, etc.), a gene encoding the same, or a vector containing the gene; and (b) guide RNA (e.g., single guide RNA (sgRNA), etc.), a mixture containing the encoding DNA thereof, or a vector containing the DNA (e.g., a mixture of RNA-guided endonuclease protein and guide RNA, etc.), a complex (e.g., ribonucleic acid protein (RNP) in which RNA-guided endonuclease protein and guide RNA are fused, recombinant vector (e.g., vector containing RNA-guided endonuclease encoding gene and guide RNA encoding DNA, etc.), and the like.

The composition can produce mycosporine-like amino acids from xylose.

Another embodiment provides a method for producing a mutant microorganism that produces mycosporine-like amino acids, the method comprising the steps of: (1) introducing a xylose assimilation enzyme, agene encoding the same, or a recombinant vector containing the same into a microorganism; (2) introducing a mycosporine-like amino acid biosynthesis enzyme, a gene encoding the same, or a recombinant vector containing the same into a microorganism; or a combination of the steps (1) and (2). The step (1) and the step (2) may be performed simultaneously or may be sequentially performed regardless of the order (that is, performing step (2) after performing step (1), or performing step (2) after performing step (1))

The method may further comprise a step (3) of enhancing the pentose phosphate pathway. In this case, the steps (1) to (3) may be performed simultaneously or performed irrespective of the order.

Another embodiment provides a method for producing mycosporine-like amino acids, comprising a step of culturing the mutant microorganism. The production method may further comprise, after the culturing, recovering mycosporine-like amino acids from the cultured microorganism, medium, or a combination thereof.

Hereinafter, the present disclosure will be described in more detail:

Xylose Assimilation Enzyme and Gene Encoding the Same

The xylose assimilation enzyme may comprise one or more of the enzymes involved in a series of metabolic (fermentation) pathways that convert xylose into a form that can be introduced into the pentose phosphate pathway, and the gene encoding the same is a gene encoding the enzyme, and may be designed so as to comprise codons optimized for the cell to be transduced. In one specific embodiment, the xylose assimilation enzyme and/or a gene encoding the same may be derived from a heterologous to the mutant microorganism, that is, from a microorganism of a different species from the mutant microorganism.

In one embodiment, the xylose assimilation enzyme may comprise:

a conversion enzyme of xylose to xylulose, for example, xylose isomerase (XI), xylose reductase (XR), xylitol dehydrogenase (XDH) or a combination thereof; xylulose phosphorylase such as xylulokinase (XK); or a combination thereof.

In one embodiment, the mutant microorganism may be one into which a gene encoding a xylose assimilation enzyme derived from allogeneic or heterologous species is introduced.

The xylose isomerase (XI) is an enzyme that catalyzes the interconversion between D-xylose and D-xylulose, which is an enzyme involved in consuming most of the xylose in microorganisms (e.g., bacteria). In one specific embodiment, the xylose isomerase may be derived from at least one selected from the group consisting of Pichia stipitis, Paraburkhoderia sacchari, Actinomyces olivocinereus, Actinoplanes missouriensis, Aerobacter levanicum, Bacillus coagulans, Bacillus sp., Bifdobacterium adolescents, Bacteroides thetaiotaomicron, Clostridium cellulovorans, Clostridium phytofermetans, Lactobacillus brevis, Lactococcus lactis, Orpinomyces sp., Piromyces sp., Thermus thermophiles, and Vibrio sp., and the like.

The xylose reductase (XR) and the xylitol dehydrogenase (XDH) is an enzyme involved in the continuous conversion from xylose to xylitol and from xylitol to xylulose. In one embodiment, the xylose reductase (XR) and the xylitol dehydrogenase (XDH) may be derived from at least one selected from the group consisting of genus Pichia (e.g., Pichia stipitis, Pichia segobiensis, etc.), Aspergillus carbonarius, genus Candida (e.g., Candida boidinii, Candida diddensiae, Candida intermedia, Candida tenuis, Candida shehatae, etc.), Kluyveromyces marxianus, Neurospora crassa, Ogataea siamensis, Trichoderma reesei, Zymomonas mobilis, Pachysolen tannophilus, Odontotaenius disjunctus, Hansenula polymorpha, and the like.

In one embodiment, for the conversion of xylose to xylulose, XR/XDH having relatively well maintained enzymatic activity upon introduction into a heterologous cell can be used.

The xylulokinase (XK) is an enzyme involved in converting xylulose into xylulose-5-phosphate (X5P). In one embodiment, the xylulokinase (XK) may be derived from at least one microorganism selected from the group consisting of Pichia stipitis, Arabidopsis thaliana, Bacillus coagulans, Klebsiella pneumonia, Kluyveromyces marxianus, Hansenula polymorpha, Saccharomyces cerevisiae, Thermotoga maritime, Trichoderma reesei, and Zymomonas mobilis, and the like

In one embodiment, the xylose assimilation enzyme may comprise xylose reductase (XR), xylitol dehydrogenase (XDH), and xylulokinase (XK) derived from Pichia stipitis.

The metabolic pathway in which the xylose assimilation enzyme is involved is exemplarily shown in FIG. 1a.

The product of the metabolic pathway (e.g., X5P) in which the xylose assimilation enzyme is involved can increase the production of an intermediate product of the pentose phosphate pathway (e.g., sedoheptulose 7-phosphate (S7P)).

The xylose assimilation enzyme and the gene encoding the same are exemplified in Table 1 below:

TABLE 11

| | | GenBank | | |
|---|---|---|---|---|
| | | Accession | Amino acid | |
| Enzyme | Origin | no. | sequence (N→C) | Nucleic acid sequence (5′→3′) |
| XXR | Pichia | XM_ | MPSIKLNSGYD | ATGCCTTCTATTAAGTTGAACTCTGGTTA |
| | stipitis | 001385144.1 | MPAVGFGCW | CGACATGCCAGCCGTCGGTTTCGGCTGTT |
| | | | KVDVDTCSEQI | GGAAAGTCGACGTCGACACCTGTTCTGA |
| | | | YRAIKTGYRLF | ACAGATCTACCGTGCTATCAAGACCGGTT |
| | | | DGAEDYANEK | ACAGATTGTTCGACGGTGCCGAAGATTA |
| | | | LVGAGVKKAI | CGCCAACGAAAAGTTAGTTGGTGCCGGT |
| | | | DEGIVKREDLF | GTCAAGAAGGCCATTGACGAAGGTATCG |
| | | | LTSKLWNNYH | TCAAGCGTGAAGACTTGTTCCTTACCTCC |
| | | | HPDNVEKALN | AAGTTGTGGAACAACTACCACCACCCAG |
| | | | RTLSDLQVDY | ACAACGTCGAAAAGGCCTTGAACAGAAC |
| | | | VDLFLIHFPVT | CCTTTCTGACTTGCAAGTTGACTACGTTG |
| | | | FKFVPLEEKYP | ACTTGTTCTTGATCCACTTCCCAGTCACCT |
| | | | PGFYCGKGDN | TCAAGTTCGTTCCATTAGAAGAAAAGTAC |
| | | | FDYEDVPILET | CCACCAGGATTCTACTGTGGTAAGGGTG |
| | | | WKALEKLVK | ACAACTTCGACTACGAAGATGTTCCAATT |
| | | | AGKIRSIGVSN | TTAGAGACCTGGAAGGCTCTTGAAAAGT |
| | | | FPGALLLDLLR | TGGTCAAGGCCGGTAAGATCAGATCTAT |
| | | | GATIKPSVLQV | CGGTGTTTCTAACTTCCCAGGTGCTTTGC |
| | | | EHHPYLQQPR | TCTTGGACTTGTTGAGAGGTGCTACCATC |
| | | | LIEFAQSRGIA | AAGCCATCTGTCTTGCAAGTTGAACACCA |
| | | | VTAYSSFGPQS | CCCATACTTGCAACAACCAAGATTGATCG |
| | | | FVELNQGRAL | AATTCGCTCAATCCCGTGGTATTGCTGTC |
| | | | NTSPLFENETI | ACCGCTTACTCTTCGTTCGGTCCTCAATCT |
| | | | KAIAAKHGKS | TTCGTTGAATTGAACCAAGGTAGAGCTTT |
| | | | PAQVLLRWSS | GAACACTTCTCCATTGTTCGAGAACGAAA |
| | | | QRGIAIIPKSNT | CTATCAAGGCTATCGCTGCTAAGCACGGT |
| | | | VPRLLENKDV | AAGTCTCCAGCTCAAGTCTTGTTGAGATG |
| | | | NSFDLDEQDF | GTCTTCCCAAAGAGGCATTGCCATCATTC |
| | | | ADIAKLDINLR | CAAAGTCCAACACTGTCCCAAGATTGTTG |
| | | | FNDPWDWDKI | GAAAACAAGGACGTCAACAGCTTCGACT |
| | | | PIFV (SEQ ID | TGGACGAACAAGATTTCGCTGACATTGCC |
| | | | NO: 48) | AAGTTGGACATCAACTTGAGATTCAACG |
| | | | | ACCCATGGGACTGGGACAAGATTCCTAT |
| | | | | CTTCGTCTAA (SEQ ID NO: 49) |
| XXDH | Pichia | XM_ | MTANPSLVLN | ATGACTGCTAACCCTTCCTTGGTGTTGAA |
| | stipitis | 001386945.1 | KIDDISEETYD | CAAGATCGACGACATTTCGTTCGAAACTT |
| | | | APEISEPTDVL | ACGATGCCCCAGAAATCTCTGAACCTACC |
| | | | VQVKKTGICG | GATGTCCTCGTCCAGGTCAAGAAAACCG |
| | | | SDIHFYAHGRI | GTATCTGTGGTTCCGACATCCACTTCTAC |
| | | | GNFVLTKPMV | GCCCATGGTAGAATCGGTAACTTCGTTTT |
| | | | LGHESAGTVV | GACCAAGCCAATGGTCTTGGGTCACGAA |
| | | | QVGKGVTSLK | TCCGCCGGTACTGTTGTCCAGGTTGGTAA |
| | | | VGDNVAIEPGI | GGGTGTCACCTCTCTTAAGGTTGGTGACA |
| | | | PSRFSDEYKSG | ACGTCGCTATCGAACCAGGTATTCCATCC |
| | | | HYNLCPHMAF | AGATTCTCCGACGAATACAAGAGCGGTC |
| | | | AATPNSKEGEP | ACTACAACTTGTGTCCTCACATGGCCTTC |
| | | | NPPGTLCKYF | GCCGCTACTCCTAACTCCAAGGAAGGCG |
| | | | KSPEDFLVKLP | AACCAAACCCACCAGGTACCTTATGTAA |
| | | | DHVSLELGAL | GTACTTCAAGTCGCCAGAAGACTTCTTGG |
| | | | VEPLSVGVHA | TCAAGTTGCCAGACCACGTCAGCTTGGA |
| | | | SKLGSVAFGD | ACTCGGTGCTCTTGTTGAGCCATTGTCTG |
| | | | YVAVFGAGPV | TTGGTGTCCACGCCTCTAAGTTGGGTTCC |
| | | | GLLAAAVAKT | GTTGCTTTCGGCGACTACGTTGCCGTCTT |
| | | | FGAKGVIVVDI | TGGTGCTGGTCCTGTTGGTCTTTTGGCTG |
| | | | FDNKLKMAK | CTGCTGTCGCCAAGACCTTCGGTGCTAAG |
| | | | DIGAATHTFNS | GGTGTCATCGTCGTTGACATTTTCGACAA |
| | | | KTGGSEELIKA | CAAGTTGAAGATGGCCAAGGACATTGGT |
| | | | FGGNVPNVVL | GCTGCTACTCACACCTTCAACTCCAAGAC |
| | | | ECTGAEPCIKL | CGGTGGTTCTGAAGAATTGATCAAGGCTT |
| | | | GVDAIAPGGR | TCGGTGGTAACGTGCCAAACGTCGTTTTG |
| | | | FVQVGNAAGP | GAATGTACTGGTGCTGAACCTTGTATCAA |
| | | | VSFPITVFAMK | GTTGGGTGTTGACGCCATTGCCCCAGGTG |
| | | | ELTLFGSFRYG | GTCGTTTCGTTCAAGTCGGTAACGCTGCT |
| | | | FNDYKTAVGIF | GGTCCAGTCAGCTTCCCAATCACCGTTTT |
| | | | DTNYQNGREN | CGCCATGAAGGAATTGACTTTGTTCGGTT |
| | | | APIDFEQLITHR | CTTTCAGATACGGATTCAACGACTACAAG |
| | | | YKFKDAIEAY | ACTGCTGTTGGAATCTTTGACACTAACTA |
| | | | DLVRAGKGAV | CCAAAACGGTAGAGAAAATGCTCCAATT |
| | | | KCLIDGPE(SEQ | GACTTTGAACAATTGATCACCCACAGATA |
| | | | ID NO: 50) | CAAGTTCAAGGACGCTATTGAAGCCTAC |
| | | | | GACTTGGTCAGAGCCGGTAAGGGTGCTG |
| | | | | TCAAGTGTCTCATTGACGGCCCTGAGTAA |
| | | | | (SEQ ID NO: 51) |
| XXK | Pichia | XM_ | MTTTPFDAPD | ATGACCACTACCCCATTTGATGCTCCAGA |
| | stipitis | 001387288.1 | KLFLGFDLSTQ | TAAGCTCTTCCTCGGGTTCGATCTTTCGA |
| | | | QLKIIVTDENL | CTCAGCAGTTGAAGATCATCGTCACCGAT |
| | | | AALKTYNVEF | GAAAACCTCGCTGCTCTCAAAACCTACA |
| | | | DSINSSVQKGV | ATGTCGAGTTCGATAGCATCAACAGCTCT |
| | | | IAINDEISKGAII | GTCCAGAAGGGTGTCATTGCTATCAACG |
| | | | SPVYMWLDAL | ACGAAATCAGCAAGGGTGCCATTATTTCC |
| | | | DHVFEDMKK | CCCGTTTACATGTGGTTGGATGCCCTTGA |
| | | | DGFPFNKVVGI | CCATGTTTTTGAAGACATGAAGAAGGAC |
| | | | SGSCQQHGSV | GGATTCCCCTTCAACAAGGTTGTTGGTAT |
| | | | YWSRTAEKVL | TTCCGGTTCTTGTCAACAGCACGGTTCGG |
| | | | SELDAESSLSS | TATACTGGTCTAGAACGGCCGAGAAGGT |
| | | | QMRSAFTFKH | CTTGTCCGAATTGGACGCTGAATCTTCGT |
| | | | APNWQDHSTG | TATCGAGCCAGATGAGATCTGCTTTCACC |
| | | | KELEEFERVIG | TTCAAGCACGCTCCAAACTGGCAGGATC |
| | | | ADALADISGSR | ACTCTACCGGTAAAGAGCTTGAAGAGTT |
| | | | AHYRFTGLQIR | CGAAAGAGTGATTGGTGCTGATGCCTTG |
| | | | KLSTRFKPEKY | GCTGATATCTCTGGTTCCAGAGCCCATTA |
| | | | NRTARISLVSS | CAGATTCACAGGGCTCCAGATTAGAAAG |
| | | | FVASVLLGRIT | TTGTCTACCAGATTCAAGCCCGAAAAGTA |
| | | | SIEEADACGM | CAACAGAACTGCTCGTATCTCTTTAGTTT |
| | | | NLYDIEKREFN | CGTCATTTGTTGCCAGTGTGTTGCTTGGT |
| | | | EELLAIAAGVH | AGAATCACCTCCATTGAAGAGGCCGATG |
| | | | PELDGVEQDG | CTTGTGGAATGAACTTGTACGATATCGAA |
| | | | EIYRAGINELK | AAGCGCGAGTTCAACGAAGAGCTCTTGG |
| | | | RKLGPVKPITY | CCATCGCTGCTGGTGTCCACCCTGAGTTG |
| | | | ESEGDIASYFV | GATGGTGTAGAACAAGACGGTGAAATTT |
| | | | TRYGFNPDCKI | ACAGAGCTGGTATCAATGAGTTGAAGAG |
| | | | YSFTGDNLATI | AAAGTTGGGTCCTGTCAAACCTATAACAT |
| | | | ISLPLAPNDALI | ACGAAAGCGAAGGTGACATTGCCTCTTA |
| | | | SLGTSTTVLIIT | CTTTGTCACCAGATACGGCTTCAACCCCG |
| | | | KNYAPSSQYH | ACTGTAAAATCTACTCGTTCACCGGAGAC |
| | | | LFKHPTMPDH | AATTTGGCCACGATTATCTCGTTGCCTTT |
| | | | YMGMICYCNG | GGCTCCAAATGATGCTTTGATCTCATTGG |
| | | | SLAREKVRDE | GTACTTCTACTACAGTTTTAATTATCACC |
| | | | VNEKFNVEDK | AAGAACTACGCTCCTTCTTCTCAATACCA |
| | | | KSWDKFNEIL | TTTGTTTAAACATCCAACCATGCCTGACC |
| | | | DKSTDFNNKL | ACTACATGGGCATGATCTGCTACTGTAAC |
| | | | GIYFPLGEIVPN | GGTTCCTTGGCCAGAGAAAAGGTTAGAG |
| | | | AAAQIKRSVL | ACGAAGTCAACGAAAAGTTCAATGTAGA |
| | | | NSKNEIVDVEL | AGACAAGAAGTCGTGGGACAAGTTCAAT |
| | | | GDKNWQPED | GAAATCTTGGACAAATCCACAGACTTCA |
| | | | DVSSIVESQTL | ACAACAAGTTGGGTATTTACTTCCCACTT |
| | | | SCRLRTGPMLS | GGCGAAATTGTCCCTAATGCCGCTGCTCA |
| | | | KSGDSSASSSA | GATCAAGAGATCGGTGTTGAACAGCAAG |
| | | | SPQPEGDGTDL | AACGAAATTGTAGACGTTGAGTTGGGCG |
| | | | HKVYQDLVK | ACAAGAACTGGCAACCTGAAGATGATGT |
| | | | KFGDLYTDGK | TTCTTCAATTGTAGAATCACAGACTTTGT |
| | | | KQTFESLTARP | CTTGTAGATTGAGAACTGGTCCAATGTTG |
| | | | NRCYYVGGAS | AGCAAGAGTGGAGATTCTTCTGCTTCCAG |
| | | | NNGSIIRKMGS | CTCTGCCTCACCTCAACCAGAAGGTGATG |
| | | | ILAPVNGNYK | GTACAGATTTGCACAAGGTCTACCAAGA |
| | | | VDIPNACALG | CTTGGTTAAAAAGTTTGGTGACTTGTACA |
| | | | GAYKASWSYE | CTGATGGAAAGAAGCAAACCTTTGAGTC |
| | | | CEAKKEWIGY | TTTGACCGCCAGACCTAACCGTTGTTACT |
| | | | DQYINRLFEVS | ACGTCGGTGGTGCTTCCAACAACGGCAG |
| | | | DEMNLFEVKD | CATTATCCGCAAGATGGGTTCCATCTTGG |
| | | | KWLEYANGV | CTCCCGTCAACGGAAACTACAAGGTTGA |
| | | | GMLAKMESEL | CATTCCTAACGCCTGTGCATTGGGTGGTG |
| | | | KH (SEQ ID NO: | CTTACAAGGCCAGTTGGAGTTACGAGTGT |
| | | | 52) | GAAGCCAAGAAGGAATGGATCGGATACG |
| | | | | ATCAGTATATCAACAGATTGTTTGAAGTA |
| | | | | AGTGACGAGATGAATCTGTTCGAAGTCA |
| | | | | AGGATAAATGGCTCGAATATGCCAACGG |
| | | | | GGTTGGAATGTTGGCCAAGATGGAAAGT |
| | | | | GAATTGAAACACTAA (SEQ ID NO: 53) |

Enhancement of the Pentose Phosphate Pathway

The pentose phosphate pathway (PP pathway) refers to a series of pathways for producing NADPH and pentose from a carbon source, and intermediate products of this pathway, such as sedoheptulose 7-phosphate (S7P), can be converted to mycosporine-like amino acids via the mycosporine-like amino acid biosynthesis pathway. Therefore, enhancing the pentose phosphate pathway to increase the production of cedheptrose 7-phosphate can increase the synthesis of mycosporine-like amino acids.

As used herein, enhancement of the pentose phosphate pathway means all actions that increases the amount of sedoheptulose 7-phosphate produced by the pentose phosphate pathway, and may be an effect associated with (i) increase in the production of sedoheptulose 7-phosphate and/or (ii) inhibition of conversion of sedoheptulose 7-phosphate to another substance (see FIG. 1a).

More specifically, the enhancement of the pentose phosphate pathway can be achieved through at least one selected from: an increase in the transketolase activity to produce sedoheptulose 7-phosphate in the pentose phosphate pathway, an increase in the protein activity that regulates NADPH production, and a reduction in the transaldolase activity involved in the conversion reaction between sedoheptulose 7-phosphate and glyceraldehyde 3-phosphate.

(i) Increase in the Production of Sedoheptulose 7-Phosphate

In one embodiment, the increase in the production of sedoheptulose 7-phosphate may be due to an increase in the activity of an enzyme involved in the production of sedoheptulose 7-phosphate in the pentose phosphate pathway. The “enhancement of activity” refers to the enhancement of the enzyme activity compared to its endogenous activity or its activity prior to modification (overexpression or introduction) of the microorganism (e.g., unmutated microorganism, or parent strain) due to overexpression of the gene encoding the enzyme and/or introduction of the enzyme or the gene encoding the same from the outside (homologous or heterologous).

In one embodiment, the enzyme or protein involved in the production of sedoheptulose 7-phosphate may be a transketase (e.g., TKL1) involved in the production of sedoheptulose 7-phosphate from a substrate (ribose-5-phosphate (R5P) or xylulose-5-phosphate (X5P) in the pentose phosphate pathway, a protein (e.g., Stb5), that regulates NADPH production and regulates oxidative stress, or a combination thereof, but is not limited thereto.

In one embodiment, the increase in the production of sedoheptulose 7-phosphate may be due to enhancing the activity of the transketolase (e.g., TKL1), Stb5, or both, but is not limited thereto.

In one specific embodiment, the enhancement of the activity of the enzyme or protein can be applied by various methods well known in the art. The method is not limited thereto, but may be one using genetic engineering or protein engineering.

The method for enhancing enzyme activity using the genetic engineering can be performed, for example, by the following methods:

1) a method for increasing the copy number in cells of the gene encoding the enzyme,

2) a method for modifying the expression regulatory sequence of the gene encoding the enzyme,

3) a method for modifying the base sequence of the initiation codon or 5′-UTR region of the enzyme,

4) a method for modifying a polynucleotide sequence on a chromosome such that the enzyme activity is enhanced,

5) a method for introducing a foreign polynucleotide exhibiting the activity of the enzyme or a codon-optimized mutant polynucleotide of the polynucleotide, or

6) a combination of the above methods, and the like, but are not limited thereto.

The method for enhancing the enzyme activity using the protein engineering can be performed, for example, by analyzing the tertiary structure of the protein to select an exposed site, and modifying or chemically modifying the exposed site, but is not limited thereto. The method (1) of increasing the copy number in cells of the gene encoding the enzyme can be performed by i) introducing a foreign gene (homologous and/or heterologous to the microorganism (host cell) into the host cell in a form operably linked to a vector, or ii) by inserting the foreign gene into a chromosome (genome) of the host cell, but the method is not particularly limited thereto. ii) When inserted into the chromosome (genome) of the host cell, the insertion position may be a position that does not affect the growth of the host cell (e.g., a non-transcriptional spacer (NTS), etc.) and/or a position that can increase insertion efficiency (e.g., retrotransposon, etc.), but the position is not limited thereto.

The foreign gene can be used without limitation in its origin or nucleic acid sequence as long as it exhibits an activity equivalent to that of an enzyme in a host cell. In one embodiment, the foreign gene may comprise a nucleic acid sequence that is codon-optimized to fit the host cell. The introduced gene may be 1 copy or more, for example, 1 to 20 copies, 1 to 15 copies, 1 to 10 copies, or 1 to 5 copies (e.g., 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, or 20 copies). When two or more genes are introduced, the introduced copy number of each gene may be the same as or different from each other. An enzyme is produced by expression of the introduced foreign gene in a host cell, so that in addition to the enzyme activity inherent in the host cell, its activity can be increased.

In the method 2) above, the modification of an expression regulatory sequence of the gene encoding the enzyme may be performed by inducing a modification in the sequence through deletion, insertion, or non-conservative or conservative substitution of a nucleic acid sequence, or through a combination thereof in order to further enhance the activity of the expression regulatory sequence, or by replacement with a nucleic acid sequence having a stronger activity, but is not particularly limited thereto. The expression regulatory sequence may comprise at least one selected frim the group consisting of a promoter, an operator sequence, a sequence coding for a ribosome-binding site, a sequence regulating the termination of transcription and translation, etc., but is not particularly limited thereto. When the gene is endogenous to a host cell, it may comprise an expression regulatory sequence that replaces or in addition to the original expression regulatory sequence (e.g., a promoter) and is modified so as to increase the expression of the gene. When the gene is a foreign gene (e.g., a foreign gene introduced into a host cell of method 1 above), the gene may be introduced into a form (such as a vector or expression cassette) that is operably linked to an expression regulatory sequence modified so as to increase the expression of the former.

In one embodiment, to increase gene expression, a strong homologous or heterologous promoter can be ligated upstream of the gene expression unit instead of the original promoter. Examples of the strong promoter comprise TDH3 promoter (Saccharomyces cerevisiae), TEF1 (translation elongation factor 1, Saccharomyces cerevisiae) ADH1 promoter (Saccharomyces cerevisiae), CJ7 promoter, lysCP1 promoter, EF-Tu promoter, groEL promoter, aceA or aceB promoter, and the like. For example, the promoter may be a promoter derived frim the genus Corynebacterium, lysCP1 promoter (WO2009/096689), CJ7 promoter (WO2006/065095), SPL promoter (KR 10-1783170 B), or o2 promoter (KR 10-1632642 B), but are not limited thereto.

(ii) Inhibition of Conversion of Sedoheptulose 7-Phosphate to Other Substances

In another embodiment, the inhibition of the conversion of sedoheptulose 7-phosphate to other substances may be due to inhibition of the activity of enzymes involved in the reaction of converting (metabolizing or fermenting) sedoheptulose 7-phosphate to other products in the pentose phosphate pathway. The “activity inhibition” may mean that by inhibiting the expression of the gene encoding the enzyme and/or inactivating the enzyme activity, etc., the activity is reduced or the enzyme activity is lost as compared to the endogenous activity of the microorganism or the activity prior to modification (expression inhibition or introduction) (e.g., unmutated microorganism).

In one specific embodiment, the protein or enzyme involved in the reaction of converting the sedoheptulose 7-phosphate to another product may be a transaldolase (e.g., TAL1) involved in the conversion reaction between sedoheptulose 7-phosphate and glyceraldehyde 3-phosphate (G3P) in the pentose phosphate pathway, but is not limited thereto.

In one specific embodiment, the inhibition of conversion of sedoheptulose 7-phosphate to other substances may be due to one or more (e.g., one, two, three, four, five, or six) selected from the group consisting of:

1′) mutation (e.g., a deletion, one or more nucleic acid substitutions, and/or one or more nucleic acid insertions) of a gene encoding the enzyme,

2′) modification of the expression regulatory sequence so as to reduce the expression of the gene encoding the enzyme,

3′) modification of the gene sequence encoding the enzyme so that the activity of the enzyme is removed or weakened,

4′) introduction of an antisense oligonucleotide (e.g., antisense RNA) that complementarily binds to the transcript of the gene encoding the enzyme, and

5′) addition of a sequence complementary to the Shine-Dalgamo sequence to the front end of the Shine-Dalgamo sequence of the gene encoding the enzyme to form a secondary structure, thereby inhibiting or preventing the attachment of ribosomes, and

6′) addition of a promoter transcribed in the opposite direction to the 3′end of ORF (open reading frame) of the gene encoding the enzyme (reverse transcription engineering, RT), and the like, but are not limited thereto. In one embodiment, the genes encoding the enzymes of the above 1) to 6) may be genes inherent in the microbial genome (chromosome), without being limited thereto.

The 1′) deletion of the gene encoding the enzyme (transaldolase; for example, TAL1) means a complete deletion of the gene or a partial deletion of the gene that prevents expression of the enzyme (transaldolase), or a partial deletion of the gene that prevents the expressed enzyme from having its original full function. In one specific embodiment, the partial deletion may mean that one or more 5 or more, 10 or more, 15 more, 20 or more, 25 or more, 30 or more, 35 or more, 40 more, 45 or more, or 50 or more, for example, 1 to 500, 5 to 500, 10 to 500, 15 to 500, 20 to 500, 25 to 500, 30 to 500, 35 to 500, 40 to 500, 45 to 500, 50 to 500, 1 to 200, 5 to 200, 10 to 200, 15 to 200, 20 to 200, 25 to 200, 30 to 200, 35 to 200, 40 to 200, 45 to 200, 50 to 200, 1 to 100, 5 to 100, 10 to 100, 15 to 100, 20 to 100, 25 to 100, 30 to 100, 35 to 100, 40 to 100, 45 to 100, 50 to 100, 1 to 50, 5 to 50, 10 to 50, 15 to 50, 20 to 50, 25 to 50, 30 to 50, 35 to 50, 40 to 50, or 45 to 50 nucleic acids (nucleotides), which is consecutive or non-contiguous in a gene, can be deleted, but is not limited thereto.

The 2′) modification of the expression regulatory sequence so as to reduce the expression of the gene encoding the enzyme, or the 3′) modification of the gene sequence encoding the enzyme so that the activity of the enzyme is removed or weakened can be performed by mutating the nucleic acid sequence by deletion, insertion, non-conservative or conservative substitution of all or part of the nucleic acid, or a combination thereof, or by replacing the nucleic acid sequence with an expression regulatory sequence or gene having weaker activity, such that the activity of the expression regulatory sequence is weakened or the coding gene encodes an enzyme whose activity has been removed or weakened. The expression regulatory sequence comprises a promoter, an operator sequence, a sequence encoding a ribosome binding site, and a sequence regulating the termination of transcription and translation, but is not limited thereto.

In one embodiment, the mutant microorganism having enhanced pentose phosphate pathway may be a microorganism which comprises:

overexpression of a gene encoding a transketolase (e.g., TKL1), a gene encoding Stb5, or both;

deletion of all or part of the gene encoding a transaldolase (e.g., TAL1); or

a combination thereof.

In one specific embodiment, the transketolase (e.g., TKL1), Stb5, and transaldolase (e.g., TAL1) may be derived from Saccharomyces cerevisiae, but is not limited thereto.

Enzymes or proteins usable for enhancing the pentose phosphate pathway, and genes encoding the same are exemplified in Table 2 below:

TABLE 2

| | | GenBank | | |
|---|---|---|---|---|
| | | Accession | Amino acid | |
| Enzyme | Origin | no. | sequence (N→C) | Nucleic acid sequence(5′→3′) |
| TKL1 | Saccharo- | JRIV0100 | MTQFTDIDKLA | ATGACTCAATTCACTGACATTGATAAGCTA |
| | myces | 0030.1 | VSTIRILAVDTVS | GCCGTCTCCACCATAAGAATTTTGGCTGTG |
| | cerevisiae | | KANSGHPGAPL | GACACCGTATCCAAGGCCAACTCAGGTCA |
| | | | GMAPAAHVLW | CCCAGGTGCTCCATTGGGTATGGCACCAGC |
| | | | SQMRMNPTNPD | TGCACACGTTCTATGGAGTCAAATGCGCAT |
| | | | WINRDRFVLSN | GAACCCAACCAACCCAGACTGGATCAACA |
| | | | GHAVALLYSML | GAGATAGATTTGTCTTGTCTAACGGTCACG |
| | | | HLTGYDLSIEDL | CGGTCGCTTTGTTGTATTCTATGCTACATTT |
| | | | KQFRQLGSRTPG | GACTGGTTACGATCTGTCTATTGAAGACTT |
| | | | HPEFELPGVEVT | GAAACAGTTCAGACAGTTGGGTTCCAGAA |
| | | | TGPLGQGISNAV | CACCAGGTCATCCTGAATTTGAGTTGCCAG |
| | | | GMAMAQANLA | GTGTTGAAGTTACTACCGGTCCATTAGGTC |
| | | | ATYNKPGFTLSD | AAGGTATCTCCAACGCTGTTGGTATGGCCA |
| | | | NYTYVFLGDGC | TGGCTCAAGCTAACCTGGCTGCCACTTACA |
| | | | LQEGISSEASSLA | ACAAGCCGGGCTTTACCTTGTCTGACAACT |
| | | | GHLKLGNLIAIY | ACACCTATGTTTTCTTGGGTGACGGTTGTTT |
| | | | DDNKITIDGATSI | GCAAGAAGGTATTTCTTCAGAAGCTTCCTC |
| | | | SFDEDVAKRYE | CTTGGCTGGTCATTTGAAATTGGGTAACTT |
| | | | AYGWEVLYVE | GATTGCCATCTACGATGACAACAAGATCAC |
| | | | NGNEDLAGIAK | TATCGATGGTGCTACCAGTATCTCATTCGA |
| | | | AIAQAKLSKDKP | TGAAGATGTTGCTAAGAGATACGAAGCCT |
| | | | TLIKMTTTIGYG | ACGGTTGGGAAGTTTTGTACGTAGAAAATG |
| | | | SLHAGSHSVHG | GTAACGAAGATCTAGCCGGTATTGCCAAG |
| | | | APLKADDVKQL | GCTATTGCTCAAGCTAAGTTATCCAAGGAC |
| | | | KSKFGFNPDKSF | AAACCAACTTTGATCAAAATGACCACAAC |
| | | | VVPQEVYDHYQ | CATTGGTTACGGTTCCTTGCATGCCGGCTC |
| | | | KTILKPGVEANN | TCACTCTGTGCACGGTGCCCCATTGAAAGC |
| | | | KWNKLFSEYQK | AGATGATGTTAAACAACTAAAGAGCAAAT |
| | | | KEPELGAELARR | TCGGTTTCAACCCAGACAAGTCCTTTGTTG |
| | | | LSGQLPANWES | TTCCACAAGAAGTTTACGACCACTACCAAA |
| | | | KLPTYTAKDSA | AGACAATTTTAAAGCCAGGTGTCGAAGCC |
| | | | VATRKLSETVLE | AACAACAAGTGGAACAAGTTGTTCAGCGA |
| | | | DVYNQLPELIGG | ATACCAAAAGAAATTCCCAGAATTAGGTG |
| | | | SADLTPSNLTR | CTGAATTGGCTAGAAGATTGAGCGGCCAA |
| | | | WKEALDFQPPSS | CTACCCGCAAATTGGGAATCTAAGTTGCCA |
| | | | GSGNYSGRYIRY | ACTTACACCGCCAAGGACTCTGCCGTGGCC |
| | | | GIREHAMGAIM | ACTAGAAAATTATCAGAAACTGTTCTTGAG |
| | | | NGISAFGANYKP | GATGTTTACAATCAATTGCCAGAGTTGATT |
| | | | YGGTFLNFVSY | GGTGGTTCTGCCGATTTAACACCTTCTAAC |
| | | | AAGAVRLSALS | TTGACCAGATGGAAGGAAGCCCTTGACTTC |
| | | | GHPVIWVATHD | CAACCTCCTTCTTCCGGTTCAGGTAACTAC |
| | | | SIGVGEDGPTHQ | TCTGGTAGATACATTAGGTACGGTATTAGA |
| | | | PIETLAHFRSLPN | GAACACGCTATGGGTGCCATAATGAACGG |
| | | | IQVWRPADGNE | TATTTCAGCTTTCGGTGCCAACTACAAACC |
| | | | VSAAYKNSLES | ATACGGTGGTACTTTCTTGAACTTCGTTTCT |
| | | | KHTPSIIALSRQN | TATGCTGCTGGTGCCGTTAGATTGTCCGCT |
| | | | LPQLEGSSIESAS | TTGTCTGGCCACCCAGTTATTTGGGTTGCT |
| | | | KGGYVLQDVA | ACACATGACTCTATCGGTGTCGGTGAAGAT |
| | | | NPDIILVATGSE | GGTCCAACACATCAACCTATTGAAACTTTA |
| | | | VSLSVEAAKTL | GCACACTTCAGATCCCTACCAAACATTCAA |
| | | | AAKNIKARVVS | GTTTGGAGACCAGCTGATGGTAACGAAGTT |
| | | | LPDHIFDKQPL | TCTGCCGCCTACAAGAACTCTTTAGAATCC |
| | | | EYRLSVLPDNVP | AAGCATACTCCAAGTATCATTGCTTTGTCC |
| | | | IMSVEVLATTC | AGACAAAACTTGCCACAATTGGAAGGTAG |
| | | | WGKYAHQSFGI | CTCTATTGAAAGCGCTTCTAAGGGTGGTTA |
| | | | DRFGASGKAPE | CGTACTACAAGATGTTGCTAACCCAGATAT |
| | | | VFKFFGFTPEGV | TATTTTAGTGGCTACTGGTTCCGAAGTGTC |
| | | | AERAQKTIAFYK | TTTGAGTGTTGAAGCTGCTAAGACTTTGGC |
| | | | GDKLISPLKKAF | CGCAAAGAACATCAAGGCTCGTGTTGTTTC |
| | | | (SEQ ID NO: 54) | TCTACCAGATTTCTTCACTTTTGACAAACA |
| | | | | ACCCCTAGAATACAGACTATCAGTCTTACC |
| | | | | AGACAACGTTCCAATCATGTCTGTTGAAGT |
| | | | | TTTGGCTACCACATGTTGGGGCAAATACGC |
| | | | | TCATCAATCCTTCGGTATTGACAGATTTGG |
| | | | | TGCCTCCGGTAAGGCACCAGAAGTCTTCAA |
| | | | | GTTCTTCGGTTTCACCCCAGAAGGTGTTGC |
| | | | | TGAAAGAGCTCAAAAGACCATTGCATTCTA |
| | | | | TAAGGGTGACAAGCTAATTTCTCCTTTGAA |
| | | | | AAAAGCTTTCTAA (SEQ ID NO: 55) |
| Stb5 | | JRIV0100 | MDGPNFAHQGG | ATGGATGGTCCCAATTTTGCACATCAAGGC |
| | | 0036.1 | RSQRTTELYSCA | GGGAGATCACAACGTACTACTGAATTGTAT |
| | | | RCRKLKKKCGK | TCGTGCGCACGATGCAGAAAATTAAAGAA |
| | | | QIPTCANCDKN | GAAGTGTGGTAAACAAATACCGACATGTG |
| | | | GAHCSYPGRAP | CAAACTGCGATAAAAATGGGGCACACTGT |
| | | | RRTKKELADAM | TCATATCCAGGTAGAGCCCCAAGACGTACC |
| | | | LRGEYVPVKRN | AAGAAGGAGTTAGCGGATGCTATGCTACG |
| | | | KKVGKSPLSTKS | AGGGGAATATGTTCCAGTGAAAAGGAACA |
| | | | MPNSSSPLSANG | AGAAGGTAGGAAAAAGCCCATTGAGCACT |
| | | | AITPGFSPYEND | AAGAGCATGCCAAACTCTTCTAGTCCGCTA |
| | | | DAHKMKQLKPS | TCCGCAAATGGCGCTATAACTCCCGGGTTT |
| | | | DPINLVMGASPN | TCGCCTTACGAAAACGATGATGCACATAA |
| | | | SSEGVSSLISVLT | GATGAAACAGCTAAAACCGTCAGATCCAA |
| | | | SLNDNSNPSSHL | TAAATCTTGTCATGGGGGCAAGTCCAAATT |
| | | | SSNENSMIPSRSL | CTAGCGAAGGTGTCTCATCGCTAATTTCGG |
| | | | PASVQQSSTTSS | TGCTAACATCGCTGAATGATAATTCTAATC |
| | | | FGGYNTPSPLISS | CTTCTTCGCACTTATCCTCTAATGAAAATTC |
| | | | HVPANAQAVPL | CATGATTCCTTCCCGATCATTGCCAGCTTCC |
| | | | QNNNRNTSNGD | GTGCAGCAAAGTTCGACAACTTCATCATTC |
| | | | NGSNVNHDNNN | GGAGGATATAACACGCCTTCACCACTAATT |
| | | | GSTNTPQLSLTP | AGCAGTCATGTGCCTGCGAACGCCCAAGC |
| | | | YANNSAPNGKF | CGTACCGCTACAAAACAACAATCGCAATA |
| | | | DSVPVDASSIEF | CTAGCAACGGGGATAACGGCAGTAATGTT |
| | | | ETMSCCFKGGR | AACCACGATAATAACAATGGCAGTACCAA |
| | | | TTSWVREDGSF | CACACCGCAATTGAGTCTTACCCCATATGC |
| | | | KSIDRSLLDRFIA | AAACAATTCAGCCCCCAATGGGAAATTCG |
| | | | AYFKHNHRLFP | ATTCTGTGCCGGTTGATGCATCCTCGATCG |
| | | | MIDKIAFLNDAA | AATTTGAAACTATGTCCTGTTGCTTTAAAG |
| | | | TITDFERLYDNK | GTGGTAGAACAACATCGTGGGTCAGAGAG |
| | | | NYPDSFVFKVY | GATGGCTCGTTCAAGTCAATTGATAGATCC |
| | | | MIMAIGCTTLQR | TTACTGGACAGGTTCATTGCCGCATACTTC |
| | | | AGMVSQDEECL | AAACACAATCACCGTCTATTTCCCATGATT |
| | | | SEHLAFLAMKK | GATAAAATAGCATTCCTAAATGACGCCGC |
| | | | FRSVIILQDIETV | GACAATTACTGATTTCGAAAGGTTATATGA |
| | | | RCLLLLGIYSFFE | CAACAAAAACTACCCTGACAGCTTTGTTTT |
| | | | PKGSSSWTISGII | CAAAGTATACATGATCATGGCTATTGGTTG |
| | | | MRLTIGLGLNRE | TACAACTTTACAGCGTGCTGGTATGGTTTC |
| | | | LTAKKLKSMSA | TCAGGACGAAGAGTGTCTGAGTGAACATTT |
| | | | LEAEARYRVFW | GGCTTTTTTGGCCATGAAAAAATTTCGTAG |
| | | | SAYCFERLVCTS | TGTTATAATTTTACAAGATATCGAAACTGT |
| | | | LGRISGIDDEDIT | ACGATGCCTATTGTTGTTGGGTATTTATTCG |
| | | | VPLPRALYVDE | TTTTTTGAGCCAAAGGGCTCCTCGTCATGG |
| | | | RDDLEMTKLMI | ACAATTAGTGGTATCATCATGCGATTGACC |
| | | | SLRKMGGRIYK | ATAGGATTAGGTTTAAATAGAGAGTTAACT |
| | | | QVHSVSAGRQK | GCCAAAAAACTCAAGAGCATGTCTGCTTTA |
| | | | LTIEQKQEIISGL | GAAGCAGAGGCAAGATATAGAGTGTTTTG |
| | | | RKELDEIYSRES | GAGTGCTTACTGCTTTGAAAGGCTAGTATG |
| | | | ERRKLKKSQMD | CACCTCGTTGGGCCGTATATCCGGGATCGA |
| | | | QVERENNSTTN | CGACGAAGACATCACTGTGCCACTACCGA |
| | | | VISFHSSEIWLA | GGGCGTTGTATGTGGATGAAAGAGACGAT |
| | | | MRYSQLQILLYR | TTAGAGATGACCAAGTTGATGATATCATTA |
| | | | PSALMPKPPIDS | AGGAAAATGGGCGGTCGCATTTATAAACA |
| | | | LSTLGEFCLQA | AGTCCACTCTGTAAGTGCAGGGCGACAAA |
| | | | WKHTYTLYKKR | AGTTAACCATCGAACAAAAGCAGGAAATC |
| | | | LLPLNWITLFRT | ATAAGTGGATTACGCAAGGAGCTAGACGA |
| | | | LTICNTILYCLCQ | AATTTATTCTCGAGAATCAGAAAGAAGGA |
| | | | WSIDLIESKIEIQ | AACTGAAAAAATCTCAAATGGATCAGGTG |
| | | | QCVEILRHFGER | GAGAGGGAGAACAATTCTACTACAAATGT |
| | | | WIFAMRCADVF | AATATCCTTCCATAGTTCTGAGATTTGGCT |
| | | | QNISNTILDISLS | AGCAATGAGATACTCCCAGTTGCAAATCTT |
| | | | HGKVPNMDQLT | ACTATACAGACCATCTGCATTGATGCCAAA |
| | | | RELFGASDSYQ | ACCGCCCATTGACTCACTATCCACTCTAGG |
| | | | DILDENNVDVS | AGAGTTTTGCTTGCAAGCCTGGAAACATAC |
| | | | WVDKLV (SEQ | TTATACACTGTACAAGAAGCGGTTATTACC |
| | | | ID NO: 56) | CTTGAACTGGATAACCCTTTTCAGAACATT |
| | | | | AACCATTTGTAACACTATCTTATACTGTCTT |
| | | | | TGCCAGTGGAGCATCGACCTCATTGAAAGT |
| | | | | AAAATCGAAATTCAACAGTGTGTGGAAAT |
| | | | | ACTAAGACATTTCGGTGAAAGATGGATTTT |
| | | | | TGCTATGAGATGCGCGGATGTTTTCCAAAA |
| | | | | CATTAGCAACACAATTCTCGATATTAGTTT |
| | | | | AAGCCATGGTAAAGTTCCTAATATGGACCA |
| | | | | ATTAACAAGAGAGTTATTTGGCGCCAGCG |
| | | | | ATTCATATCAAGACATATTAGACGAAAAC |
| | | | | AATGTTGACGTCTCTTGGGTTGATAAACTT |
| | | | | GTATGA (SEQ ID NO: 57) |
| TAL1 | | JRIV0100 | MSEPAQKKQKV | ATGTCTGAACCAGCTCAAAAGAAACAAAA |
| | | 0052.1 | ANNSLEQLKAS | GGTTGCTAACAACTCTCTAGAACAATTGAA |
| | | | GTVVVADTGDF | AGCCTCCGGCACTGTCGTTGTTGCCGACAC |
| | | | GSIAKFQPQDST | TGGTGATTTCGGCTCTATTGCCAAGTTTCA |
| | | | TNPSLILAAAKQ | ACCTCAAGACTCCACAACTAACCCATCATT |
| | | | PTYAKLIDVAVE | GATCTTGGCTGCTGCCAAGCAACCAACTTA |
| | | | YGKKHGKTTEE | CGCCAAGTTGATCGATGTTGCCGTGGAATA |
| | | | QVENAVDRLLV | CGGTAAGAAGCATGGTAAGACCACCGAAG |
| | | | EFGKEILKIVPGR | AACAAGTCGAAAATGCTGTGGACAGATTG |
| | | | VSTEVDARLSFD | TTGGTCGAATTCGGTAAGGAGATCTTAAAG |
| | | | TQATIEKARHIIK | ATTGTTCCAGGCAGAGTCTCCACCGAAGTT |
| | | | LFEQEGVSKERV | GATGCTAGATTGTCTTTTGACACTCAAGCT |
| | | | LIKIASTWEGIQ | ACCATTGAAAAGGCTAGACATATCATTAA |
| | | | AAKELEEKDGIH | ATTGTTTGAACAAGAAGGTGTCTCCAAGGA |
| | | | CNLTLLFSFVQA | AAGAGTCCTTATTAAAATTGCTTCCACTTG |
| | | | VACAEAQVTLIS | GGAAGGTATTCAAGCTGCCAAAGAATTGG |
| | | | PFVGRILDWYKS | AAGAAAAGGACGGTATCCACTGTAATTTG |
| | | | STGKDYKGEAD | ACTCTATTATTCTCCTTCGTTCAAGCAGTTG |
| | | | PGVISVKKIYNY | CCTGTGCCGAGGCCCAAGTTACTTTGATTT |
| | | | YKKYGYKTIVM | CCCCATTTGTTGGTAGAATTCTAGACTGGT |
| | | | GASFRSTDEIKN | ACAAATCCAGCACTGGTAAAGATTACAAG |
| | | | LAGVDYLTISPA | GGTGAAGCCGACCCAGGTGTTATTTCCGTC |
| | | | LLDKLMNSTEPF | AAGAAAATCTACAATTACTACAAGAAGTA |
| | | | PRVLDPVSAKK | CGGTTACAAGACTATTGTTATGGGTGCTTC |
| | | | EAGDKISYISDES | TTTCAGAAGCACTGACGAAATCAAAAACTT |
| | | | KFRFDLNEDAM | GGCTGGTGTTGACTATCTAACAATTTCTCC |
| | | | ATEKLSEGIRKF | AGCTTTATTGGACAAGTTGATGAACAGTAC |
| | | | SADIVTLFDLIEK | TGAACCTTTCCCAAGAGTTTTGGACCCTGT |
| | | | KVTA (SEQ ID | CTCCGCTAAGAAGGAAGCCGGCGACAAGA |
| | | | NO: 58) | TTTCTTACATCAGCGACGAATCTAAATTCA |
| | | | | GATTCGACTTGAATGAAGACGCTATGGCCA |
| | | | | CTGAAAAATTGTCCGAAGGTATCAGAAAA |
| | | | | TTCTCTGCCGATATTGTTACTCTATTCGACT |
| | | | | TGATTGAAAAGAAAGTTACCGCTTAA (SEQ |
| | | | | ID NO: 59) |

Mycosporine-Like Amino Acid

As used herein, “mycosporine-like amino acid (MAA)” refers to a cyclic compound that absorbs ultraviolet light. In the present disclosure, mycosporine-like amino acid is not limited as long as it can absorb ultraviolet light, but specifically, it may be a compound having a central ring of cyclohexanone or cyclohexanamine, or a compound in which various substances such as amino acids are bound to the central ring. Examples thereof may be mycosporine-2-glycine, palythinol, palythenic acid, deoxygadusol, mycosporine-methylaminethreonine, mycosporine-glycine-valine, palythine, asterina-330, shinorine, porphyra-334, euhalothece-362, mycosporine-glycine, mycosporine-ornithine, mycosporine-lysine, mycosporine-glutamic acid-glycine, mycosporine-methylamine-serine, mycosporine-taurine, palythene, palythine-serine, palythine-serine-sulfate, palythinol, usujirene or a combination thereof.

Microorganisms that Produce Mycosporine-Like Acids and Mycosporine-Like Amino Acid Biosynthesis Genes

As used herein, the “microorganism” may refer to a prokaryotic or eukaryotic microorganism (unicellular organism), and specifically, it may mean a microorganism that produces the mycosporine-like amino acid.

The “microorganism that produces mycosporine-like amino acids” may mean a microorganism comprising a mycosporine-like amino acid biosynthesis gene or a cluster of the gene. The microorganism may mean a microorganism in which the mycosporine-like amino acid biosynthesis gene or the cluster is introduced or enhanced.

As used herein, the “mycosporine-like amino acid biosynthesis gene” may mean a gene selected from among genes encoding enzymes involved in mycosporine-like amino acid biosynthesis pathway, and can comprise not only any one gene selected among the genes encoding enzymes involved in the biosynthesis of mycosporine-like amino acids, but also a mycosporine-like amino acid biosynthesis gene cluster containing two or more, e.g., two, three, four, or five. The mycosporine-like amino acid biosynthesis gene comprises both exogenous and/or endogenous genes of microorganisms as long as a microorganism containing the same can produce mycosporine-like amino acids. The foreign gene may be homologous and/or heterologous.

The mycosporine-like amino acid biosynthesis gene is not limited in the microbial species from which the above genes are derived, as long as the microorganism containing the same can produce an enzyme involved in the biosynthesis of mycosporine-like amino acid and consequently produce mycosporine-like amino acids. Examples thereof may be, as the cyanobacteria, Anabaena variabiis, Nostoc punctiforme, Nodularia spumigena, Cyanothece sp. PCC 7424, Lyngbya sp. PCC 8106, Microcystis aeruginosa, Microcoleus chthonoplastes, Cyanothece sp. ATCC 51142, Crocosphaera watsonii, Cyanothece sp. CCY 0110, Cylindrmspermum stagnale sp, PCC 7417, Aphanothece halophyica or Trichodesmium erythraeum; or as the fungi, Magnaporthe ozyae, Pyrenophom tritici-repentis, Aspergillus clavatus, Nectria haematococca, Aspergillus nidulans, Gibberella zeae, Verticilium albo-atrum, Botryotinia fuckeliana, Phaeosphaeria nodorum; or Nematostella vectensis, Heterocapsa triquetra, Oxyrrhis marina, Karlodinium micrum, or Actinosynnema mirum, or the like, but is not limited thereto.

According to one embodiment, the microorganism producing the mycosporine-like amino acid described herein may comprise a mycosporine-like amino acid biosynthesis enzyme, or a gene encoding the same or a cluster of the gene. For example, the microorganism may be one in which an exogenous (homologous or heterologous) mycosporine-like amino acid biosynthetic gene cluster was introduced, or one in which the activity of the protein encoded by the gene may be improved as compared to the endogenous activity or the activity prior to modification, but is not limited thereto.

In one embodiment, the mycosporine-like amino acid biosynthesis enzyme is not limited to the name of the enzyme or the derived microorganism, as long as the microorganism containing the same can produce mycosporine-like amino acids, and examples thereof may comprise one or more, for example, 1 or more, 2 or more, 3 or more, 4 or more, or all of them, selected from the group consisting of:

an enzyme that converts sedoheptulose 7-phosphate (S7P) to 2-dimethyl-4-deoxygadusol (DDG), for example, an enzyme that converts 2-dimethyl 4-deoxygadusol synthase (DDGS), 2-dimethyl-4-deoxygadusol to 4-deoxygadusol (4-DG), for example, O-methyltransferase (O-MT), and

an enzyme that catalyzes the glycylation (glycine bond) of 4-deoxygadusol to form mycosporine-glycine (MG), for example, ATP-grasp ligase, more specifically C—N ligase.

In addition, the microorganism producing the mycosporine-like amino acid may comprise genes of enzymes or clusters of said genes that have the activity of attaching additional amino acid residues to mycosporine-like amino acids.

The mycosporine-like amino acid biosynthesis enzyme is not limited to the name of the enzyme or the derived microorganism as long as microorganisms that produce mycosporine-like amino acids can produce mycosporine-like amino acids to which two or more amino acid residues are attached. For example, the enzyme may comprise one or more, specifically, one, two, or three, selected from the group consisting of non-ribosomal peptide synthetase (NRPS), non-ribosomal peptide synthetase-like enzyme (NRPS-like enzyme), and D-Ala D-Ala ligase (DDL).

Some mycosporine-like amino acids contain a second amino acid residue in mycosporine-glycine. The at least one enzyme selected from the group consisting of non-ribosomal peptide synthetase, non-ribosomal peptide synthetase-like enzyme, and D-Ala D-Ala ligase can attach a second amino acid residue to mycosporine-glycine.

According to one embodiment, the enzyme having the activity of attaching an additional amino aid residue to the mycosporine-like amino acid is not limited to the enzyme name or the derived microbial species if it is non-ribosomal peptide synthetase, non-ribosomal peptide synthetase-like enzyme, and D-Ala D-Ala ligase, and may comprise one or more, for example, one, two, or three, selected among these enzymes.

For example, Anabaena variabilis-derived non-ribosomal peptide synthetase-like enzyme (Ava_3855) or Nostoc punctiforme-derived D-Ala D-Ala ligase (NpF5597) can attach a serine residue to mycosporine-glycine to form shinorine. In another embodiment, mycosporine-2-glycine can be formed by an attachment of a second glycine residue by Aphanothece halophytica-derived D-Ala D-Ala ligase homolog (Ap_3855). Similarly, in Acdnosynnema mirum, serine or alanine can be attached to D-Ala D-Ala ligase to form shinorine or mycosporine-glycine-alanine.

The microorganism according to an embodiment of the present disclosure may select and comprise the aforementioned enzymes or enzymes having an activity identical and/or similar thereto, and/or those suitable for the production of the desired mycosporine-like amino acid among genes encoding the same.

In one embodiment, the mycosporine-like amino acid biosynthesis enzyme may comprise one or more, for example, 1, 2, 3, 4, or 5 genes, selected from the group consisting of:

2-dimethyl 4-deoxygadusol synthase (DDGS),

O-methyltransferase (O-MT),

C—N ligase and/or ATP-grasp ligase, and

non-ribosomal peptide synthetase, non-ribosomal peptide synthetase-like enzyme, and/or D-Ala D-Ala ligase.

In one embodiment, the gene may be derived from Nostoc punctiforme or Anabaena variabilis.

The 2-dimethyl 4-deoxygadusol synthetase, O-methyl transferase, C—N ligase, ATP-grasp ligase, non-ribosomal peptide synthetase, non-ribosomal peptide synthetase-like enzyme, and D-Ala D-Ala ligase are not limited to the microbial species from which they are derived, and are not limited as long as they are known as enzymes that perform the same and/or similar functions and roles, and the range of homology values between them is also not limited.

For example, MylA, MylB, MylD, MylE and MylC of Cylindrospermum stagnale sp, PCC 7417 (C. stagnale PCC 7417) are homologous to Anabaena variabilis and Nostoc punctiforme-derived 2-dimethyl 4-deoxygadusol synthase, O-methyltransferase, C—N ligase, and D-alanine D, and the similarity between them is about 61 to 88% (Appl Environ Microbiol, 2016, 82(20), 6167-6173; J Bacteriol, 2011, 193(21), 5923-5928). That is, the enzymes that can be used herein are not significantly limited to the derived microbial species or sequence homology as long as they are known to exhibit identical and/or similar functions and effects.

For example, Nostoc punctiforme ATCC 29133 and Anabaena variabilis ATCC 29413 use sedoheptulose 7-phosphate (S7P) as an intermediate, to produce a mycosporine-like amino acid (e.g., shinorine) (see FIG. 1). S7P, an intermediate of the pentose phosphate pathway, is converted to a mycosporine-like amino acid (e.g., shinorine) through an enzymatic reaction in four steps: First, S7P is converted to a 4-deoxygadusol (4-DG) by 2-dimethyl 4-deoxygadusol synthase (DDGS) and O-methyltransferase (O-MT). Next, glycine is bound to the 4-DG by ATP-grasp ligase to form mycosporine-glycine (MG). Finally, by enzymes such as D-ala-D-ala ligase of Nostoc punctiforme non-ribosomal peptide synthetase (NRPS) of Anabaena variabilis, 1 serine is attached to the formed MG to form shinorine.

The mycosporine-like amino acid biosynthesis enzymes and genes encoding the same are exemplified in Table 3 below:

TABLE 3

| | | GenBank | Aminoacid | |
|---|---|---|---|---|
| | | Accession | sequence | |
| Enzyme | Origin | no. | (N→C) | Nucleic acid sequence(5′→3′) |
| DDGS | Nostoc | ACC83905.1 | MSNVQASFEA | ATGAGTAATGTTCAAGCATCGTTTGAAGC |
| | punctiforme | | TEAEFRVEGY | AACGGAAGCTGAATTCCGCGTGGAAGGT |
| | or | | EKIEFSLVYVN | TACGAAAAAATTGAGTTTAGTCTTGTCTA |
| | Anabaena | | GAFDISNREIA | TGTAAATGGTGCATTTGATATCAGTAACA |
| | variabilis | | DSYEKFGRCL | GAGAAATTGCAGACAGCTATGAGAAGTT |
| | | | TVIDANVNRL | TGGCCGTTGTCTGACTGTGATTGATGCTA |
| | | | YGKQIKSYFR | ATGTCAACAGATTATATGGCAAGCAAAT |
| | | | HYGIDLTWPI | CAAGTCATATTTTAGACACTATGGTATTG |
| | | | VITEPTKTLAT | ATCTGACCGTAGTTCCCATTGTGATTACT |
| | | | FEKIVDAFSDF | GAGCCTACTAAAACCCTTGCAACCTTTGA |
| | | | GLIRKEPVLVV | GAAAATTGTTGATGCTTTTTCTGACTTTG |
| | | | GGGLTTDVAG | GTTTAATCCGCAAGGAACCAGTATTAGTA |
| | | | FACAAYRRKS | GTGGGTGGTGGTTTAACCACTGATGTAGC |
| | | | NYIRVPTTLIG | TGGGTTTGCGTGTGCTGCTTACCGTCGTA |
| | | | LIDAGVAIKVA | AGAGTAACTATATTCGGGTTCCGACAAC |
| | | | VNHRKLKNRL | GCTGATTGGTCTGATTGATGCAGGTGTAG |
| | | | GAYHAPLKVI | CGATTAAGGTTGCAGTCAACCATCGCAA |
| | | | LDFSFLQTLPT | GTTAAAAAATCGCTTGGGTGCATATCATG |
| | | | AQVRNGMAE | CTCCTTTGAAAGTCATCCTCGATTTCTCCT |
| | | | LVKIAVVANS | TCTTGCAAACATTACCAACGGCTCAAGTT |
| | | | EVFELLYEYGE | CGTAATGGGATGGCAGAGTTGGTCAAAA |
| | | | ELLSTHFGYV | TTGCTGTTGTGGCGAACTCTGAAGTCTTT |
| | | | NGTKELKAIA | GAATTGTTGTATGAATATGGCGAAGAGTT |
| | | | HKLNYEAIKT | GCTTTCCACTCACTTTGGATATGTGAATG |
| | | | MLELETPNLH | GTACAAAGGAACTGAAAGCGATCGCACA |
| | | | ELDLDRVIAY | TAAACTCAATTACGAGGCAATAAAAACT |
| | | | GHTWSPTLEL | ATGCTGGAGTTGGAAACTCCAAACTTGC |
| | | | APMIPLFHGHA | ATGAGTTAGACCTCGATCGCGTCATTGCC |
| | | | VNIDMALSATI | TACGGTCACACTTGGAGTCCGACCTTAGA |
| | | | AARRGYITSGE | ATTAGCTCCGATGATACCGTTGTTTCACG |
| | | | RDRILSLMSRI | GTCATGCCGTCAATATAGACATGGCTTTG |
| | | | GLSIDHPLLDG | TCTGCAACTATTGCGGCAAGACGTGGCTA |
| | | | DLLWYATQSIS | CATTACATCAGGAGAACGCGATCGCATTT |
| | | | LTRDGKQRAA | TGAGCTTGATGAGTCGTATAGGTTTATCA |
| | | | MPKPIGECFFV | ATCGATCATCCTCTACTAGATGGCGATTT |
| | | | NDFTREELDA | GCTCTGGTATGCTACCCAATCTATTAGCT |
| | | | ALAEHKRLCA | TGACGCGAGACGGGAAACAACGCGCAGC |
| | | | TYPRGGDGID | TATGCCTAAACCCATTGGTGAGTGCTTCT |
| | | | AYIETQEESKL | TTGTCAACGATTTCACCCGTGAAGAACTA |
| | | | LGV (SEQ ID | GATGCAGCTTTAGCTGAACACAAACGTCT |
| | | | NO: 60) | GTGTGCTACATACCCTCGTGGTGGAGATG |
| | | | | GCATTGACGCTTACATAGAAACTCAAGA |
| | | | | AGAATCCAAACTATTGGGAGTGTGA (SEQ |
| | | | | ID NO: 61) |
| O0-MT | | ACC83904.1 | MTSILGRDTAR | ATGACCAGTATTTTAGGACGAGATACCG |
| | | | PITPHSILVAQL | CAAGACCAATAACGCCACATAGCATTCT |
| | | | QKTLRMAEES | GGTAGCACAGCTACAAAAAACCCTCAGA |
| | | | NIPSEILTSLRQ | ATGGCAGAGGAAAGTAATATTCCTTCAG |
| | | | GLQLAAGLDP | AGATACTGACTTCTCTGCGCCAAGGGTTG |
| | | | YLDDCTTPEST | CAATTAGCAGCAGGTTTAGATCCCTATCT |
| | | | ALTALAQKTSI | GGATGATTGCACTACCCCTGAATCGACCG |
| | | | EDWSKRFSDG | CATTGACAGCACTAGCGCAGAAGACAAG |
| | | | ETVRQLEQEM | CATTGAAGACTGGAGTAAACGCTTCAGT |
| | | | LSGHLEGQTL | GATGGTGAAACAGTGCGTCAATTAGAGC |
| | | | KMFVHITKAK | AAGAAATGCTCTCAGGACATCTTGAAGG |
| | | | SILEVGMFTGY | ACAAACACTAAAGATGTTTGTGCATATCA |
| | | | SALAMAEALP | CTAAGGCTAAAAGCATCCTAGAAGTGGG |
| | | | DDGRLIACEV | AATGTTCACAGGATATTCAGCTTTGGCAA |
| | | | DSYVAEFAQT | TGGCAGAGGCGTTACCAGATGATGGGCG |
| | | | CFQESPHGRKI | ACTGATTGCTTGTGAAGTAGACTCCTATG |
| | | | VVEVAPALET | TGGCCGAGTTTGCTCAAACTTGCTTTCAA |
| | | | LHKLVAKKES | GAGTCTCCCCACGGCCGCAAGATTGTTGT |
| | | | FDLIFIDADKK | AGAAGTGGCACCTGCACTAGAGACGCTG |
| | | | EYIEYFQIILDS | CACAAGCTGGTGGCTAAAAAAGAATCCT |
| | | | HLLAPDGLICV | TTGATCTGATCTTCATTGATGCGGATAAA |
| | | | DNTLLQGQVY | AAGGAGTATATAGAATACTTCCAAATTAT |
| | | | LPSEQRTANGE | CTTGGATAGCCATTTACTAGCTCCCGACG |
| | | | AIAQFNRIVAA | GATTAATCTGTGTAGATAATACTTTGTTG |
| | | | DPRVEQVLLPI | CAAGGACAAGTTTACCTGCCATCAGAAC |
| | | | RDGITLIRRLV | AGCGTACAGCCAATGGTGAAGCGATCGC |
| | | | (SEQ ID | TCAATTCAACCGCATTGTCGCCGCAGATC |
| | | | NO: 62) | CTCGTGTAGAGCAAGTTCTGTTACCCATA |
| | | | | CGAGATGGTATAACCCTGATTAGACGCTT |
| | | | | GGTATAA (SEQ ID NO: 63) |
| AATP- | | ACC83903.1 | MAQSISLSLPQ | ATGGCACAATCAATCTCTTTATCTCTTCCT |
| grasp | | | STTPSKGVRLK | CAATCCACAACGCCATCAAAGGGTGTGA |
| ligase | | | IAALLKTIGTLI | GGCTAAAAATAGCAGCTCTACTGAAGAC |
| | | | LLLIALPLNALI | TATCGGGACTCTAATACTACTGCTGATAG |
| | | | VLISLMCRPFT | CCTTGCCGCTTAATGCTTTGATAGTATTG |
| | | | KKPAVATHPQ | ATATCTCTGATGTGTAGGCCGTTTACAAA |
| | | | NILVSGGKMT | AAAACCTGCCGTAGCCACTCATCCCCAG |
| | | | KALQLARSFH | AATATCTTGGTCAGTGGCGGCAAAATGA |
| | | | AAGHRVILIEG | CCAAAGCATTGCAACTTGCCCGCTCCTTC |
| | | | HKYWLSGHRF | CATGCAGCCGGACACAGAGTTATTCTGAT |
| | | | SNSVSRFYTVP | TGAAGGTCATAAATACTGGTTATCAGGG |
| | | | APQDDPEGYT | CATAGATTCTCAAATTCTGTGAGTCGTTT |
| | | | QALLEIVKREK | TTATACAGTTCCTGCACCACAAGACGACC |
| | | | IDVYVPVCSPV | CAGAAGGCTATACCCAAGCGCTATTGGA |
| | | | ASYYDSLAKS | AATTGTCAAACGAGAGAAGATTGACGTT |
| | | | ALSEYCEVFHF | TATGTACCCGTATGCAGCCCTGTAGCTAG |
| | | | DADITKMLDD | TTATTACGACTCTTTGGCAAAGTCTGCAC |
| | | | KFAFTDRARSL | TATCAGAATATTGTGAGGTTTTTCACTTT |
| | | | GLSAPKSFKIT | GATGCTGATATAACCAAGATGCTGGATG |
| | | | DPEQVINFDFS | ATAAATTTGCCTTTACCGATCGGGCGCGA |
| | | | KETRKYILKSIS | TCGCTTGGTTTATCAGCCCCGAAATCTTT |
| | | | YDSVRRLNLT | TAAAATTACCGATCCTGAACAAGTTATCA |
| | | | KLPCDTPEETA | ACTTCGATTTTAGTAAAGAGACGCGCAA |
| | | | AFVKSLPISPE | ATATATTCTTAAGAGTATTTCTTACGACT |
| | | | KPWIMQEFIPG | CAGTTCGCCGCTTAAATTTAACCAAACTT |
| | | | KELCTHSTVR | CCTTGTGATACCCCAGAAGAGACAGCAG |
| | | | DGELRLHCCS | CGTTTGTCAAGAGTTTACCCATCAGCCCA |
| | | | NSSAFQINYEN | GAAAAACCTTGGATTATGCAAGAATTTAT |
| | | | VENPQIQEWV | TCCTGGGAAAGAATTATGCACCCATAGC |
| | | | QHFVKSLRLT | ACAGTCCGAGACGGCGAATTAAGGTTGC |
| | | | GQISLDFIQAE | ATTGCTGTTCAAATTCTTCAGCGTTTCAG |
| | | | DGTAYAIECNP | ATTAACTATGAAAATGTCGAAAATCCCC |
| | | | RTHSAITMFYN | AAATTCAAGAATGGGTACAACATTTCGTC |
| | | | HPGVAEAYLG | AAAAGTTTACGGCTGACTGGACAAATAT |
| | | | KTPLAAPLEPL | CTCTTGACTTTATCCAAGCTGAAGATGGT |
| | | | ADSKPTYWIY | ACAGCTTATGCCATTGAATGTAATCCTCG |
| | | | HEIWRLTGIRS | CACCCATTCGGCGATCACAATGTTCTACA |
| | | | GQQLQTWFGR | ATCACCCAGGTGTTGCAGAAGCCTATCTT |
| | | | LVRGTDAIYRL | GGTAAAACTCCTCTAGCTGCACCTTTGGA |
| | | | DDPIPFLTLHH | ACCTCTTGCAGATAGCAAGCCCACTTACT |
| | | | WQITLLLLQNL | GGATATATCACGAAATCTGGCGATTGACT |
| | | | QRLKGWVKID | GGGATTCGCTCTGGACAACAATTACAAA |
| | | | FNIGKLVELGG | CTTGGTTTGGGAGATTAGTCAGAGGTACA |
| | | | DYSRSEE (SEQ | GATGCCATTTATCGCCTGGACGATCCGAT |
| | | | ID NO: 64) | ACCATTTTTAACTTTGCACCATTGGCAGA |
| | | | | TTACTTTACTTTTGCTACAAAATTTGCAA |
| | | | | CGACTCAAAGGTTGGGTAAAGATCGATT |
| | | | | TTAATATCGGTAAACTCGTGGAATTAGGG |
| | | | | GGCGACTACTCGAGGTCTGAAGAATGA |
| | | | | (SEQ ID NO: 65) |
| DD-ala- | | ACC83902.1 | MPVLNILHLV | ATGCCAGTACTTAATATCCTTCATTTAGT |
| D-ala | | | GSAHDKFYCD | TGGGTCTGCACACGATAAGTTTTACTGTG |
| ligase | | | LSRLYAQDCL | ATTTATCACGTCTTTATGCCCAAGACTGT |
| | | | AATADPSLYN | TTAGCTGCAACAGCAGATCCATCGCTTTA |
| | | | FQIAYITPDRQ | TAACTTTCAAATTGCATATATCACACCCG |
| | | | WRFPDSLSRE | ATCGGCAGTGGCGATTTCCTGACTCTCTC |
| | | | DIALTKPIPVFD | AGTCGAGAAGATATTGCTCTTACCAAACC |
| | | | AIQFLTGQNID | GATTCCTGTGTTTGATGCCATACAATTTC |
| | | | MMLPQMFCIP | TAACAGGCCAAAACATTGACATGATGTT |
| | | | GMTQYRALFD | ACCACAAATGTTTTGTATTCCTGGAATGA |
| | | | LLKIPYIGNTP | CTCAGTACCGTGCCCTATTCGATCTGCTC |
| | | | DIMAIAAHKA | AAGATCCCTTATATAGGAAATACCCCAG |
| | | | RAKAIVEAAG | ATATTATGGCGATCGCGGCCCACAAAGC |
| | | | VKVPRGELLR | CAGAGCCAAAGCAATTGTCGAAGCAGCA |
| | | | QGDIPTITPPAV | GGGGTAAAAGTGCCTCGTGGAGAATTGC |
| | | | VKPVSSDNSL | TTCGCCAAGGAGATATTCCAACAATTACA |
| | | | GVVLVKDVTE | CCTCCAGCAGTCGTCAAACCTGTAAGTTC |
| | | | YDAALKKAFE | TGACAACTCTTTAGGAGTAGTCTTAGTTA |
| | | | YASEVIVEAFI | AAGATGTGACTGAATATGATGCTGCCTTA |
| | | | ELGREVRCGII | AAGAAAGCATTTGAATATGCTTCGGAGG |
| | | | VKDGELIGLPL | TCATCGTAGAAGCATTCATCGAACTTGGT |
| | | | EEYLVDPHDK | CGAGAAGTCAGATGCGGCATCATTGTAA |
| | | | PIRNYADKLQ | AAGACGGTGAGCTAATAGGTTTACCCCTT |
| | | | QTDDGDLHLT | GAAGAGTATCTGGTAGACCCACACGATA |
| | | | AKDNIKAWIL | AACCTATCCGTAACTATGCTGATAAACTC |
| | | | DPNDPITQKVQ | CAACAAACTGACGATGGCGACTTGCATTT |
| | | | QVAKRCHQAL | GACTGCTAAAGATAATATCAAGGCTTGG |
| | | | GCRHYSLFDF | ATTTTAGACCCTAACGACCCAATCACCCA |
| | | | RIDPKGQPWFL | AAAGGTTCAGCAAGTGGCTAAAAGGTGT |
| | | | EAGLYCSFAP | CATCAGGCTTTGGGTTGTCGCCACTACAG |
| | | | KSVISSMAKA | TTTATTTGACTTCCGAATCGATCCAAAGG |
| | | | AGIPLNDLLIT | GACAACCTTGGTTCTTAGAAGCTGGATTA |
| | | | AINETLGSNKK | TATTGTTCTTTTGCCCCCAAAAGTGTGAT |
| | | | VLQN (SEQ ID | TTCTTCTATGGCGAAAGCAGCCGGAATCC |
| | | | NO: 66) | CTCTAAATGATTTATTAATAACCGCTATT |
| | | | | AATGAAACATTGGGTAGTAATAAAAAGG |
| | | | | TGTTACAAAATTGA (SEQ ID NO: 67) |
| NNRPS | Anabaena | ABA23460.1 | MQTIDFNIRKL | ATGCAGACTATAGATTTTAATATTCGTAA |
| | variabilis | | LVEWNATHRD | GTTACTTGTAGAGTGGAACGCGACCCAC |
| | | | YDLSQSLHELI | AGAGATTATGATCTTTCCCAGAGTTTACA |
| | | | VAQVERTPEAI | TGAACTAATTGTAGCTCAAGTAGAACGA |
| | | | AVTFDKQQLT | ACACCTGAGGCGATCGCTGTCACCTTTGA |
| | | | YQELNHKANQ | CAAGCAACAACTAACTTATCAAGAACTA |
| | | | LGHYLQTLGV | AATCATAAAGCAAACCAGCTAGGACATT |
| | | | QPETLVGVCL | ATTTACAAACATTAGGAGTCCAGCCAGA |
| | | | ERSLEMVICLL | AACCCTGGTAGGCGTTTGTTTAGAACGTT |
| | | | GILKAGGAYV | CCTTAGAAATGGTTATCTGTCTTTTAGGA |
| | | | PIDPEYPQERIA | ATCCTCAAAGCTGGGGGTGCTTATGTTCC |
| | | | YMLEDSQVKV | TATTGACCCTGAATATCCTCAAGAACGCA |
| | | | LLTQEKLLNQI | TAGCTTATATGCTAGAAGATTCTCAGGTG |
| | | | PHHQAQTICV | AAGGTACTACTAACTCAAGAAAAATTAC |
| | | | DREWEKISTQ | TCAATCAAATTCCCCACCATCAAGCACAA |
| | | | ANTNPKSNIKT | ACTATCTGTGTAGATAGGGAATGGGAGA |
| | | | DNLAYVIYTS | AAATTTCCACACAAGCTAATACCAATCCC |
| | | | GSTGKPKGAM | AAAAGTAATATAAAAACGGATAATCTTG |
| | | | NTHKGICNRLL | CTTATGTAATTTACACCTCTGGTTCCACT |
| | | | WMQEAYQIDS | GGTAAACCAAAAGGTGCAATGAACACCC |
| | | | TDSILQKTPFSF | ACAAAGGTATCTGTAATCGCTTATTGTGG |
| | | | DVSVWEFFWT | ATGCAGGAAGCTTATCAAATCGATTCCAC |
| | | | LLTGARLVIAK | AGATAGCATTTTACAAAAAACCCCCTTTA |
| | | | PGGHKDSAYLI | GTTTTGATGTTTCCGTTTGGGAGTTCTTTT |
| | | | DLITQEQITTL | GGACTTTATTAACTGGCGCACGTTTGGTA |
| | | | HFVPSMLQVF | ATAGCCAAACCAGGCGGACATAAAGATA |
| | | | LQNRHVSKCS | GTGCTTACCTCATCGATTTAATTACTCAA |
| | | | SLKRVICSGEA | GAACAAATCACTACGTTGCATTTTGTCCC |
| | | | LSIDLQNRFFQ | CTCAATGCTGCAAGTGTTTTTACAAAATC |
| | | | HLQCELHNLY | GCCATGTAAGCAAATGCAGCTCTCTAAA |
| | | | GPTEAAIDVTF | AAGAGTTATTTGTAGCGGTGAAGCTTTAT |
| | | | WQCRKDSNLK | CTATAGATTTACAAAATAGATTTTTCCAG |
| | | | SVPIGRPIANT | CATTTGCAATGTGAATTACATAACCTCTA |
| | | | QIYILDADLQP | TGGCCCGACAGAAGCAGCAATTGATGTC |
| | | | VNIGVTGEIYI | ACATTTTGGCAATGTAGAAAAGATAGTA |
| | | | GGVGVARGYL | ATTTAAAGAGTGTACCTATTGGTCGTCCC |
| | | | NKEELTKEKFII | ATTGCTAATACTCAAATTTATATTCTTGA |
| | | | NPFPNSEFKRL | TGCCGATTTACAACCAGTAAATATTGGTG |
| | | | YKTGDLARYL | TCACTGGTGAAATTTATATTGGTGGTGTA |
| | | | PDGNIEYLGRT | GGGGTTGCTCGTGGTTATTTGAATAAAGA |
| | | | DYQVKIRGYRI | AGAATTGACCAAAGAAAAATTTATTATT |
| | | | EIGEIENVLSSH | AATCCCTTTCCCAATTCTGAGTTTAAGCG |
| | | | PQVREAVVIA | ACTTTATAAAACAGGTGATTTAGCTCGTT |
| | | | RDDNAQEKQII | ATTTACCCGATGGAAATATTGAATATCTT |
| | | | AYITYNSIKPQ | GGTAGAACAGATTATCAAGTAAAAATTC |
| | | | LDNLRDFLKA | GGGGTTATAGAATTGAAATTGGCGAGAT |
| | | | RLPDFMIPAAF | TGAAAATGTTTTATCTTCACACCCACAAG |
| | | | VMLEHLPLTPS | TCAGAGAAGCTGTAGTCATAGCGCGGGA |
| | | | GKVDRKALPK | TGATAACGCTCAAGAAAAACAAATCATC |
| | | | PDLFNYSEHNS | GCTTATATTACCTATAACTCCATCAAACC |
| | | | YVAPRNEVEE | TCAGCTTGATAATCTGCGTGATTTCCTAA |
| | | | KLVQIWSNILH | AAGCAAGGCTACCTGATTTTATGATTCCA |
| | | | LPKVGVTENFF | GCCGCTTTTGTGATGCTGGAGCATCTTCC |
| | | | AIGGNSLKAL | TTTAACTCCCAGTGGTAAAGTAGACCGTA |
| | | | HLISQIEELFAK | AGGCATTACCTAAGCCTGATTTATTTAAT |
| | | | EISLATLLTNP | TATAGTGAACATAATTCCTATGTAGCGCC |
| | | | VIADLAKVIQA | TCGGAATGAAGTTGAAGAAAAATTAGTA |
| | | | NNQIHNSPLVP | CAAATCTGGTCGAATATTCTGCATTTACC |
| | | | IQPQGKQQPFF | TAAAGTAGGTGTGACAGAAAACTTTTTCG |
| | | | CIHPAGGHVL | CTATTGGTGGTAATTCCCTCAAAGCTCTA |
| | | | CYFKLAQYIGT | CATTTAATTTCTCAAATTGAAGAGTTATT |
| | | | DQPFYGLQAQ | TGCTAAAGAGATATCCTTAGCAACACTTT |
| | | | GFYGDEAPLT | TAACAAATCCAGTAATTGCAGATTTAGCC |
| | | | RVEDMASLYV | AAGGTTATTCAAGCAAACAACCAAATCC |
| | | | KTIREFQPQGP | ATAATTCACCCCTAGTTCCAATTCAACCA |
| | | | YRVGGWSFGG | CAAGGTAAGCAGCAGCCTTTCTTTTGTAT |
| | | | VVAYEVAQQL | ACATCCTGCTGGTGGTCATGTTTTATGCT |
| | | | HRQGQEVSLL | ATTTTAAACTCGCACAATATATAGGAACT |
| | | | AILDSYVPILL | GACCAACCATTTTATGGCTTACAAGCTCA |
| | | | DKQKPIDDVY | AGGATTTTATGGAGATGAAGCACCCTTG |
| | | | LVGVLSRVFG | ACGCGAGTTGAAGATATGGCTAGTCTCTA |
| | | | GMFGQDNLVT | CGTCAAAACTATTAGAGAATTTCAACCCC |
| | | | PEEIENLTVEE | AAGGGCCTTATCGTGTCGGGGGGTGGTC |
| | | | KINYIIDKARS | ATTTGGTGGAGTCGTAGCTTATGAAGTAG |
| | | | ARIFPPGVERQ | CACAGCAGTTACATAGACAAGGACAAGA |
| | | | NNRRILDVLV | AGTATCTTTACTAGCAATATTAGATTCTT |
| | | | GTLKATYSYIR | ACGTACCGATTCTGCTGGATAAACAAAA |
| | | | QPYPGKVTVF | ACCCATTGATGACGTTTATTTAGTTGGTG |
| | | | RAREKHIMAP | TTCTCTCCAGAGTTTTTGGCGGTATGTTTG |
| | | | DPTLVWVELF | GTCAAGATAATCTAGTCACACCTGAAGA |
| | | | SVMAAQEIKII | AATAGAAAATTTAACTGTAGAAGAAAAA |
| | | | DVPGNHYSFV | ATTAATTACATCATTGATAAAGCACGGA |
| | | | LEPHVQVLAQ | GCGCTAGAATATTCCCGCCTGGTGTAGAA |
| | | | RLQDCLENNS | CGTCAAAATAATCGCCGTATTCTTGATGT |
| | | | (SEQ ID NO: 68) | TTTGGTGGGAACTTTAAAAGCAACTTATT |
| | | | | CCTATATAAGACAACCATATCCAGGAAA |
| | | | | AGTCACTGTATTTCGAGCCAGGGAAAAA |
| | | | | CATATTATGGCTCCTGACCCGACCTTAGT |
| | | | | TTGGGTAGAATTATTTTCTGTAATGGCGG |
| | | | | CTCAAGAAATTAAGATTATTGATGTCCCT |
| | | | | GGAAACCATTATTCGTTTGTTCTAGAACC |
| | | | | CCATGTACAGGTTTTAGCACAGCGTTTAC |
| | | | | AAGATTGTCTGGAAAATAATTCATAA |
| | | | | (SEQ ID NO: 69) |

It is apparent that any protein which has an amino acid sequence with deletion, modification, substitution, or addition in part of the sequence can also be comprised within the scope of the present disclosure, as long as the amino acid sequence has a homology or identity to the SEQ ID NOS described herein and has a biological activity substantially identical or corresponding to the enzyme proteins of the SEQ ID NOS described above.

Considering the codons preferred in an organism, where the protein (enzyme) is to be expressed, due to codon degeneracy, various modifications may be performed in the coding region of the nucleotide sequence within the scope not altering the amino acid sequence of the protein to be expressed from the coding region. Therefore, the mycosporine-like amino acid biosynthesis gene can be comprised without limitation as long as it is a nucleic acid sequence encoding a protein involved in mycosporine-like amino acid biosynthesis.

Alternatively, any sequence which encodes a protein involved in the biosynthesis of mycosporine-like amino acids by hybridizing with any probe that can be prepared from known gene sequences (e.g., complementary sequences to all or part of the above polynucleotide sequence) under stringent conditions, may be comprised without limitation.

All steps described herein, for example, the enhancement of an enzyme activity and/or the introduction of genes may be performed in a simultaneous, sequential, and reverse order regardless of the order.

The microorganism producing mycosporine-like amino acids can produce mycosporine-like amino acids by comprising a mycosporine-like amino acid biosynthesis enzyme, a gene encoding the same, or a cluster of said gene, and additionally, may be a microorganism in which the mycosporine-like amino acid-producing ability is increased by comprising the above-described xylose assimilation enzyme, and/or by enhancing the pentose phosphate pathway. In particular, the microorganism producing the mycosporine-like amino acids may use xylose as a carbon source, and may produce mycosporine-like amino acids from xylose.

The microorganism can be selected without limitation among microorganisms capable of producing mycosporine-like amino acids by comprising a mycosporine-like amino acid biosynthetic enzyme, comprising the above-mentioned xylose assimilation enzyme, and/or performing mutation so as to enhance the pentose phosphate pathway. For example, the microorganism may be a yeast, a microorganism of the genus Corynebacterium, or a microorganism of the genus Escherchia.

The yeast may be at least one selected from the group consisting of Saccharomycotina and Taphrinomycotina of the phylum Ascomycora, Agancomycotina of the phylum Basidiomycota, a microorganism belonging to the phylum Pucciniomycoina, etc., specifically at least one selected from the group consisting of a microorganism of the genus Saccharomyces, a microorganism of the genus Schizosaccharomyces, a microorganism of the genus Phaffia, a microorganism of the genus Kluyveromyces, a microorganism of the genus Pichia, and a microorganism of the genus Candida, and more specifically Saccharomyces cerevisiae, but the microorganism is not limited thereto.

The microorganism of the genus Corynebacterium may be at least one selected from the group consisting of Corynebacteriun glutamwun, Corynebacterium ammoniagenes, Brevibacterium lactofermentum, Brevibactenum flavum, Corynebacteriun thermoaminogenes, Corynebacterium efficiens, etc., and specifically may be Corynebacterium glutamicum, but the microorganism is not limited thereto.

The microorganism of the genus Escherichia may be at least one selected from the group consisting of Escherichia albertii, Escherichia coli, Escherichia fergusonmi, Escherichia hermannii, Escherichia vulneris, etc., and specifically may be Escherichia coli, but the microorganism is not limited thereto.

In one specific embodiment, the microorganism may be an yeast. Yeast (Saccharomyces cerevisiae) is a promising strain for the production of natural products for which safe and diverse genetic manipulation techniques have been reported. For example, the inflow of S7P must be smooth in order to introduce a shinorine-producing gene derived from Nostoc punctiforme or Anabaena variabilis in yeast to increase shinorine production. When yeast is cultured using glucose as a carbon source, most of the carbon source is converted to pyruvate via glycolysis. Therefore, when glucose is used as a single carbon source, it is difficult to enhance the pentose glucose pathway and therefore, the inflow amount of S7P cannot be increased. When xylose is used as a carbon source, the production of S7P increases as it flows into the pentose pathway via anabolism. Yeast cannot use xylose naturally, but xylose can be utilized by introducing a heterogeneous pathway consisting of xylose isomerase (XI) or xylose reductase (XR) and/or xylitol dehydrogenase (XDH). XI is an enzyme that degrades xylose in most bacteria and has the advantage of being able to eliminate cofactor imbalance during xylose fermentation. However, considering the enzyme activity when expressed in yeast, the XR/XDH pathway can be used for xylose fermentation in yeast. Xylose reductase (XR) and xylitol dehydrogenase (XDH) sequentially convert xylose to xylitol and xylitol to xylulose. Xylulose is converted to xylulose-5-phosphate by xylulokinase (XK), and then, xylulose-5-phosphate flows into the pentose phosphate pathway (FIG. 1). Therefore, the use of xylose as a carbon source can increase S7P influx via the pentose phosphate pathway. Additionally, by expressing xylose assimilation genes encoding XR(Xyl1), XDH(Xyl2) and XK(Xyl3) of Pichia stipitis, a xylose fermenting yeast, in S. cerevisiae, thereby proposing that S. cerevisiae strains having xylose fermentation ability can be constructed.

Introduction, Insertion, or Mutation of a Gene

The introduction of a gene described herein can be performed by i) introducing a foreign gene (homologous and/or heterologous to the microorganism (host cell) into the host cell in a form operably linked to a vector, or ii) by inserting (e.g., randomly inserting) the foreign gene into a chromosome (genome) of the host cell. ii) When inserted into the chromosome (genome) of the host cell, the insertion position may be a position that does not affect the growth of the host cell (e.g., a non-transcriptional spacer (NTS), etc.) and/or a position that can increase random insertion efficiency (e.g., retrotransposon, etc.), but the position is not limited thereto.

The introduction of a gene or vector can be performed by appropriately selecting a known transformation method by those skilled in the art, and for example, it may be performed by electroporation, lipofection, microinjection, particle bombardment, polyethylene glycol-mediated uptake, etc., but the method is not limited thereto. For example, the introduction can be performed by using at least one selected from the group consisting of RNA-guided endonuclease system (e.g., (a) an RNA-guided endonuclease (e.g., Cas9 protein, etc.), a gene encoding the same, or a vector containing the gene; and (b) guide RNA (e.g., single guide RNA (sgRNA), etc.), a mixture containing the encoding DNA thereof, or a vector containing the DNA (e.g., a mixture of RNA-guided endonuclease protein and guide RNA, etc.), a complex (e.g., ribonucleic acid protein (RNP) in which RNA-guided endonuclease protein and guide RNA am fused, recombinant vector (e.g., vector containing RNA-guided endonuclease encoding gene and guide RNA encoding DNA, etc.), and the like.

Production of Mycosporine-Like Amino Acids

Still another embodiment provides a method for producing mycosporine-like amino acids, comprising a step of culturing the mutant microorganism. The production method may further comprise, after the culturing, recovering mycosporine-like amino acids from the cultured microorganism, medium, or a combination thereof.

The “microorganism” and “mycosporine-like amino acid” are as described above.

The “cultivation” means growing the microorganism under moderately controlled environmental conditions.

As used herein, the term “culture” means that the microorganism is grown under appropriately controlled environmental conditions. The culture process can be performed in a suitable culture medium and culture conditions known in the art. Such a culture process may be easily adjusted for use by those skilled in the art according to the microorganism to be selected. The step of culturing the microorganism can be performed in batch culture, continuous culture, fed-batch culture, etc. known in the art, but the step of culturing the microorganism is not particularly limited thereto. The medium and other culture conditions used for culturing the microorganism can be used without particular limitation as long as if it is a medium used in the conventional culture for a microorganism. For example, the microorganism described herein may be cultured under aerobic conditions in a conventional medium containing an appropriate carbon source, nitrogen source, phosphorous source, inorganic compound, amino acid, and/or vitamin, etc. while adjusting temperature, pH, etc. Specifically, the pH may be adjusted using a basic compound (e.g., sodium hydroxide, potassium hydroxide, or ammonia) or an acidic compound (e.g., phosphoric acid or sulfuric acid) so as to obtain an optimal pH (e.g., pH 5 to 9, specifically pH 6 to 8, and most specifically pH 6.8), bit the method of pH adjustment is not limited thereto. Additionally, oxygen or oxygen-containing gas may be injected into the culture in order to maintain an aerobic state of the culture; or nitrogen, hydrogen, or carbon dioxide gas may be injected into the culture without gas injection in order to maintain an anaerobic or microaerobic state of the culture, but the gas is not limited thereto. Additionally, the culture temperature may be maintained at 20 to 45° C., and specifically 25 to 40° C., and the culture may be performed for about 10 hours to about 160 hours, without being limited thereto. Additionally, during the culture, an antifoaming agent (e.g., fatty acid polyglycol ester) may be added to prevent foam generation, but is not limited thereto.

As a carbon source to be used in the medium for culture, saccharides and carbohydrates (e.g., glucose, sucrose, lactose, fructose, maltose, molasses, starch, and cellulose), oils and fats (e.g., soybean oil, sunflower oil, peanut oil, and coconut oil), fatty acids (e.g., palmitic acid, stearic acid, and linoleic acid), alcohols (e.g., glycerol and ethanol), organic acids (e.g., acetic acid), etc. may be used alone or in combination, but the carbon source is not limited thereto. As a nitrogen source, a nitrogen-containing organic compound (e.g., peptone, yeast extract, meat gravy, malt extract, corn steep liquor, bean flour, and urea), or an inorganic compound (e.g., ammonium sulfate, ammonium chloride, ammonium phosphate, ammonium carbonate, and ammonium nitrate), etc. may be used alone or in combination, but the nitrogen source is not limited thereto. As a phosphorous source, potassium dihydrogen phosphate, dipotassium hydrogenphosphate, and sodium-containing salts corresponding thereto may be used alone or in combination, but the phosphorous source is not limited thereto. Additionally, the medium may comprise essential growth-promoting materials, such as a metal salt (e.g., magnesium sulfate or iron sulfate), amino acids, and vitamins.

In one embodiment, the medium may essentially contain xylose as a carbon source. For example, the medium may comprise xylose and other sugars, for example, glucose, as carbon sources. When the medium contains xylose and glucose as carbon sources, the content ratio between xylose and glucose as a carbon source in the medium may be in the range of 1:9 to 9:1 (xylose content:glucose content), 2:8 to 5:5, 3:7 to 5:5, or 4:6 to 5:5 on a weight basis, in consideration of the degree of mycosporine-like amino acid production, the degree of microbial growth, and/or the degree of by-product production.

The mycosporine-like amino acids produced by culturing may be secreted into the medium or remain in the cells.

As used herein, the term “medium” refers to a culture medium for culturing the microorganism and/or a product obtained after culture. The medium is a concept, which comprises both a form where the microorganism is comprised and a form where the microorganism is removed from the microorganism-containing cultured solution by centrifugation, filtration, etc.

In the step of recovering the mycosporine-like amino acids produced in the culturing step, the desired MAAs can be collected from the culture solution using an appropriate method known in the art according to the culture method. For example, the step can be performed by using centrifugation, filtration, anion-exchange chromatography, crystallization, HPLC, etc. and the desired mycosporine-like amino acids can be recovered from the cultured microorganism or medium using an appropriate method known in the art. The step of recovering the mycosporine-like amino acids may further comprise a separation step and/or a purification step.

### Advantageous Effects

The microorganism provided herein is a strain capable of producing mycosporine-like amino acids by consuming xylose as a carbon source, has improved mycosporine-like amino acid-producing ability, and can be efficiently used for producing mycosporine-like amino acids through fermentation utilizing lignocellulosic biomass.

### BRIEF DESCRIPTION OF THE DRAWINGS

FIG. 1a is a schematic diagram exemplarily showing the shinorine biosynthesis pathway among mycosporine-like amino acids.

FIG. 1b shows the mycosporine-like amino acid biosynthesis gene clusters of Nostoc punctiforme and Anabaena variabilis.

FIG. 2a is a graph showing the results of HPLC analysis of the cell extract after culturing yeast strain JHYS10 introduced with plasmids containing DDGS (NpR5600), O-MT (NpR5599), ATP-grasp ligas (NpR5598), and D-ala-D-ala ligase (NpR5597) genes for 48 hours.

FIG. 2b is a graph showing the synorin production results after culturing the yeast strain JHYS10 for 48 hours in comparison with the wild-type strain (WT-1).

FIG. 2c is a graph showing the cell growth during the culture of the yeast strain JHYS10 for 48 hours in comparison with the wild-type strain (WT-1).

FIGS. 3a and 3b are graphs showing the results of tandem mass spectrometry analysis of cell extracts after culturing the yeast strain JHYS10 for 48 hours.

FIG. 4a is a cleavage map of a vector comprising an expression cassette containing D-ala-D-ala ligase (NpR5597) and DDGS (NpR5600) genes, and an expression cassette containing ATP-grasp ligase (NpR5598) and O-MT (NpR5599) genes.

FIG. 4b is a graph showing the results of shinorine production of yeast strains JHYS12 and JHYS13 in which the genes were randomly inserted by transformation with the two expression cassettes of FIG. 4a.

FIG. 4c is a graph showing the copy number of each gene in yeast strains JHYS11, JHYS12, and JHYS13 in which the genes were randomly inserted by transformation with the two expression cassettes of FIG. 4a.

FIG. 5 is a graph showing the consumption of glucose and/or xylose, the degree of cell growth resulting therefrom, and the result of shinorine production, wherein the glucose and/or xylose is obtained by culturing a JHYS13-2 strain in which a plasmid containing the xylose assimilation genes XYL1, XYL2, and XYL3 genes is introduced into the yeast strain JHYS13, in a medium containing xylose and glucose.

FIG. 6a is a cleavage map of a vector for constructing a yeast strain in which a gene encoding a xylose assimilation enzyme is inserted into the chromosome by NTS.

FIG. 6b is a graph showing the consumption of glucose and/or xylose, and the degree of cell growth resulting therefrom, which is obtained by culturing yeast strains JHYS14, JHYS15, and JHYS16 in which the xylose assimilation genes XYL1, XYL2, and XYL3 genes are randomly inserted into the chromosome of the yeast strain JHYS13, in a medium containing xylose and glucose.

FIG. 6c is a graph showing the results of shinorine production of yeast strains JHYS14, JHYS15, and JHYS16.

FIG. 7 is a graph showing the consumption of glucose and/or xylose, the degree of cell growth resulting therefrom, and the result of shinorine production, which is obtained by culturing a JHYS17 strain in which the TAL1 gene is deleted in the JHYS16 strain, in a medium containing xylose and glucose.

FIG. 8a is a graph showing the consumption of glucose and/or xylose and the degree of cell growth resulting therefrom, which is obtained by culturing a JHYS17-3 strain into which the TKL1 gene was introduced into the JHYS17 strain, and a JHYS17-4 strain into which both the STB5 gene and the TKL1 gene were introduced, in a medium containing xylose and glucose.

FIG. 8b is a graph showing the results of shinorine production obtained by culturing the JHYS17-2 strain, the JHYS17-3 strain, and the JHYS17-4 strain in a medium containing xylose and glucose.

FIG. 9a is a graph showing the consumption of glucose and/or xylose and the degree of cell growth resulting therefrom, which is obtained by culturing a JHYS17-4 strain in a medium containing xylose and glucose in various ratios.

FIGS. 9b and 9c are graphs showing the results of shinorine production, which is obtained by culturing a JHYS17-4 strain in a medium containing xylose and glucose in various ratios.

FIG. 10 is a graph showing the production of by-products (xylitol, ethanol, glycerol, and acetate), which is obtained by culturing a JHYS17-4 strain in a medium containing xylose and glucose in various ratios.

### DETAILED DESCRIPTION OF THE EMBODIMENTS

Hereinafter, the present disclosure will be described in more detail with reference to the following Examples. However, these Examples are for illustrative purposes only and are not intended to limit the scope of the disclosure. It will be apparent to those skilled in the art that the Examples described below can be modified without departing from the essential gist of the invention.

<Introduction of Exogenous Mycosporine-Like Amino Add Biosynthesis Pathway Based on Yeast Strain>

### Example 1: Preparation of Vector for Expression in Yeast of Mycosporine-Like Amino Acid Biosyntehsis Gene Derived from Microalgae

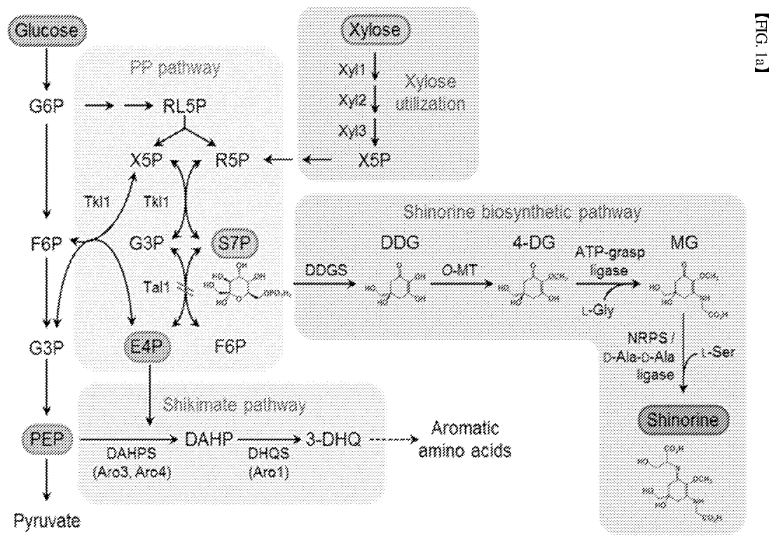

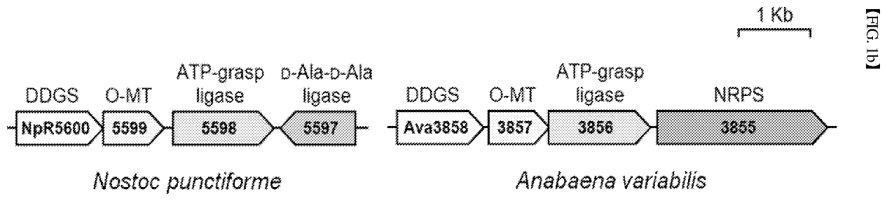

In order to use yeast (Saccharomyces cerevisiae) as a mycosporine-like amino acid production strain, a mycosporine-like amino acid biosynthesis gene derived from Nostoc punctiforme (ATCC 29133), which is one of cyanobacteria, was introduced into a vector for yeast expression. N. punctiforme shinorine biosynthesis involves four enzymatic reactions: 2-dimethyl 4-deoxygadusol synthase (DDGS), O-methyltransferase (O-MT), ATP-grasp ligase, and D-ala-D-ala ligase which use sedoheptulose 7-phosphate (S7P) as a substrate (see FIG. 1a). The genes encoding the four enzymes in the N. punctiforme genome are as follows, respectively (see the left of FIG. 1b): DDGS: NpR5600 (GenBanK Accession No. ACC83905.1; REGION: 6913123 . . . 6914355), O-MT: NpR5599 (GenBanK Accession No. ACC83904.1; REGION: 6912285 . . . 6913118), ATP-grasp ligase: NpR5598 (GenBanK Accession No. ACC83903.1; REGION: 6910776 . . . 6912161), and D-ala-D-ala ligase: NpR5597 (GenBanK Accession No. ACC83902.1; REGION: 6909563 . . . 6910609).

The primers used for the construction of the plasmid containing the four genes are summarized in Table 4 below:

TABLE 4

| | | Primer (F: | | SEQ |
|---|---|---|---|---|
| PCR | Template | Forward, R: | | ID |
| Product | used | Reverse) | Sequence(5′→3′) | NO |
| NpR5600 | Nostoc | NpR5600 F | GCGGGATCCATGAGTAATGTTCAAGCATCG | 1 |
| | punctiforme | NpR5600 R | GCGCTCGAGTCACACTCCCAATAGTTTGG | 2 |
| NpR5599 | genomic | NpR5599 F | GCGGGATCCATGACCAGTATTTTAGGACG | 3 |
| | DNA | NpR5599 R | GCGCTCGAGTTATACCAAGCGTCTAATCAG | 4 |
| NpR5598 | (GenBank | NpR5598 F | GCGGGATCCATGGCACAATCAATCTCTTTA | 5 |
| | Accession | NpR5598 R | GCGCTCGAGTAGTCGCCCCCTAATTCC | 6 |
| NpR5597 | No. | NpR5597 F | GCGGGATCCATGCCAGTACTTAATATCCTT | 7 |
| | CP001037.1) | NpR5597 R | GCGCTCGAGTCAATTTTGTAACACCTTTTTATTA | 8 |
| P_{TDH3}- | p413GPD- | Univ F2 | GACTCGCGCGCGGGAACAAAAGCTGGAGCTC | 32 |
| NpR560- | NpR5600 | | GACTACGCGTGCGGCCGCTAATGGCGCGCCATAGGG | 33 |
| T_{CYC1} | | Univ R | CGAATTGGGTACC | |
| P_{TDH3}- | p414GPD- | Univ F2 | GACTCGCGCGCGGGAACAAAAGCTGGAGCTC | 32 |
| NpR5599- | NpR5599 | Univ R | GACTACGCGTGCGGCCGCTAATGGCGCGCCATAGGG | 33 |
| T_{CYC1} | | | CGAATTGGGTACC | |
| P_{TEF1}- | coex414TEF- | Univ F2 | GACTCGCGCGCGGGAACAAAAGCTGGAGCTC | 32 |
| NpR5598- | NpR5598 | Univ R | GACTACGCGTGCGGCCGCTAATGGCGCGCCATAGGG | 33 |
| T_{GPM1} | | | CGAATTGGGTACC | |

The genome of N. punctiforme (GenBank Accession No. CP001037.1) was used as a template, the four gene fragments involved in shinorine biosynthesis were amplified by PCR using the primers, and then they were cleaved with BamHI-XhoI restriction enzymes, and cloned into p413GPD, coex413TEF, p414GPD, and coex414TEF respectively retreated with the same restriction enzyme. The vectors thus constructed were named p413GPD-NpR5600, p414GPD-NpR5599, coex414TEF-NpR5598, and coex413TEF-NpR5597, respectively.

The constructed vector was amplified thy PCR with Univ F2 (SEQ ID NO: 32)-Univ R primer (SEQ ID NO: 33) as a template to secure the [Promoter-ORF-Terminator] gene fragment, and then cleaved with MauBI-NotI restriction enzymes and successively cloned into coex413TEF-NpR5597 vector cleaved with AscI-NotI, whereby four genes were introduced into one vector, which was named coex413-NpR4. The vectors used and the constructed vectors are summarized in Table 5 below:

TABLE 5

| Plasmid | Description |
|---|---|
| coex413TEF | CEN/ARS plasmid, HIS3, P_{TEF1}, |
| | T_{GPM1} |
| coex414TEF | CEN/ARS plasmid, TRP1, P_{TEF1}, |
| | T_{GPM1} |
| p413GPD(ATCC ® 87354 ™) | CEN/ARS plasmid, HIS3, P_{TDH3}, |
| | T_{CYC1} |
| p414GPD(ATCC ® 87356 ™) | CEN/ARS plasmid, TRP1, P_{TDH3}, |
| | T_{CYC1} |
| p414ADH(ATCC ® 87372 ™) | CEN/ARS plasmid, TRP1, P_{ADH1}, |
| | T_{CYC1} |
| p416GPD(ATCC ® 87360 ™) | CEN/ARS plasmid, URA3, P_{TDH3}, |
| | T_{CYC1} |
| p413GPD-NpR5600 | CEN/ARS plasmid, HIS3, |
| | P_{TDH3}-NpR5600-T_{CYC1} |
| p414GPD-NpR5599 | CEN/ARS plasmid, TRP1, |
| | P_{TDH3}-NpR5599-T_{CYC1} |
| coex414TEF-NpR5598 | CEN/ARS plasmid, TRP1, |
| | P_{TEF1}-NpR5598-T_{GPM1} |
| coex413TEF-NpR5597 | CEN/ARS plasmid, HIS3, |
| | P_{TEF1}-NpR5597-T_{GPM1} |
| coex413-NpR4 | CEN/ARS plasmid, HIS3, |
| | P_{TEF1}-NpR5597-T_{GPM1}, |
| | P_{TDH3}-NpR5600-T_{CYC1}, |
| | P_{TEF1}-NpR5598-T_{GPM1}, |
| | P_{TDH3}-NpR5599-T_{CYC1} |

(In Table 5 above, coex413TEF and coex414TEF were constructed from p413TEF (ATCC® 87362) and p414TEF (ATC® 87364), respectively, with reference to the method described in Korean Unexamined Patent Publication No. 10-2016-0093492)

### Example 2: Evaluation of Chlorine-Producing Ability of Strains Through Expression in Vector-Based Yeast of Mycosporine-Like Amino Acid Biosyntehsis Gene

A heterologous mycosporine-like amino acid biosynthesis pathway gene derived from N. punctiforme was transformed into a wild-type S. cerevisiae strain, CEN.PK2-1C, by the LiAc/SS carrier DNA/PEG method. The wild-type strain used in this Example and the recombinant yeast strain (JHYS10) into which four genes (coex413-NpR4) were introduced are summarized in Table 6 below:

TABLE 6

| Strain | Genotype |
|---|---|
| CEN. PK2-1C | MATa ura3-52 trp1-289 leu2-3,112 his3 Δ1 |
| | MAL2-8^{C}SUC2 |
| WT-1 | CEN. PK2-1C harboring p413TEF |
| JHYS10 | CEN. PK2-1C harboring coex413-NpR4 |

The yeast strain (JHYS10) expressing WT-1 and coex412-NpR4 was pre-cultured in synthetic complex (SC)-His (including 20 g/L glucose) medium, and then inoculated at OD600=0.2 into 10 mL of the same medium, and cultured in a 100 mL flask under the conditions of 30° C. and 170 rpm for 48 hours. To quantify the concentration of mycosporine-like amino acids, specifically shinorine, two culture media were sampled at 2 mL each. One medium was dried in an oven and then the dry cell weight (DCW) was measured. Another medium was centrifuged to remove the supernatant, and then 1 mL of distilled water and 1.5 mL of chloroform were added to the yeast cells obtained, and then vortexed for 3 minutes to extract intracellular shinorine. After centrifugation, the aqueous layer was isolated and filtered, and used for measuring the intracellular shinorine concentration. An Ultimate3000 HPLC system (Thermo Fisher Scientific) equipped with an Agilent Eclipse XDB-C18 column (5 μm, 4.6×250 mm) was used, the column temperature was maintained at 40° C., and a solvent (water:acetonitrile=95:5) was flowed at a flow rate of 0.5 mL/min. Shinorine was detected with a UV-vis detector at 334 nm.

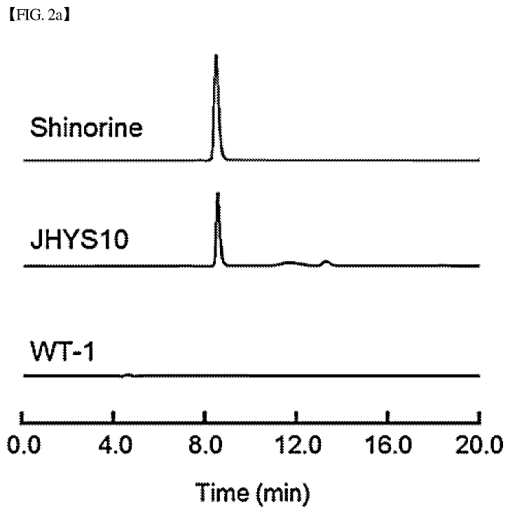

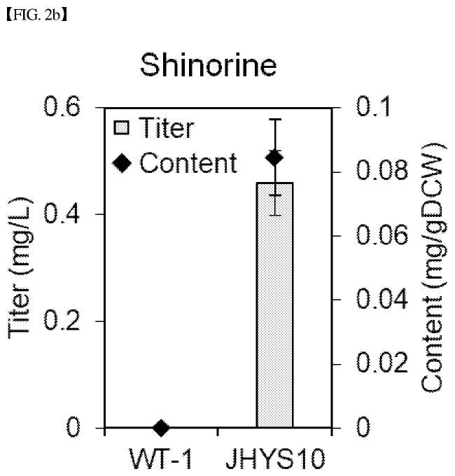

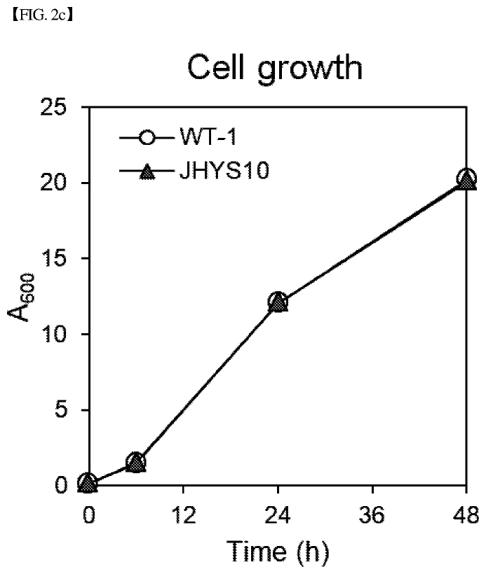

The results of the HPLC analysis as described above are shown in FIGS. 2a and 2b. As shown in FIGS. 2a and 2b, shinorine was not produced in WT-1 without introducing the shinorine biosynthetic gene, whereas a trace amount of shinorine was produced in JHYS10 (FIG. 2b: 0.46 mg/L and 0.085 mg/gDCW). Additionally, the OD600 value of the culture during the 48-hour incubation period was measured and shown in FIG. 2c. As shown in FIG. 2c, the two strains (WT-1 and JHYS10) did not show any difference in the cell growth rate.

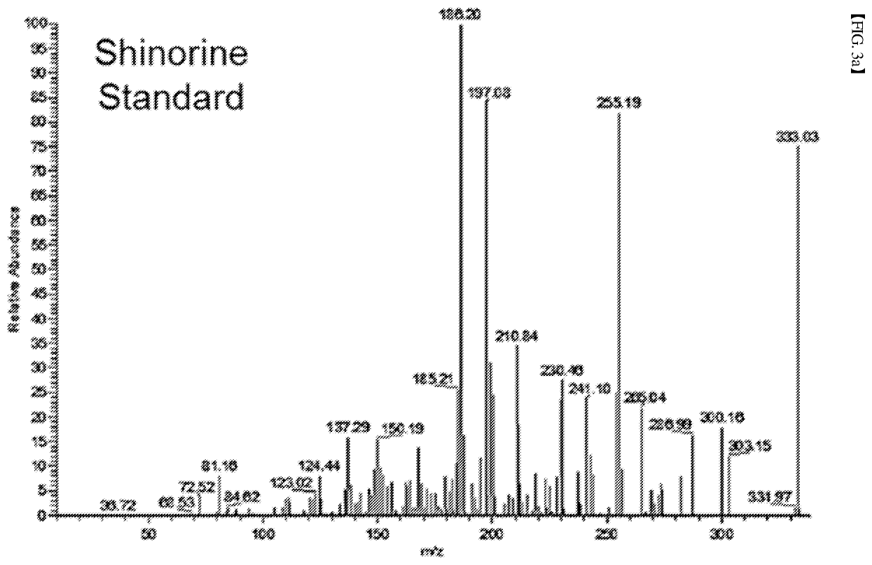

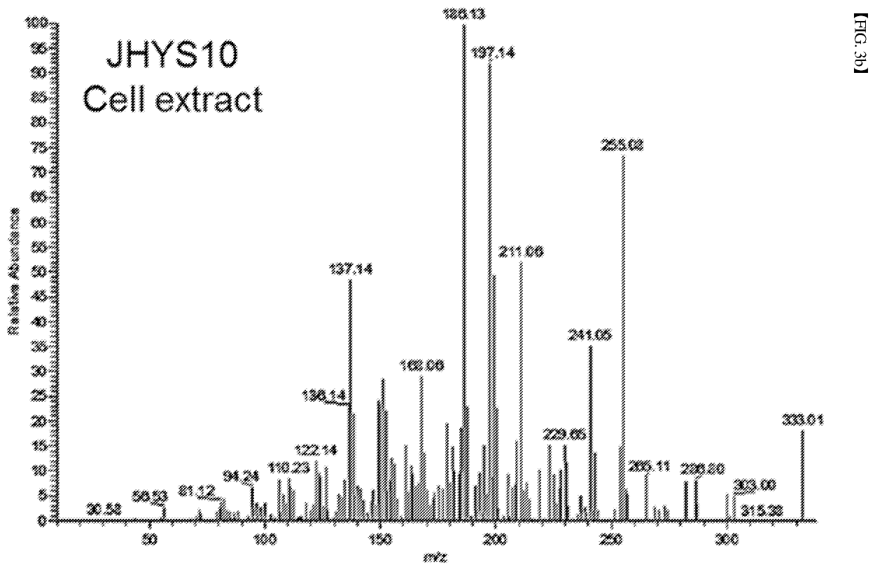

In order to confirm that the substance produced from the tested cell extract is shinorine, tandem mass spectrometry analysis (Thermofisher TSQ quantum access max) was performed. The obtained results are shown in FIGS. 3a and 3b. As shown in FIGS. 3a and 3b, it can be confirmed that the wild-type strain harboring p413GPD plasmid (WT-1) did not produce chinorine, whereas the yeast strain (JHYS10) into which the biosynthetic gene of N. punctiforme was introduced produced chinorine. These results show that the functional expression of the shinorine biosynthetic gene of N. punctiforme is possible in heterologous hosts.

### Example 3: Construction of a Vector for Delta-Integration for the Introduction of Mycosporine-Like Amino Acid Biosynthesis Gene into the Yeast Chromosome

Hundreds of retrotransposon sequences known as delta sequences are interspersed in the genome of S. cerevisae. When these delta sequences are targeted to the gene introduction site in the genome, a large number of genes can be randomly and simultaneously introduced into a chromosome. To introduce mycosporine-like amino acid biosynthesis gene into the delta site of the S. cerevisiae chromosome, an integration vector having a delta sequence at both ends was constructed, and the primers used here are summarized in Table 7 below:

TABLE 7

| | | Primer (F: | | SEQ |
|---|---|---|---|---|
| | | Forward, R: | | ID |
| PCR Poduct | Template used | Reverse) | Sequence(′→3′) | NO |
| Delta1 | Saccharomyces | Delta1 R | ATAGCGGCCGCATGTTTATATTC | 19 |
| | cerevisiae | | ATTGATCCTATTACA | |
| | genomic | Delta1 F | CACATTTCCCCGAAAAGTGCATT | 20 |
| | DNA (GenBank | | TAAATTGTTGGAATAGAAATCAA | |
| | Accession No. | | CTATC | |
| | JRIV01000000) | | | |
| Amp-Ori | p413GPD vector | Amp-Ori F | GCACTTTTCGGGGAAATGTG | 21 |
| | | Amp-Ori R | CTCAACATTCACCCATTTCTCAAT | 22 |
| | | | TTAAATCGCAGGAAAGAACATGT | |
| | | | GAG | |
| Delta2 | Saccharomyces | Delta2R | TGAGAAATGGGTGAATGTTGAG | 23 |
| | cerevisiae | Delta2F | GCGGCTAGCATAAAACGGAATGA | 24 |
| | genomic | | GGAATAATC | |
| | DNA (GenBank | | | |
| | Accession No. | | | |
| | JRIV01000000) | | | |
| P_{TEF1}-NpR5597- | coex413-NpR4 | Promoter up F | GCGGCTAGCGAGCTCGGAAACAG | 25 |
| T_{GPM1}-P_{TDH3}- | vector | | CTATGACCATGA | |
| NpR5600-T_{CYC1} | | Univ R | GACTACGCGTGCGGCCGCTAATG | 33 |
| | | | GCGCGCCATAGGGCGAATTGGGT | |
| | | | ACC | |
| P_{TEF1}-NpR5598- | coex413-NpR4 | Promoter up F | GCGGCTAGCGAGCTCGGAAACAG | 25 |
| T_{GPM1}-P_{TDH3}- | vector | | CTATGACCATGA | |
| NpR5599-T_{CYC1} | | Univ R | GACTACGCGTGCGGCCGCTAATG | 33 |
| | | | GCGCGCCATAGGGCGAATTGGGT | |
| | | | ACC | |

The obtained PCR product was used as a template, and [Amp-Ori-delta1-PTEF1-NpR5597-TGPM1-PTDH3-NpR5600-TCYC1-delta2] and [Amp-Ori-delta1-PTEF1-NPR5598-TGPM1-PTDH3-NpR5599-TCYC1-delta2] were constructed by an overlapping PCR method, and cloned using the NheI/NodI restriction enzyme site of the pUG6MCS vector (vector obtained by introducing cloning sites PacI, NheI, BamHI, SmaI, EcoRI, ApaI, KpnI, and AscI into pUG6(P30114)). These were named Delta6M-NPR1 and Delta6M-NPR2, respectively. The vectors used for the construction and the constructed vectors are summarized in Table 8 below:

TABLE 8

| Plasmid | Description |
|---|---|
| pUG6MCS | pUG6 plasmid containing additional restriction |
| | enzyme sites |
| Delta6M-NPR1 | Delta6M plasmid, P_{TEF1}-NpR5597-T_{GPM1}, |
| | P_{TDH3}-NpR5600-T_{CYC1} |
| Delta6M-NPR2 | Delta6M plasmid, P_{TEF1}-NpR5598-T_{GPM1}, |
| | P_{TDH3}-NpR5599-T_{CYC1} |

### Example 4: Delta-4-Integration of Mycosporine-Like Amino Acid Biosynthesis Gem

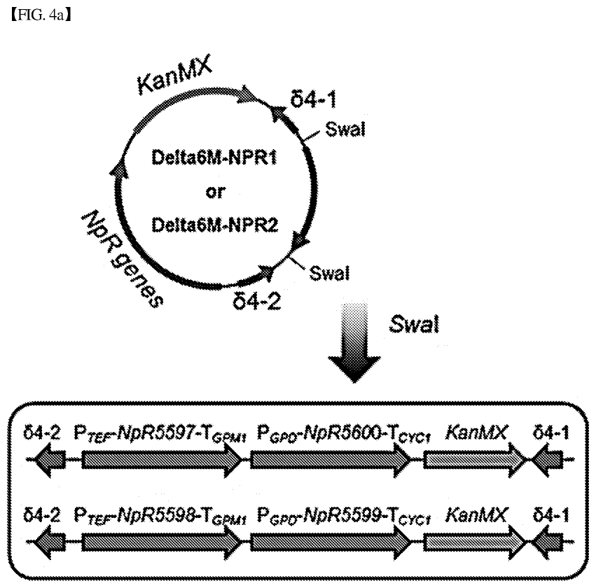

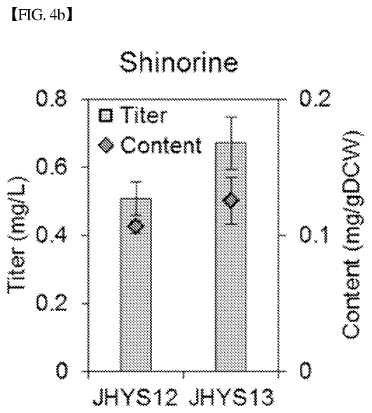

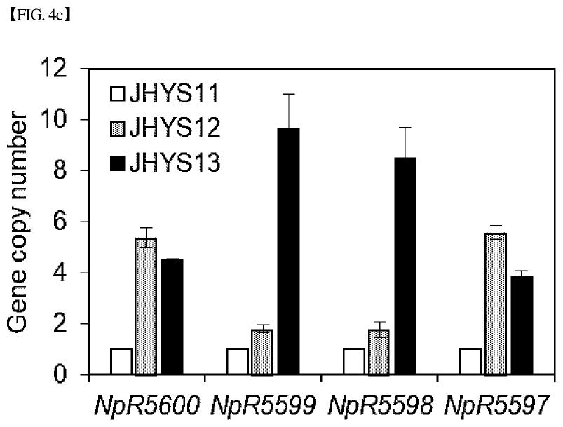

The Delta6M-NPR1 and Delta6M-NPR2 vectors obtained in Example 1.3 were constructed so that the delta1 and delta2 sequences were exposed at both ends when treated with the SwaI restriction enzyme. Therefore, the Delta6M-NPR1 and Delta6M-NPR2 vectors were treated with SwaI, and the two cassettes containing the NpR5597 and NpR5600 genes or the NpR5598 and NpR5599 genes were mixed, and co-transformed with S. cerevisiae CEN.PK2-1C (see FIG. 4a), and the obtained transformant was selected on YPD medium (10 g/L yeast extract, 20 g/L bacto-peptone, 20 g/L glucose) containing 2 mg/L of G418 (Geneticin).

A total of 18 transformants were selected. Among these, strains named JHYS12 and JHYS13 were pre-cultured in synthetic complex (SC) (containing 20 g/L glucose) medium, and then inoculated into the same medium of 10 mL at OD600=0.2, and incubated in a 100 mL flask under the conditions of 30° C. and 170 rpm for 48 hours. The amount of shinorine extracted from the cells of JHYS12 and JHYS13 was measured and shown in FIG. 4b. As shown in FIG. 4b, JHYS12 and JHYS13 produced higher amounts of shinorine than cells harboring coex413-NpR4 (JHYS10). More specifically, the shinorine production of the JHYS13 strain was 0.67 mg/L and 0.125 mg/gDCW, which were higher than that of the JHYS12 strain (0.51 mg/L and 0.106 mg/gDCW).

The copy numbers of the genes NpR5597, NpR5598, NpR5599, and NpR5600 introduced into the chromosomes of the strains named JHYS11. JHYS12, and JHYS13 among the selected transformants was confirmed by qPCR method. To determine the copy number of introduced genes, NpR5600, NpR5599, NpR5598, and NpR5597 were performed using gene-specific primers (see Table 9) and 1×SYBR Green I master mix (Roche Applied Science). qPCR was performed in 45 cycles (at 95° C. for 40 sec, at 60° C. for 20 see, at 72° C. for 20 sec) in LightCycler 480 II System (Roche Applied Science). The ACT1 gene was used as a control. Intersection (Cp) values were processed using LightCycler Software version 1.5 (Roche Applied Science), Expression levels were normalized to the target/reference ratio. Primers used for qPCR are shown in Table 9 below.

TABLE 9

| | Primer (F: Forward, | | SEQ |
|---|---|---|---|
| Target gene | R: Reverse) | Sequence (5′→3′) | ID NO |
| NpR5600 | NpR5600 qPCR F | ATGGCGAAGAGTTGCTTTCC | 40 |
| | NpR5600 qPCR R | GTGTGACCGTAGGCAATGAC | 41 |
| NpR5599 | NpR5599 qPCR F | CCCTCAGAATGGCAGAGGAA | 42 |
| | NpR5599 qPCR R | GACGCACTGTTTCACCATCA | 43 |
| NpR5598 | NpR5598 qPCR F | CTAGCTGCACCTTTGGAACC | 44 |
| | NpR5598 qPCR R | GAGCGAATCCCAGTCAATCG | 45 |
| NpR5597 | NpR5597 qPCR F | AAACTGACGATGGCGACTTG | 46 |
| | NpR5597 qPCR R | ACCAAGGTTGTCCCTTTGGA | 47 |

The copy number of genes measured as described above is shown in FIG. 4c. As shown in FIG. 4c, it was confirmed that JHYS12 genome (orange) contains 5 copies of NpR5600 and NpR5597, respectively, and contains 2 copies of NpR5599 and NpR5598, respectively, whereas JHYS13 (green) contains 4 copies of NpR5600 and NpR5597, respectively, and 9 and 8 copies of NpR5599 and NpR5598, respectively. In the meantime, it can be confirmed that strain JHYS11 contains only one of these four genes in its chromosome.

<Construction of a Yeast Strain that can Use Xylose as a Carbon Source and Production of Shinorine Utilizing the Same>

### Example 5: Construction of a Vector for Expressing Xylose Assimilation Enzyme

Since shinorine is produced from S7P, which is an intermediate material in the pentose phosphate pathway, the production of shinorine can be increased through the enhancement of the pentose phosphate pathway (see FIG. 1a). However, since yeast uses most of the glucose to produce pyruvate through glycolysis, it is difficult to enhance the pentose phosphate pathway when glucose is used as a carbon source. On the other hand, when xylose is used as a carbon source, xylulose-5-phosphate, which is a decomposition product of xylose, flows directly into the pentose phosphate pathway, thereby being able to enhance the pentose phosphate pathway.

Yeast does not naturally ferment xylose, but by introducing a heterologous pathway consisting of xylose isomerase (XI) or xylose reductase/xylitol dehydrogenase (XR/XDH), it can be grown using xylose. XI is the enzyme that consumes most of the xylose in bacteria, and has the advantage of being able to eliminate cofactor imbalance during xylose fermentation. However, since XI is decreased in enzymatic activity when expressed in yeast, the XR/XDH pathway is more often used for xylose fermentation in yeast. Xylose reductase (XR) and xylitol dehydrogenase (XDH) convert xylose to xylitol, which in turn converts xylitol to xylulose. Xylulose is converted to xylulose-5-phosphate (X5P) by xylulokinase (XK), and then, xylulose-5-phosphate is introduced into the pentose phosphate pathway (see FIG. 1a). Therefore, the use of xylose as a carbon source can increase S7P production through the pentose phosphate pathway. By introducing and expressing xylose assimilation genes encoding Pichia stipitis-derived XR (Xyl1), XDH (Xyl2) and XK (Xyl3) into yeast, a yeast strain capable of effectively fermenting xylose can be constructed.

To develop a xylose fermenting yeast strain, a plasmid capable of expressing XYL1 (encoding xylose reductase (XR)), XYL2 (encoding xylitol dehydrogenase (XDH)) and XYL3 (encoding xylulokinase (XK)) genes derived from Pichia stipitis under the control of the strong promoter PTDH3 were constructed. The primers used for the construction of the plasmid are shown in Table 10 below.

TABLE 10

| | | Primer (F: | | SEQ |
|---|---|---|---|---|
| PCR | | Forward, R: | | ID |
| Product | Template used | Reverse) | Sequence (5′→3′) | NO |
| XYL1 | pSR306-X123 | XYL1 F | GCGGGATCCATGCCTTCTATTAAGTTGA | 9 |
| | (Pichia stipitis- | | ACTC | |
| | derived XYL1, | XYL1 R | GCGCTCGAGTTAGACGAAGATAGGAAT | 10 |
| | XYL2, XYL3 | | CTTGT | |
| XYL2 | expression plasmid) | XYL2 F | GCGGGATCCATGACTGCTAACCCTTCCT | 11 |
| | | | T | |
| | | XYL2 R | GCGCTCGAGTTACTCAGGGCCGTCAAT | 12 |
| | | | G | |
| XYL3 | | XYL3 F | GCGGGATCCATGACCACTACCCCATTTG | 13 |
| | | XYL3 R | GCGCTCGAGTTAGTGTTTCAATTCACTT | 14 |
| | | | TCCATC | |
| P_{TDH3}- | p416GPD-XYL2 | UnivF2 | GACTCGCGCGCGGGAACAAAAGCTGGA | 32 |
| XYL2- | | | GCTC | |
| T_{CYC1} | | UnivR | GACTACGCGTGCGGCCGCTAATGGCGC | 33 |
| | | | GCCATAGGGCGAATTGGGTACC | |
| PTDH3- | p416GPD-XYL3 | UnivF2 | GACTCGCGCGCGGGAACAAAAGCTGGA | 32 |
| XYL3- | | | GCTC | |
| T_{CYC1} | | UnivR | GACTACGCGTGCGGCCGCTAATGGCGC | 33 |
| | | | GCCATAGGGCGAATTGGGTACC | |

pSR-306-X123 which is Pichia stipites-derived XYL1, XYL2, and XYL3 expression plasmid (Pichia stipites-derived XYL1, XYL2, and XYL3 gene expression cassettes TDH3p-XYL1-TDH3t, PGK1p-XYL2-PGK1t, and TDH3p-XYL3-TDH3p-cloned pSR306 vector) was subjected to PCR using the primers in Table 7, and the XYL1, XYL2, and XYL3 genes were respectively secured from pSR306-X123, treated with BamHI/XhoI restriction enzymes, and cloned into the p16GPD plasmid (ATCC® 87360™) to construct p416GPD-XYL1, p416GPD-XYL2, and p416GPD-XYL3 plasmids. The constructed p16GPD-XYL2 and p416GPD-XYL3 were used as a prototype, and a DNA fragment containing a promoter and a terminator was secured by PCR using the primer pair of SEQ ID NOs: 32 and 33 (see Table 7), treated with MauBI/NodI restriction enzyme, cloned one after another into the p416GPD-XYL1 vector, and finally, a coex416-XYL vector containing all of the XYL1, XYL2, and XYL3 genes was constructed.

### Example 6: Evaluation of Xylose-Consuming Ability and Mycosporine-Like Amino Acid-Producing Ability of Yeast Introduced with Xylose Assimilation Enzyme

The coex416-XYL constructed in Example 5 was transformed into the shinorine-producing strain JHYS13 obtained in Example 4 by the LiAc/SS carrier DNA/PEG method. The recombinant yeast (S. cerevisiae) strains used in this Example are summarized in Table 11 below.

TABLE 11

| Strain | Genotype |
|---|---|
| JHYS13 | Selected strain from delta-integration of |
| | P_{TEF1}-NpR5597-T_{GPM1}-P_{TDH3}-NpR5600-T_{CYC1} and |
| | P_{TEF1}-NpR5598-T_{GPM1}-P_{TDH3}-NpR5599-T_{CYC1} into |
| | CEN. PK2-1C |
| JHYS13-1 | JHYS13 harboring p416GPD |
| JHYS13-2 | JHYS13 harboring coex416-XYL |

The JHYS13 strain transformed with an empty vector p416GPD (JHYS13-1; control) and the JHYS13 strain (JHYS13-2) transformed with coex416-XYL were cultured in various media including xylose and glucose, and the xylose-consuming ability was confirmed. JHYS13-1 and JHYS13-2 strains were pre-cultured into SC-Ura (containing 20 g/L glucose) medium, and then inoculated at OD600=0.2 in 10 mL of SC-Ura (with 20 g/L glucose), SC-Ura (with 10 g/L glucose and 10 g/L xylose), SC-Ura (with 2 g/L glucose and 18 g/L xylose), and SC-Ura (with 20 g/L xylose) medium in a 100 mL flask, and cultured under the conditions of 30° C. and 170 rpm.

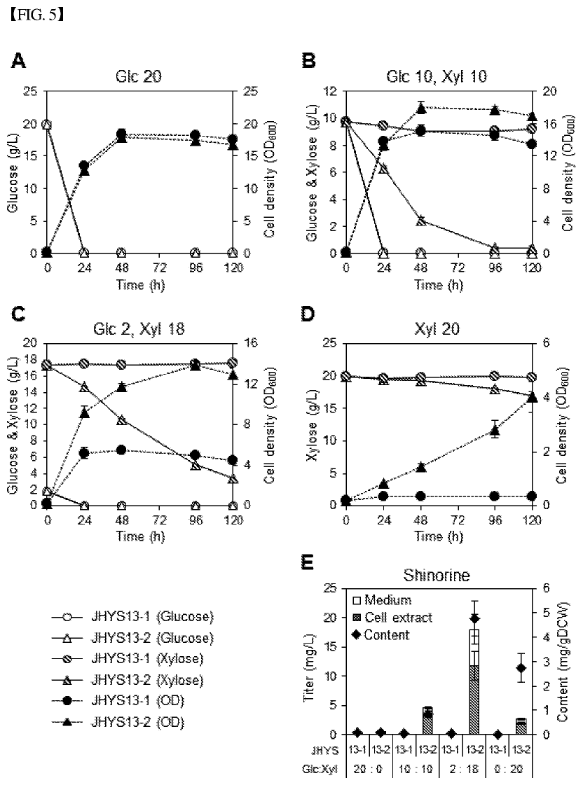

JHYS13-1 and JHYS13-2 strains were cultured in the above four types of media for 120 hours. To measure the concentration of glucose and xylose in the medium, 500 μl of the culture supernatant was collected, filtered through a 0.22-μm syringe filter, and analyzed using HPLC. 5 mM H₂SO₄ was flowed as a solvent into an UltiMate 3000 HPLC system (Thermo Fisher Scientific) equipped with an Aminex HPX-87H column (300 mm×7.8 mm, 5 μm, Bio-Rad) at 60° C. at a flow rate of 0.6 mL/min, and concentrations of glucose and xylose were measured through a refractive index (RI) detector (35° C.). Cell density (OD600 value) of the culture, and shinorine production (titer (mg/L) and content (mg/gDCW) in the cell extract and medium) during culture for 120 hours were measured, and the results are shown in FIG. 5. To quantify the concentration of shinorine, two culture media were sampled at 2 mL each. One medium was dried in an oven and then the dry cell weight (DCW) was measured. Another medium was centrifuged, and then the supernatant was filtered, and the concentration of shinorine secreted into the medium was measured by HPLC. Yeast cells separated from the supernatant were vortexed for 3 minutes after adding 1 mL of distilled water and 1.5 mL of chloroform, and the intercellular shinorine was extracted. After centrifugation, the aqueous layer was isolated and filtered, and used and used for measuring the intracellular shinorine concentration. An Ultimate3000 HPLC system (Thermo Fisher Scientific) equipped with an Agilent Eclipse XDB-C18 column (5 μm, 4.6×250 mm) was used, the column temperature was maintained at 40° C., and a solvent (water:acetonitrile=95:5) was flowed at a flow rate of 0.5 mL/min. Shinorine was detected with a UV-vis detector at 334 nm.

As shown in FIG. 5, it was confirmed that JHYS13-1 and JHYS13-2 strains showed similar growth rates in a medium containing only glucose (20 g/L), and the production of shinorine was also similar (see A and E of FIG. 5). Additionally, it was confirmed that the JHYS13-1 strain could not consume xylose (see the yellow circle graphs in B, C and D of FIG. 5). On the other hand, it was confirmed that the JHYS13-2 strain could consume xylose (see the yellow triangle graphs in B, C, and D of FIG. 5), and it was confirmed that the higher the consumption of xylose, the higher the production of shinorine (comparison of the yellow triangle graphs of B, C, and D of FIG. 5 with the results of E of FIG. 5). In particular, in the medium containing only xylose (see D of FIG. 5), the consumption rate of xylose was very slow, and in the medium containing a small amount of glucose, the tendency to consume xylose was better (see B and C of FIG. 5). These results show that the use of xylose through the pentose phosphate pathway is effective in increasing the production of shinorine, but the efficiency of using xylose is low, whereby when a certain amount of glucose is contained in the medium, it is more advantageous for promoting cell growth and shinorine production.

### Example 7: Construction of NTS-Integration Vector for Introducing Xylose Anabolic Enzyme into Yeast Chromosome

After confirming the effect of producing shinorine using xylose as a substrate in the same manner as in Example 6, a fermented xylose strain was developed by introducing XYL1, XYL2, and XYL3 genes into chromosomes. The ribosomal DNA (rDNA) region of the XII chromosome in yeast (S. cerevisiae) repeatedly contains 150 impermeable spacers (NTS1 and NTS2) with a size of 9.1 kb. When this sequence is used as a gene introduction sequence, multiple copies of genes can be randomly introduced into a chromosome at the same time. Vectors for introducing XYL1, XYL2, XYL3 (XYL) genes into the NTS site were constructed as follows.

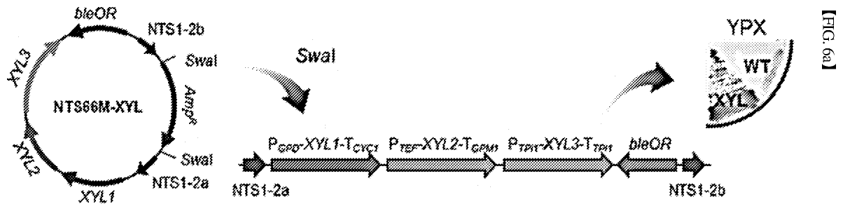

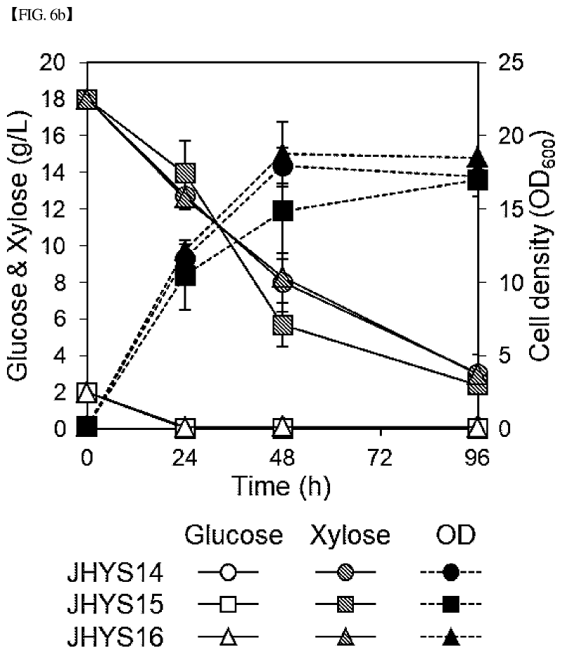

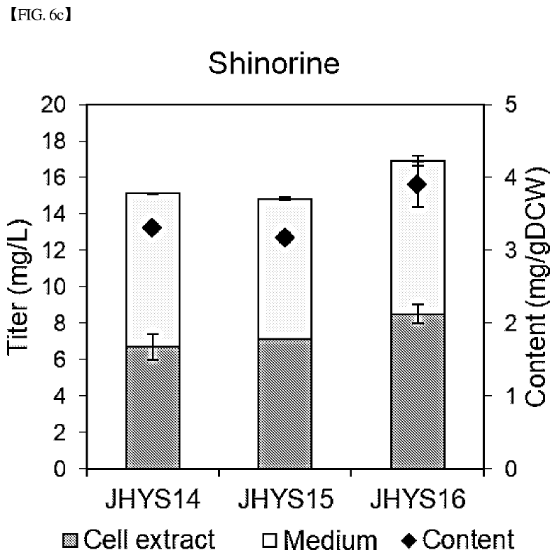

DNA fragments of NTS1-2a (400 bp) and NTS1-2b (400 bp) were amplified from the yeast genome by PCR with the primers in Table 12 below. The AmpR cassette and the bleOR DNA cassette were secured from the pUG66 vector by PCR using the primers in Table 12 below. Overlapping PCR was performed on the four PCR products thus obtained to construct one linked fragment NTS1-2a-bleOR-AmpR-NTS1-2b. The resulting DNA fragment NTS1-2a-bleOR-AmpR-NTS1-2b was ligated to the XYL1 expression cassette (PTDH3-XYL1-TCYC1) via NheI and Nod sites to construct an NTS66M-XYL1 plasmid. XYL2 was cloned into p414TEF (ATCC® 87364™) using BamHI and XhoI restriction enzymes to construct a p414TEF-XYL2 vector. Additionally, XYL3 was cloned into coex414TPI1 vector (see Korean Unexamined Patent Publication No. 10-2016-0093492) using BamHI and XhoI restriction enzymes to construct p414TPI1-XYL3. This was used as a template, and XYL2 expression cassette (PTEF1-XYL2-TGPM1) and XYL3 expression cassette (PTPI1-XYL3-TTPI1) with MauBI and Nod sites were secured by PCR, and sequentially cloned between the AscI and Nod sites of NTS66M-XYL1, and an NTS66M-XYL plasmid for introducing the NTS site of the XYL1, XYL2, XYL3 (XYL) genes was constructed (see FIG. 6a). The primers used in the PCR are summarized in Table 12 below.

TABLE 12

| | | Primer (F: | | SEQ |
|---|---|---|---|---|
| PCR | | Forward, R: | | ID |
| Product | Template used | Reverse) | Sequence (5′→3′) | NO |
| NTS1-2a | Saccharomyces | NTS1-2aF | CCGAGCGTGAAAGGATTTGCC | 30 |
| | cerevisiae | NTS1-2aR | GCGGCTAGCCAACCATTCCATATCTGT | 31 |
| | genomic | | TAAG | |
| NTS1-2b | DNA (GenBank | NTS1-2bF | GCGAAGTAAATTTTTGGCG | 28 |
| | Accession No. | NTS1-2b AmpR | TTTCCCCGAAAAGTGCATTTAAATCTA | |
| | JRIV01000000) | | GTTTCTTGGCTTCCTATG | |
| AmpR | pUG66 | Amp-QriF | GCACTTTTCGGGGAAATGTG | 21 |
| | (EUROSCARF) | Amp-OriR | CTCAACATTCACCCATTTCTCAATTTA | 22 |
| | | | AATCGCAGGAAAGAACATGTGAG | |
| bleOR | pUG66 (P30116) | TEF prom F | GCCAAAAATTTACTTCGCGCGGCCCGA | 27 |
| | | | CATGGAGGCCCAGAAT | |
| | | NTS term R | GCGGGCGCGCCGCGGCCGCTAAGGGT | 26 |
| | | | TCTCGAGAGCTC | |
| P_{TEF1}- | p414TEF-XYL2 | UnivF2 | GACTCGCGCGCGGGAACAAAAGCTGG | 32 |
| XYL2- | | | AGCTC | |
| T_{GPM1} | | UnivR | GACTACGCGTGCGGCCGCTAATGGCG | 33 |
| | | | CGCCATAGGGCGAATTGGGTACC | |
| P_{TPI1}- | p414TPI1-XYL3 | UnivF2 | GACTCGCGCGCGGGAACAAAAGCTGG | 32 |
| XYL3- | | | AGCTC | |
| T_{TPI1} | | UnivR | GACTACGCGTGCGGCCGCTAATGGCG | 33 |
| | | | CGCCATAGGGCGAATTGGGTACC | |

### Example 8: NTS-Integration of Xylose Assimilation Enzyme and Evaluation of Xylose-Consuming Ability and Mycosporine-Like Amino Add-Producing Ability of Yeast Strains Produced Therefrom

The NTS66M-XYL plasmid prepared in Example 7 was treated with a SwaI restriction enzyme, so that the NITS site was exposed at both ends of the DNA (see FIG. 6a). To insert the XYL gen into the rDNA region of the yeast chromosome, the prepared NTS66M-XYL plasmid was cleaved with SwaI, and transformed into JHYS13 strain by LiAc/SS carrier DNA/PEG method, and then selected on YPX (10 g/L yeast extract, 20 g/L bacto peptone and 20 g/L xylose) plates containing 500 mg/L zeocin. The recombinant yeast strains selected in this way are summarized in Table 13 below.

TABLE 13

| Strain | Genotype |
|---|---|
| JHYS14 | Selected strain from NTS-integration of |
| | P_{TDH3}-XYL1-T_{CYC1}-P_{TEF1}-XYL2-T_{GPM1}-P_{TPI1}-XYL3-T_{TPI1} |
| | into CEN. PK2-1C |
| JHYS15 | Selected strain from NTS-integration of |
| | P_{TDH3}-XYL1-T_{CYC1}-P_{TEF1}-XYL2-T_{GPM1}-P_{TPI1}-XYL3-T_{TPI1} |
| | into CEN. PK2-1C |
| JHYS16 | Selected strain from NTS-integration of |
| | P_{TDH3}-XYL1-T_{CYC1}-P_{TEF1}-XYL2-T_{GPM1}-P_{TPI1}-XYL3-T_{TPI1} |
| | into CEN. PK2-1C |

The selected strains was inoculated into SC medium (2 g/L glucose, 18 g/L xylose) at an OD600=0.2, and cultured at 30° C. and 170 rpm for 96 hours. The concentration of glucose and xylose in the medium and the cell density (OD600 value) of the culture were measured and shown in FIG. 6b, and the shinorine production (titer (mg/L) and content (mg/gDCW) in the cell extract and medium) during the 96-hour culture was measured and shown in FIG. 6c.

As shown in FIGS. 6b and 6c, all three strains selected above have xylose-consuming ability and shinorine-producing ability. In particular, the JHYS16 strain showed the highest shinorine production (17.72 mg/L and 8.7 mg/gDCW).

The JHYS16 strain was deposited with the Korean Culture Center of Microorganisms (KCCM) located in Seodaemun-gu, Seoul, Korea on Nov. 14, 2019 under the Budapest Treaty, and was given an accession number KCCM12628P.

<Production of Mycosporine-Like Amino Acids Through the Enhancement of the Pentose Phosphate Pathway>

### Example 9: Construction of Transaldolase (Tal1) Deletion Strain

To further improve the production of shinorine in the JHYS16 strain, which was confirmed to have excellent shinorine-producing ability in Example 8, a plan was devised to increase the production of S7P, which is an intermediate product. For this purpose, it was attempted to remove TAL1, which is a transaldolase involved in the conversion reaction between S7P and glyceraldehyde 3-phosphate in the pentose phosphate pathway (see FIG. 1a). The CRISPR-Cas9 system was used to remove TAL1 using the CRISPR-Cas9 system. Specifically, the coding gene and gRNA fragment (PSNR52-structural component-TSUP4) of Cas9 (NP_269215.1) of Streptococcus pyogenes were cloned into p413TEF (ATCC® 87362™) to produce a coex413-Cas9-gRNA plasmid. TAL1 target gRNA (sgRNA) was designed by Yeastriction v0.1 (http://yeastriction.tnw.tudelft.nl), and inserted into coex413-Cas9-gRNA by DpnI-mediated site-directed mutagenesis based on PCR to construct coex413-Cas9-TAL1gRNA. The primers and the prepared plasmids used in the PCR are shown in Tables 14 and 15 below, respectively.

TABLE 14

| | | Primer (F: | | SEQ |
|---|---|---|---|---|
| PCR | | Forward, R: | | ID |
| Product | Template used | Reverse) | Sequence (5′→3′) | NO |
| TAL1 | — | TAL1_delF | TAGTACGATAGTAAAATACTTCTCGAACTC | 34 |
| deletion | | | GTCACATATACGTGTACATAGGAAGTATCT | |
| cassette | | TAL1_delR | AGAAACGTGCATAAGGACATGGCCTAAAT | 35 |
| | | | TAATATTTCCGAGATACTTCCTATGTACAC | |
| | | | G | |
| coex413- | coex413- | TAL1 gRNA F | AACTAACCCATCATTGATCTGTTTTAGAGC | 38 |
| Cas9- | Cas9-gRNA | | TAGAAATAGC | |
| TAL1gRNA | | TAL1 gRNA R | AGATCAATGATGGGTTAGTTGATCATTTAT | 39 |
| | | | CTTTCACTGC | |

TABLE 15

| Plasmid | Description |
|---|---|
| coex413-Cas9-TAL1gRNA | CEN/ARS plasmid, HIS3, |
| | P_{TDH3}-CAS9-T_{TPI1}, |
| | P_{SNR52}-TAL1gRNA-T_{SUP4} |

Coex413-Cas9-TAL1gRNA expressing guide RNA (gRNA) targeting the Cas9 gene and TAL1 gene and TAL1 deletion cassette having homologous arms above and below the TAL1 ORF were transformed into JHYS16 cells, and then SC-His (2 wt % glucose), a TAL1-deletion strain was selected. TAL1 deletion was confirmed by PCR (primers used: TAL1_CF primer: CGGGAATAAAAGCGGAACT (SEQ ID NO: 36); TAL1_CR primer: GGTGGITCCGGATGTIT (SEQ ID NO: 37)). After confirming the TAL1 deletion by PCR, it was cultured overnight in YPD (10 g/L yeast extract, 20 g/L bactopeptone and 20 g/L glucose) medium, and the coex413-Cas9-TAL1gRNA plasmid was removed. The Tal1-deletion strain thus obtained was named JHYS17.

### Example 10: Evaluation of Mycosporine-Like Amino Add-Producing Ability of Transaldolase (Tal1) Deletion Strain

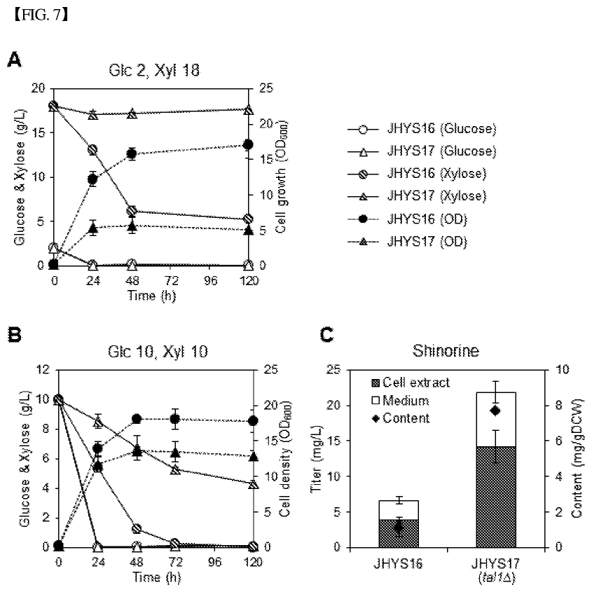

The prepared JHYS16 and JHYS17 were inoculated into SC medium (containing 2 g/L glucose and 18 g/L xylose) or SC medium (containing 10 g/L glucose and 10 g/L xylose) at an OD600=0.2, respectively, and cultured under the conditions of 30° C. and 170 rpm for 120 hours. The glucose and xylose concentrations in the medium, the cell density of the culture (OD600 value), and the shinorine production (titer (mg/L) and content (mg/gDCW) in the cell extract and the medium) during the 120 hour-culture were measured and shown in FIG. 7.

The JHYS17 strain obtained by deleting TAL1 with JHYS16 was cultured in SC medium (containing 2 g/L glucose and 18 g/L xylose). As a result, it was confirmed that severe growth defects were seen in the xylose-rich medium (A of FIG. 7), confirming that TAL1 is essential for xylose assimilation.

Therefore, the ratio of glucose to xylose was changed to alleviate the growth defects and the culture was performed. JHYS16 and JHYS1 were cultured in SC medium (containing 10 g/L glucose and 10 g/L xylose), and the results are shown in B of FIG. 7. As shown in B of FIG. 7, JHYS16 and JHYS17 showed similar glucose consumption rates, but unlike JHYS16, which consumed most of the medium xylose, JHYS17, which was defective in xylose anabolic activity, consumed only 5.7 g/L xylose. However, JHYS17 produced about 3.3 times higher titer (21.9 mg/L) and 7.2 times higher amount (7.67 mg/gDCW) of shinorine than JHYS16 (see C of FIG. 6). The shinorine production level of JHYS17 was much higher than that produced in JHYS16 cultured in a medium containing 2 g/L glucose and 18 g/L xylose (see C of FIG. 5). Therefore, when TAL1 was deleted, it was confirmed that S7P generated from xylose may be more useful for shinorine biosynthesis, but xylose assimilation action was limited due to the incomplete pentose phosphate pathway.

### Example 11: Construction of a Vector for Overexpression of Transcription Regulator Stb5 and Transketolase (Tkl1) in Yeast

Since JHY17 is defective in the pentose phosphate pathway, enhancing the expression of other genes involved in the pentose phosphate pathway can aid in xylose fermentation and subsequent shinorine production. Therefore, as overexpression target genes, Stb5 (GenBank accession no. JRIV01000036.1) encoding a transcriptional regulatory factor and TKL1 (GenBank accession no. JRIV01000030.1) encoding a transketolase were selected. Stb5 activates genes in the pentose phosphate pathway, including glucose-6-phosphate dehydrogenase (ZWF1), 6-phosphogluconate dehydrogenase (GND1), TKL1 and other NADPH production pathways to produce NADPH, which plays an important role in the oxidative stress response. Tkl1 forms a reversible link between the pentose phosphate pathway and glycolysis which are two major metabolic pathways (see FIG. 1a), which plays an important role when culturing yeast using xylose as a carbon source. To confirm the overexpression effect of STB5 and TKL1, plasmids expressing these genes individually or in combination were constructed. Fragments of each gene were secured by PCR, and the primers used for PCR are shown in Table 16 below.

TABLE 16

| | | Primer (F: | | |
|---|---|---|---|---|
| PCR | | Forward, | | SEQ ID |
| Product | Template used | R: Reverse) | Sequence (5′→3′) | NO |
| TKL1 | Saccharomyces | TKL1 F | GCGGGATCCATGACTCAATTCA | 117 |
| | cerevisiae | | CTGACA | |
| | genomic | TKL1 R | GCGCTCGAGTTAGAAAGCTTTT | 118 |
| | DNA (GenBank | | TTCAAAGGAG | |
| | Accession No. | STB5F | GCGGGATCCATGGATGGTCCCA | 115 |
| STB5 | JRIV01000000) | | ATTTTG | |
| | | STB5R | GCGGTCGACTCATACAAGTTTA | 116 |
| | | | TCAACCCAAG | |
| P_{TDH3}- | p414GPD- | Univ F2 | GACTCGCGCGCGGGAACAAAA | 332 |
| TKL1-T_{CYC1} | TKL1 | | GCTGGAGCTC | |
| | | Univ R | GACTACGCGTGCGGCCGCTAAT | 333 |
| | | | GGCGCGCCATAGGGCGAATTGG | |
| | | | GTACC | |

TKL1 was cloned into p414GPD vector (ATCC®87356™) using BamHI and XhoI as restriction enzyme sites so that is was expressed under the control of PTDH3, thereby obtaining p414GPD-TKL1 (Table 16). For overexpression using a strong promoter, STB5 was cloned into the p414ADH vector (ATCC®87372™) to be expressed under the control of the weak promoter PADH1 due to its growth inhibitory effect on yeast strains. STB5 DNA fragment was treated with BamHI, SalI, and p414ADH was treated with BamHI and XhoI, and cloned (p414ADH-STB5). The TKL1 expression cassette (PTDH3-TKL1-TCYC1) having MauBI and Nod sites obtained by PCR using p414GPD-TKL1 as a template was cloned between the Ascl and NotI sites of p414ADH-STB5 to construct a coex414-STB5-TKL1 plasmid. The constructed plasmids are shown in Table 17 below.

TABLE 17

| Plasmid | Description |
|---|---|
| p414ADH-STB5 | CEN/ARS plasmid, TRP1, P_{ADH1}-STB5-T_{CYC1} |
| p414GPD-TKL1 | CEN/ARS plasmid, TRP1, P_{TDH3}-TKL1-T_{CYC1} |
| coex414-STB5-TKL1 | CEN/ARS plasmid, TRP1, P_{ADH1}-STB5-T_{CYC1}, |
| | P_{TDH3}-TKL1-T_{CYC1} |

### Example 12: Evaluation of Mycosporine-Like Amino Add-Producing Ability Through Overexpression in Yeast of Transcription Regulators Stb5 and Tkl1 of the Pentose Phosphate Pathway

To determine whether the enhancement of the pentose phosphate pathway via additional gene overexpression is effective in increasing shinorine production, JHYS17 strain was transformed with p414ADH-STB5, p414GPD-TKL1, or coex414-STB5-TKL1 plasmids using the LiAc/SS carrier DNA/PEG method. The transformed yeast strains are shown in Table 18 below.

TABLE 18

| Strain | Genotype |
|---|---|
| JHYS17 | JHYS16 tal1Δ |
| JHYS17-1 | JHYS17 harboring p414GPD |
| JHYS17-2 | JHYS17 harboring p414ADH-STB5 |
| JHYS17-3 | JHYS17 harboring p414GPD-TKL1 |
| JHYS17-4 | JHYS17 harboring coex414GPD-STB5-TKL1 |

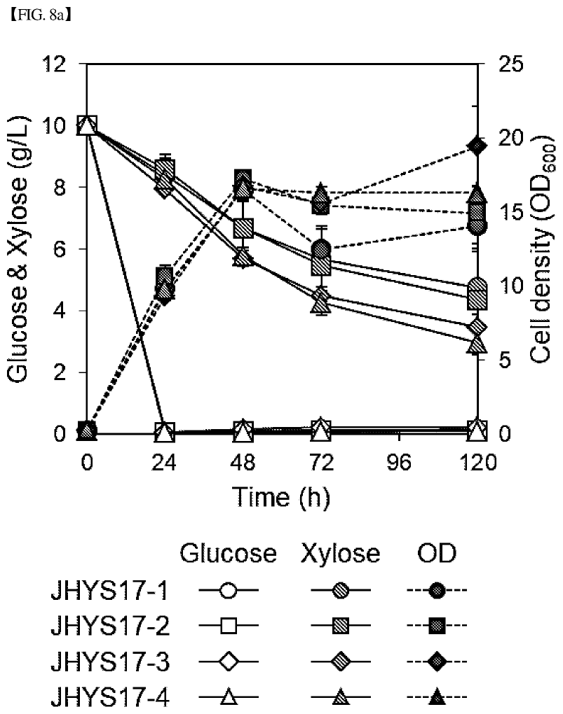

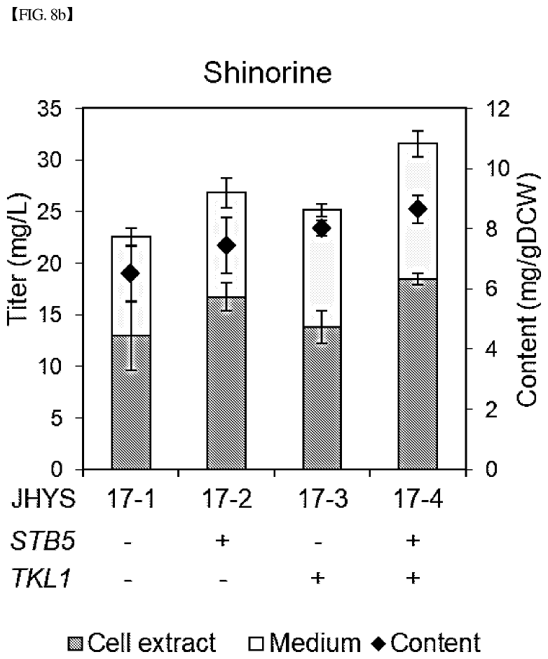

Each of the obtained transformed yeast stains was inoculated into SC-Trp (10 g/L glucose, 10 g/L xylose) medium at OD600=0.2. While culturing under the conditions of 30° C. and 170 rpm for 120 hours, the glucose and xylose concentrations in the medium and the cell density (OD600 value) of the culture were measured and shown in FIG. 8a, and the shinorine production (titer (mg/L) and content (mg/gDCW) in the cell extract and medium) during the 120-hour culture was measured and shown in FIG. 8b.

As shown in FIG. 8b, when both genes (STB5 and TKL1) were overexpressed, it was effective in increasing shinorine production. JHYS17-2 and JHYS17-3 strains overexpressing STB5 or TKL1 consumed a small amount of xylose as compared with the control JHYS17-1 strain harboring the empty vector, and the shinorine production was also increased (FIG. 8b). The JHYS17-4 strain overexpressing both STB5 and TKL1 had the highest shinorine production, and the consumption of xylose was also high. Since overexpression of these genes was effective in increasing the rate of xylose consumption (FIG. 8a), overexpression of these genes can alleviate the problem of cell growth degradation that occurs during xylose assimilation in the Tal-deficient JHYS17 strain.

### Example 13: Search for the Optimal Ratio of Glucose and Xylose in the Medium

Due to the inefficient utilization of xylose in Tal1-deficient strains, further addition of glucose to the medium can promote cell growth. On the other hand, the important intermediate S7P for shinorine production is provided mainly through xylose assimilation. Therefore, the ratio of glucose to xylose in the medium can be adjusted to obtain appropriate cell growth and maximum shinorine production effect.

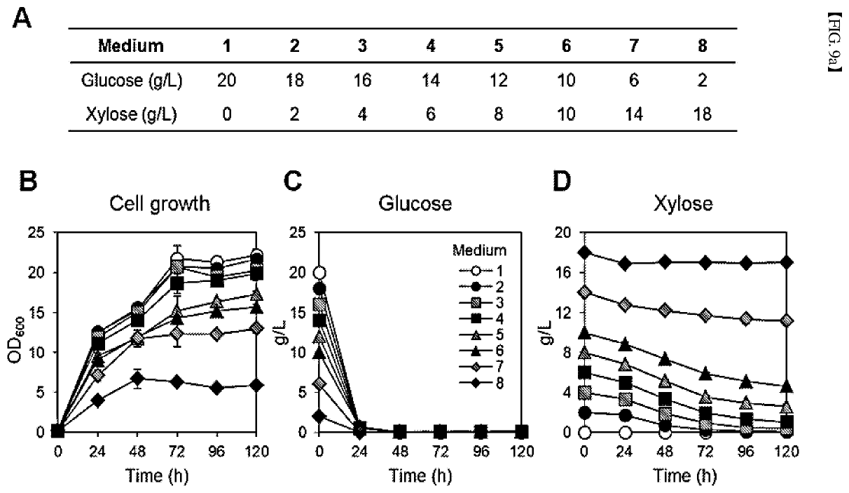

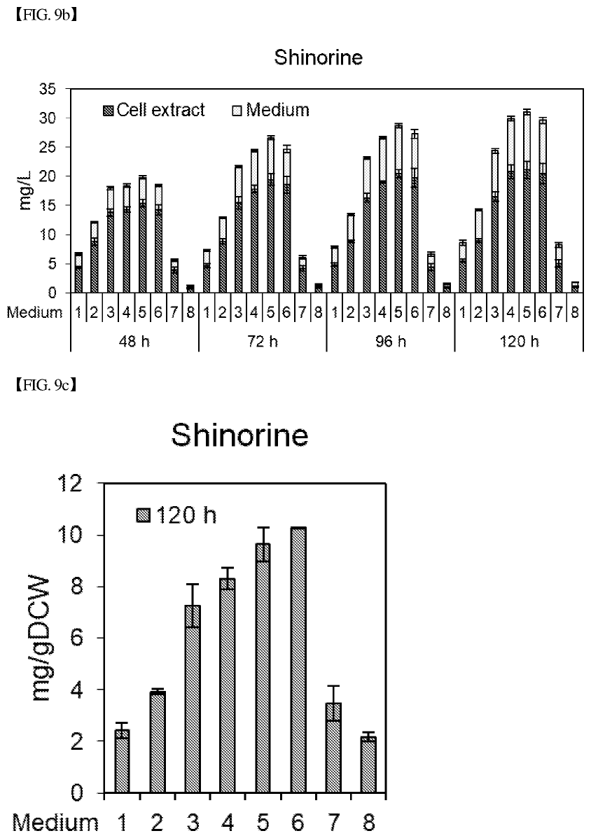

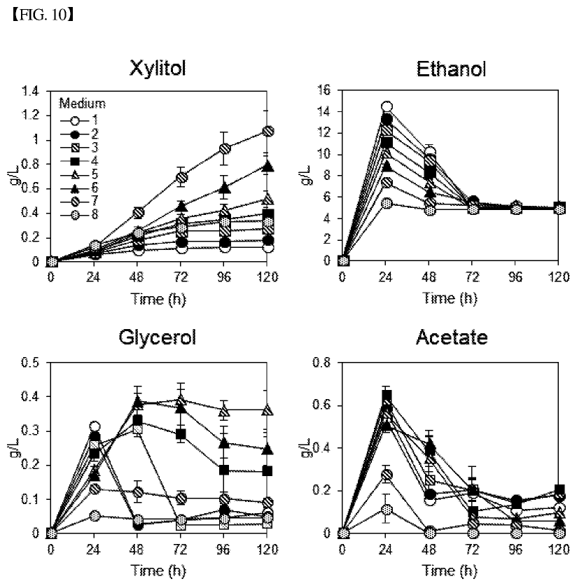

To find the optimal ratio of these carbon sources (glucose and xylose), eight different media containing xylose and glucose in various proportions (see A of FIG. 9a) were inoculated with the JHYS17-4 strain at OD600=0.2 while maintaining the total carbon source concentration at 20 g/L, and cultured at 30° C. and 170 rpm for 120 hours. The cell density (OD600 value) of the culture obtained by the above culture and the concentration of glucose and xylose in the medium were measured and shown in B, C and D of FIG. 9a, respectively, and the shinorine production (titer (mg/L) and content (mg/gDCW) in cell extract and medium) during the 120-hour culture were measured and shown in FIGS. 9b and 9c.

As a result, as the xylose ratio of the medium increased (i.e., as the glucose ratio decreased), the cell growth rate decreased (B of FIG. 9a). However, as the xylose concentration of the medium increased to 8 g/L, shinorine production increased. When the strain was cultured in medium #5 containing 12 g/L of glucose and 8 g/L of xylose, 12 g/L of glucose and 5.4 g/L of xylose were consumed (C and D in FIG. 9a), and the most shinorine was produced (31.0 mg/L (FIG. 9b) and 9.6 mg/gDCW (FIG. 9c)). Also, the shinorine content was highest when cultured in medium #6 containing 10 g/L glucose and 10 g/L xylose (10.27 mg/gDCW (FIG. 9c)). When the xylose concentration in the medium was higher than 14 g/L, cell growth was inhibited (A of FIG. 9), and the reduced cell growth had a negative effect on shinorine production (FIGS. 9b and 9c).

Additionally, the content of by-products (xylitol, ethanol, glycerol, and acetate) produced during the culture was measured and shown in FIG. 10. To determine the concentration of xylitol, ethanol, glycerol and acetate in the medium, 500 μl of the culture supernatant was collected and filtered with 0.22-μm syringe filter and then analysis was performed using HPLC. 5 mM H₂SO₄ was flowed as a solvent into an UltiMate 3000 HPLC system (Thermo Fisher Scientific) equipped with an Aminex HPX-87H column (300 mm×7.8 mm, 5 μm, Bio-Rad) at 60° C. at a flow rate of 0.6 mL/min, and the concentration of by-products was measured through a refractive index (RI) detector (35° C.). As can be seen in FIG. 10, ethanol was the main by-product in most media conditions, but xylitol was accumulated as a by-product of xylose assimilation. The accumulation of xylitol can be an important factor that slows the consumption rate of xylose.

As described above, respective descriptions and embodiments disclosed in the present disclosure can also be applied to other descriptions and embodiments. That is, all combinations of various elements disclosed in the present disclosure fall within the scope of the present disclosure. In addition, the scope of the present disclosure is not limited by the specific description below. In addition, one of ordinary skill in the art can recognize or identify a number of equivalents with regard to certain aspects of the present disclosure only by routine experimentation. Further, such equivalents are intended to be included in the present disclosure.

### Accession Number

Name of depositary institution: Korea Microorganism Conservation Center

Accession number: KCCM12628P

Date of deposit: 20191114

1. A microorganism comprising:
(a) a xylose assimilation enzyme; and
(b) a mycosporine-like amino acid biosynthesis enzyme,
wherein the xylose assimilation enzyme comprises a conversion enzyme of xylose to xylulose, a xylulose phosphorylase, or a combination thereof,
wherein the xylose assimilation enzyme, mycosporine-like amino acid biosynthesis enzyme, or both are exogenous proteins.

2. The microorganism according to claim 1, wherein
the conversion enzyme of xylose to xylulose is xylose isomerase (XI), xylose reductase (XR), xylitol dehydrogenase (XDH), or a combination thereof, and
the xylulose phosphorylase is xylulokinase (XK).

3. The microorganism according to claim 2, wherein
the xylose isomerase is derived from at least one selected from the group consisting of Pichia stipitis, Paraburkholderia sacchari, Actinomyces olivocinereus, Actinoplanes missouriensis, Aerobacter levanicum, Bacillus coagulans, Bacillus sp., Bifidobacterium adolescentis, Bacteroides thetaiotaomicron, Clostridium cellulovorans, Clostridium phytofermentans, Lactobacillus brevis, Lactococcus lactis, Orpinomyces sp., Piromyces sp., Thermus thermophiles, and Vibrio sp.,
the xylose reductase and the xylitol dehydrogenase are derived from at least one selected from the group consisting of Pichia sp., Aspergillus carbonarius, Candida sp., Kluyveromyces marxianus, Neurospora crassa, Ogataea siamensis, Trichoderma reesei, Zymomonas mobilis, Pachysolen tannophilus, Odontotaenius disjunctus), and Hansenula polymorpha,
the xylulokinase (XK) is derived from at least one selected from the group consisting of Pichia stipitis, Arabidopsis thaliana, Bacillus coagulans, Klebsiella pneumonia, Kluyveromyces marxianus, Hansenula polymorpha, Saccharomyces cerevisiae, Thermotoga maritime, Trichoderma reesei, and Zymomonas mobilis.

4. The microorganism according to claim 1, wherein the mycosporine-like amino acid biosynthesis enzyme comprises at least one selected from the consisting of:
2-dimethyl 4-deoxygadusol synthase (DDGS),
O-methyltransferase (O-MT),
C—N ligase or ATP-grasp ligase, and
non-ribosomal peptide synthetase, non-ribosomal peptide synthetase-like enzyme (NRPS-like enzyme), or D-alanine D-alanine ligase.

5. The microorganism according to claim 1, wherein the microorganism is one in which a pentose phosphate pathway is further enhanced.

6. The microorganism according to claim 5, wherein the enhancement of the pentose phosphate pathway is achieved through at least one selected from:
an increase in the transketolase activity to produce sedoheptulose 7-phosphate in the pentose phosphate pathway,
an increase in the protein activity that regulates NADPH production, and
a reduction in the transaldolase activity involved in the conversion reaction between sedoheptulose 7-phosphate and glyceraldehyde 3-phosphate.

7. The microorganism according to claim 1, wherein the microorganism is a yeast, a microorganism of the genus Corynebacterium, or a microorganism of the genus Escherichia.

8. The microorganism according to claim 1, wherein the microorganism is for producing mycosporine-like amino acids from xylose.

9. A composition for producing mycosporine-like amino acids, comprising the microorganism according to claim 1.

10. The composition for producing mycosporine-like amino acids according to claim 9, wherein the microorganism is a yeast, a microorganism of the genus Corynebacterium, or a microorganism of the genus Escherichia.

11. The composition for producing mycosporine-like amino acids according to claim 9, wherein the microorganism is for producing mycosporine-like amino acids from xylose.

12. The composition for producing mycosporine-like amino acids according to claim 9, wherein the mycosporine-like amino acid is at least one selected from the group consisting of mycosporine-2-glycine, palythinol, palythenic acid, deoxygadusol, mycosporine-methylaminethreonine, mycosporine-glycine-valine, palythine, asterina-330, shinorine, porphyra-334, euhalothece-362, mycosporine-glycine, mycosporine-ornithine, mycosporine-lysine, mycosporine-glutamic acid-glycine, mycosporine-methylamine-serine, mycosporine-taurine, palythene, palythine-serine, palythine-serine-sulfate, palythinol, and usujirene.

13. A method for producing mycosporine-like amino acids, comprising culturing the microorganism of claim 1.


SEQUENCE LISTING (69 sequences)

SEQ ID NO: 1 - DNA, 30 bp, Artificial Sequence
  Synthetic_NpR5600 F primer
gcgggatccatgagtaatgttcaagcatcg

SEQ ID NO: 2 - DNA, 29 bp, Artificial Sequence
  Synthetic_NpR5600 R primer
gcgctcgagtcacactcccaatagtttgg

SEQ ID NO: 3 - DNA, 29 bp, Artificial Sequence
  Synthetic_NpR5599 F primer
gcgggatccatgaccagtattttaggacg

SEQ ID NO: 4 - DNA, 30 bp, Artificial Sequence
  Synthetic_NpR5599 R primer
gcgctcgagttataccaagcgtctaatcag

SEQ ID NO: 5 - DNA, 30 bp, Artificial Sequence
  Synthetic_NpR5598 F primer
gcgggatccatggcacaatcaatctcttta

SEQ ID NO: 6 - DNA, 27 bp, Artificial Sequence
  Synthetic_NpR5598 R primer
gcgctcgagtagtcgccccctaattcc

SEQ ID NO: 7 - DNA, 30 bp, Artificial Sequence
  Synthetic_NpR5597 F primer
gcgggatccatgccagtacttaatatcctt

SEQ ID NO: 8 - DNA, 34 bp, Artificial Sequence
  Synthetic_NpR5597 R primer
gcgctcgagtcaattttgtaacacctttttatta

SEQ ID NO: 9 - DNA, 32 bp, Artificial Sequence
  Synthetic_XYL1 F primer
gcgggatccatgccttctattaagttgaactc

SEQ ID NO: 10 - DNA, 32 bp, Artificial Sequence
  Synthetic_XYL1 R primer
gcgctcgagttagacgaagataggaatcttgt

SEQ ID NO: 11 - DNA, 29 bp, Artificial Sequence
  Synthetic_XYL2 F primer
gcgggatccatgactgctaacccttcctt

SEQ ID NO: 12 - DNA, 28 bp, Artificial Sequence
  Synthetic_XYL2 R primer
gcgctcgagttactcagggccgtcaatg

SEQ ID NO: 13 - DNA, 28 bp, Artificial Sequence
  Synthetic_XYL3 F primer
gcgggatccatgaccactaccccatttg

SEQ ID NO: 14 - DNA, 34 bp, Artificial Sequence
  Synthetic_XYL3 R primer
gcgctcgagttagtgtttcaattcactttccatc

SEQ ID NO: 15 - DNA, 28 bp, Artificial Sequence
  Synthetic_STB5 F primer
gcgggatccatggatggtcccaattttg

SEQ ID NO: 16 - DNA, 32 bp, Artificial Sequence
  Synthetic_STB5 R primer
gcggtcgactcatacaagtttatcaacccaag

SEQ ID NO: 17 - DNA, 28 bp, Artificial Sequence
  Synthetic_TKL1 F primer
gcgggatccatgactcaattcactgaca

SEQ ID NO: 18 - DNA, 32 bp, Artificial Sequence
  Synthetic_TKL1 R primer
gcgctcgagttagaaagcttttttcaaaggag

SEQ ID NO: 19 - DNA, 38 bp, Artificial Sequence
  Synthetic_Delta1 R primer
atagcggccgcatgtttatattcattgatcctattaca

SEQ ID NO: 20 - DNA, 51 bp, Artificial Sequence
  Synthetic_Delta1 F primer
cacatttccccgaaaagtgcatttaaattgttggaatagaaatcaactatc

SEQ ID NO: 21 - DNA, 20 bp, Artificial Sequence
  Synthetic_Amp-Ori F primer
gcacttttcggggaaatgtg

SEQ ID NO: 22 - DNA, 50 bp, Artificial Sequence
  Synthetic_Amp-Ori R primer
ctcaacattcacccatttctcaatttaaatcgcaggaaagaacatgtgag

SEQ ID NO: 23 - DNA, 22 bp, Artificial Sequence
  Synthetic_Delta2 R primer
tgagaaatgggtgaatgttgag

SEQ ID NO: 24 - DNA, 32 bp, Artificial Sequence
  Synthetic_Delta2 F primer
gcggctagcataaaacggaatgaggaataatc

SEQ ID NO: 25 - DNA, 35 bp, Artificial Sequence
  Synthetic_Promoter up F primer
gcggctagcgagctcggaaacagctatgaccatga

SEQ ID NO: 26 - DNA, 38 bp, Artificial Sequence
  Synthetic_NTS term R primer
gcgggcgcgccgcggccgctaagggttctcgagagctc

SEQ ID NO: 27 - DNA, 43 bp, Artificial Sequence
  Synthetic_TEF prom F primer
gccaaaaatttacttcgcgcggcccgacatggaggcccagaat

SEQ ID NO: 28 - DNA, 19 bp, Artificial Sequence
  Synthetic_NTS1-2b F primer
gcgaagtaaatttttggcg

SEQ ID NO: 29 - DNA, 45 bp, Artificial Sequence
  Synthetic_NTS1-2b Amp R primer
tttccccgaaaagtgcatttaaatctagtttcttggcttcctatg

SEQ ID NO: 30 - DNA, 21 bp, Artificial Sequence
  Synthetic_NTS1-2a F primer
ccgagcgtgaaaggatttgcc

SEQ ID NO: 31 - DNA, 31 bp, Artificial Sequence
  Synthetic_NTS1-2a R primer
gcggctagccaaccattccatatctgttaag

SEQ ID NO: 32 - DNA, 31 bp, Artificial Sequence
  Synthetic_Univ F2 primer
gactcgcgcgcgggaacaaaagctggagctc

SEQ ID NO: 33 - DNA, 49 bp, Artificial Sequence
  Synthetic_Univ R primer
gactacgcgtgcggccgctaatggcgcgccatagggcgaattgggtacc

SEQ ID NO: 34 - DNA, 60 bp, Artificial Sequence
  Synthetic_TAL1_del F primer
tagtacgatagtaaaatacttctcgaactcgtcacatatacgtgtacataggaagtatct

SEQ ID NO: 35 - DNA, 60 bp, Artificial Sequence
  Synthetic_TAL1_del R primer
agaaacgtgcataaggacatggcctaaattaatatttccgagatacttcctatgtacacg

SEQ ID NO: 36 - DNA, 18 bp, Artificial Sequence
  Synthetic_TAL1_C F primer
cgggaataaagcggaact

SEQ ID NO: 37 - DNA, 18 bp, Artificial Sequence
  Synthetic_TAL1_C R primer
ggtggttccggatgtttt

SEQ ID NO: 38 - DNA, 40 bp, Artificial Sequence
  Synthetic_TAL1 gRNA F primer
aactaacccatcattgatctgttttagagctagaaatagc

SEQ ID NO: 39 - DNA, 40 bp, Artificial Sequence
  Synthetic_TAL1 gRNA R primer
agatcaatgatgggttagttgatcatttatctttcactgc

SEQ ID NO: 40 - DNA, 20 bp, Artificial Sequence
  Synthetic_NpR5600 qPCR F primer
atggcgaagagttgctttcc

SEQ ID NO: 41 - DNA, 20 bp, Artificial Sequence
  Synthetic_NpR5600 qPCR R primer
gtgtgaccgtaggcaatgac

SEQ ID NO: 42 - DNA, 20 bp, Artificial Sequence
  Synthetic_NpR5599 qPCR F primer
ccctcagaatggcagaggaa

SEQ ID NO: 43 - DNA, 20 bp, Artificial Sequence
  Synthetic_NpR5599 qPCR R primer
gacgcactgtttcaccatca

SEQ ID NO: 44 - DNA, 20 bp, Artificial Sequence
  Synthetic_NpR5598 qPCR F primer
ctagctgcacctttggaacc

SEQ ID NO: 45 - DNA, 20 bp, Artificial Sequence
  Synthetic_NpR5598 qPCR R primer
gagcgaatcccagtcaatcg

SEQ ID NO: 46 - DNA, 20 bp, Artificial Sequence
  Synthetic_NpR5597 qPCR F primer
aaactgacgatggcgacttg

SEQ ID NO: 47 - DNA, 20 bp, Artificial Sequence
  Synthetic_NpR5597 qPCR R primer
accaaggttgtccctttgga

SEQ ID NO: 48 - protein, 318 aa, Artificial Sequence
  Synthetic_xylose reductase
MPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPRLLENKDVNSFDLDEQDFADIAKLDINLRFNDPWDWDKIPIFV

SEQ ID NO: 49 - DNA, 957 bp, Artificial Sequence
  Synthetic_xylose reductase coding gene
atgccttctattaagttgaactctggttacgacatgccagccgtcggtttcggctgttggaaagtcgacgtcgacacctgttctgaacagatctaccgtgctatcaagaccggttacagattgttcgacggtgccgaagattacgccaacgaaaagttagttggtgccggtgtcaagaaggccattgacgaaggtatcgtcaagcgtgaagacttgttccttacctccaagttgtggaacaactaccaccacccagacaacgtcgaaaaggccttgaacagaaccctttctgacttgcaagttgactacgttgacttgttcttgatccacttcccagtcaccttcaagttcgttccattagaagaaaagtacccaccaggattctactgtggtaagggtgacaacttcgactacgaagatgttccaattttagagacctggaaggctcttgaaaagttggtcaaggccggtaagatcagatctatcggtgtttctaacttcccaggtgctttgctcttggacttgttgagaggtgctaccatcaagccatctgtcttgcaagttgaacaccacccatacttgcaacaaccaagattgatcgaattcgctcaatcccgtggtattgctgtcaccgcttactcttcgttcggtcctcaatctttcgttgaattgaaccaaggtagagctttgaacacttctccattgttcgagaacgaaactatcaaggctatcgctgctaagcacggtaagtctccagctcaagtcttgttgagatggtcttcccaaagaggcattgccatcattccaaagtccaacactgtcccaagattgttggaaaacaaggacgtcaacagcttcgacttggacgaacaagatttcgctgacattgccaagttggacatcaacttgagattcaacgacccatgggactgggacaagattcctatcttcgtctaa

SEQ ID NO: 50 - protein, 363 aa, Artificial Sequence
  Synthetic_xylitol dehydrogenase
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 51 - DNA, 1092 bp, Artificial Sequence
  Synthetic_xylitol dehydrogenase coding gene
atgactgctaacccttccttggtgttgaacaagatcgacgacatttcgttcgaaacttacgatgccccagaaatctctgaacctaccgatgtcctcgtccaggtcaagaaaaccggtatctgtggttccgacatccacttctacgcccatggtagaatcggtaacttcgttttgaccaagccaatggtcttgggtcacgaatccgccggtactgttgtccaggttggtaagggtgtcacctctcttaaggttggtgacaacgtcgctatcgaaccaggtattccatccagattctccgacgaatacaagagcggtcactacaacttgtgtcctcacatggccttcgccgctactcctaactccaaggaaggcgaaccaaacccaccaggtaccttatgtaagtacttcaagtcgccagaagacttcttggtcaagttgccagaccacgtcagcttggaactcggtgctcttgttgagccattgtctgttggtgtccacgcctctaagttgggttccgttgctttcggcgactacgttgccgtctttggtgctggtcctgttggtcttttggctgctgctgtcgccaagaccttcggtgctaagggtgtcatcgtcgttgacattttcgacaacaagttgaagatggccaaggacattggtgctgctactcacaccttcaactccaagaccggtggttctgaagaattgatcaaggctttcggtggtaacgtgccaaacgtcgttttggaatgtactggtgctgaaccttgtatcaagttgggtgttgacgccattgccccaggtggtcgtttcgttcaagtcggtaacgctgctggtccagtcagcttcccaatcaccgttttcgccatgaaggaattgactttgttcggttctttcagatacggattcaacgactacaagactgctgttggaatctttgacactaactaccaaaacggtagagaaaatgctccaattgactttgaacaattgatcacccacagatacaagttcaaggacgctattgaagcctacgacttggtcagagccggtaagggtgctgtcaagtgtctcattgacggccctgagtaa

SEQ ID NO: 52 - protein, 623 aa, Artificial Sequence
  Synthetic_xylulokinase
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLYTDGKKQTFESLTARPNRCYYVGGASNNGSIIRKMGSILAPVNGNYKVDIPNACALGGAYKASWSYECEAKKEWIGYDQYINRLFEVSDEMNLFEVKDKWLEYANGVGMLAKMESELKH

SEQ ID NO: 53 - DNA, 1872 bp, Artificial Sequence
  Synthetic_xylulokinase coding gene
atgaccactaccccatttgatgctccagataagctcttcctcgggttcgatctttcgactcagcagttgaagatcatcgtcaccgatgaaaacctcgctgctctcaaaacctacaatgtcgagttcgatagcatcaacagctctgtccagaagggtgtcattgctatcaacgacgaaatcagcaagggtgccattatttcccccgtttacatgtggttggatgcccttgaccatgtttttgaagacatgaagaaggacggattccccttcaacaaggttgttggtatttccggttcttgtcaacagcacggttcggtatactggtctagaacggccgagaaggtcttgtccgaattggacgctgaatcttcgttatcgagccagatgagatctgctttcaccttcaagcacgctccaaactggcaggatcactctaccggtaaagagcttgaagagttcgaaagagtgattggtgctgatgccttggctgatatctctggttccagagcccattacagattcacagggctccagattagaaagttgtctaccagattcaagcccgaaaagtacaacagaactgctcgtatctctttagtttcgtcatttgttgccagtgtgttgcttggtagaatcacctccattgaagaggccgatgcttgtggaatgaacttgtacgatatcgaaaagcgcgagttcaacgaagagctcttggccatcgctgctggtgtccaccctgagttggatggtgtagaacaagacggtgaaatttacagagctggtatcaatgagttgaagagaaagttgggtcctgtcaaacctataacatacgaaagcgaaggtgacattgcctcttactttgtcaccagatacggcttcaaccccgactgtaaaatctactcgttcaccggagacaatttggccacgattatctcgttgcctttggctccaaatgatgctttgatctcattgggtacttctactacagttttaattatcaccaagaactacgctccttcttctcaataccatttgtttaaacatccaaccatgcctgaccactacatgggcatgatctgctactgtaacggttccttggccagagaaaaggttagagacgaagtcaacgaaaagttcaatgtagaagacaagaagtcgtgggacaagttcaatgaaatcttggacaaatccacagacttcaacaacaagttgggtatttacttcccacttggcgaaattgtccctaatgccgctgctcagatcaagagatcggtgttgaacagcaagaacgaaattgtagacgttgagttgggcgacaagaactggcaacctgaagatgatgtttcttcaattgtagaatcacagactttgtcttgtagattgagaactggtccaatgttgagcaagagtggagattcttctgcttccagctctgcctcacctcaaccagaaggtgatggtacagatttgcacaaggtctaccaagacttggttaaaaagtttggtgacttgtacactgatggaaagaagcaaacctttgagtctttgaccgccagacctaaccgttgttactacgtcggtggtgcttccaacaacggcagcattatccgcaagatgggttccatcttggctcccgtcaacggaaactacaaggttgacattcctaacgcctgtgcattgggtggtgcttacaaggccagttggagttacgagtgtgaagccaagaaggaatggatcggatacgatcagtatatcaacagattgtttgaagtaagtgacgagatgaatctgttcgaagtcaaggataaatggctcgaatatgccaacggggttggaatgttggccaagatggaaagtgaattgaaacactaa

SEQ ID NO: 54 - protein, 680 aa, Artificial Sequence
  Synthetic_TKL1
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 55 - DNA, 2043 bp, Artificial Sequence
  Synthetic_TKL1 coding gene
atgactcaattcactgacattgataagctagccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaa

SEQ ID NO: 56 - protein, 743 aa, Artificial Sequence
  Synthetic_Stb5
MDGPNFAHQGGRSQRTTELYSCARCRKLKKKCGKQIPTCANCDKNGAHCSYPGRAPRRTKKELADAMLRGEYVPVKRNKKVGKSPLSTKSMPNSSSPLSANGAITPGFSPYENDDAHKMKQLKPSDPINLVMGASPNSSEGVSSLISVLTSLNDNSNPSSHLSSNENSMIPSRSLPASVQQSSTTSSFGGYNTPSPLISSHVPANAQAVPLQNNNRNTSNGDNGSNVNHDNNNGSTNTPQLSLTPYANNSAPNGKFDSVPVDASSIEFETMSCCFKGGRTTSWVREDGSFKSIDRSLLDRFIAAYFKHNHRLFPMIDKIAFLNDAATITDFERLYDNKNYPDSFVFKVYMIMAIGCTTLQRAGMVSQDEECLSEHLAFLAMKKFRSVIILQDIETVRCLLLLGIYSFFEPKGSSSWTISGIIMRLTIGLGLNRELTAKKLKSMSALEAEARYRVFWSAYCFERLVCTSLGRISGIDDEDITVPLPRALYVDERDDLEMTKLMISLRKMGGRIYKQVHSVSAGRQKLTIEQKQEIISGLRKELDEIYSRESERRKLKKSQMDQVERENNSTTNVISFHSSEIWLAMRYSQLQILLYRPSALMPKPPIDSLSTLGEFCLQAWKHTYTLYKKRLLPLNWITLFRTLTICNTILYCLCQWSIDLIESKIEIQQCVEILRHFGERWIFAMRCADVFQNISNTILDISLSHGKVPNMDQLTRELFGASDSYQDILDENNVDVSWVDKLV

SEQ ID NO: 57 - DNA, 2232 bp, Artificial Sequence
  Synthetic_Stb5 gene
atggatggtcccaattttgcacatcaaggcgggagatcacaacgtactactgaattgtattcgtgcgcacgatgcagaaaattaaagaagaagtgtggtaaacaaataccgacatgtgcaaactgcgataaaaatggggcacactgttcatatccaggtagagccccaagacgtaccaagaaggagttagcggatgctatgctacgaggggaatatgttccagtgaaaaggaacaagaaggtaggaaaaagcccattgagcactaagagcatgccaaactcttctagtccgctatccgcaaatggcgctataactcccgggttttcgccttacgaaaacgatgatgcacataagatgaaacagctaaaaccgtcagatccaataaatcttgtcatgggggcaagtccaaattctagcgaaggtgtctcatcgctaatttcggtgctaacatcgctgaatgataattctaatccttcttcgcacttatcctctaatgaaaattccatgattccttcccgatcattgccagcttccgtgcagcaaagttcgacaacttcatcattcggaggatataacacgccttcaccactaattagcagtcatgtgcctgcgaacgcccaagccgtaccgctacaaaacaacaatcgcaatactagcaacggggataacggcagtaatgttaaccacgataataacaatggcagtaccaacacaccgcaattgagtcttaccccatatgcaaacaattcagcccccaatgggaaattcgattctgtgccggttgatgcatcctcgatcgaatttgaaactatgtcctgttgctttaaaggtggtagaacaacatcgtgggtcagagaggatggctcgttcaagtcaattgatagatccttactggacaggttcattgccgcatacttcaaacacaatcaccgtctatttcccatgattgataaaatagcattcctaaatgacgccgcgacaattactgatttcgaaaggttatatgacaacaaaaactaccctgacagctttgttttcaaagtatacatgatcatggctattggttgtacaactttacagcgtgctggtatggtttctcaggacgaagagtgtctgagtgaacatttggcttttttggccatgaaaaaatttcgtagtgttataattttacaagatatcgaaactgtacgatgcctattgttgttgggtatttattcgttttttgagccaaagggctcctcgtcatggacaattagtggtatcatcatgcgattgaccataggattaggtttaaatagagagttaactgccaaaaaactcaagagcatgtctgctttagaagcagaggcaagatatagagtgttttggagtgcttactgctttgaaaggctagtatgcacctcgttgggccgtatatccgggatcgacgacgaagacatcactgtgccactaccgagggcgttgtatgtggatgaaagagacgatttagagatgaccaagttgatgatatcattaaggaaaatgggcggtcgcatttataaacaagtccactctgtaagtgcagggcgacaaaagttaaccatcgaacaaaagcaggaaatcataagtggattacgcaaggagctagacgaaatttattctcgagaatcagaaagaaggaaactgaaaaaatctcaaatggatcaggtggagagggagaacaattctactacaaatgtaatatccttccatagttctgagatttggctagcaatgagatactcccagttgcaaatcttactatacagaccatctgcattgatgccaaaaccgcccattgactcactatccactctaggagagttttgcttgcaagcctggaaacatacttatacactgtacaagaagcggttattacccttgaactggataacccttttcagaacattaaccatttgtaacactatcttatactgtctttgccagtggagcatcgacctcattgaaagtaaaatcgaaattcaacagtgtgtggaaatactaagacatttcggtgaaagatggatttttgctatgagatgcgcggatgttttccaaaacattagcaacacaattctcgatattagtttaagccatggtaaagttcctaatatggaccaattaacaagagagttatttggcgccagcgattcatatcaagacatattagacgaaaacaatgttgacgtctcttgggttgataaacttgtatga

SEQ ID NO: 58 - protein, 335 aa, Artificial Sequence
  Synthetic_TAL1
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 59 - DNA, 1008 bp, Artificial Sequence
  Synthetic_TAL1 coding gene
atgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttggtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaattactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaa

SEQ ID NO: 60 - protein, 410 aa, Artificial Sequence
  Synthetic_DDGS
MSNVQASFEATEAEFRVEGYEKIEFSLVYVNGAFDISNREIADSYEKFGRCLTVIDANVNRLYGKQIKSYFRHYGIDLTVVPIVITEPTKTLATFEKIVDAFSDFGLIRKEPVLVVGGGLTTDVAGFACAAYRRKSNYIRVPTTLIGLIDAGVAIKVAVNHRKLKNRLGAYHAPLKVILDFSFLQTLPTAQVRNGMAELVKIAVVANSEVFELLYEYGEELLSTHFGYVNGTKELKAIAHKLNYEAIKTMLELETPNLHELDLDRVIAYGHTWSPTLELAPMIPLFHGHAVNIDMALSATIAARRGYITSGERDRILSLMSRIGLSIDHPLLDGDLLWYATQSISLTRDGKQRAAMPKPIGECFFVNDFTREELDAALAEHKRLCATYPRGGDGIDAYIETQEESKLLGV

SEQ ID NO: 61 - DNA, 1233 bp, Artificial Sequence
  Synthetic_DDGS coding gene
atgagtaatgttcaagcatcgtttgaagcaacggaagctgaattccgcgtggaaggttacgaaaaaattgagtttagtcttgtctatgtaaatggtgcatttgatatcagtaacagagaaattgcagacagctatgagaagtttggccgttgtctgactgtgattgatgctaatgtcaacagattatatggcaagcaaatcaagtcatattttagacactatggtattgatctgaccgtagttcccattgtgattactgagcctactaaaacccttgcaacctttgagaaaattgttgatgctttttctgactttggtttaatccgcaaggaaccagtattagtagtgggtggtggtttaaccactgatgtagctgggtttgcgtgtgctgcttaccgtcgtaagagtaactatattcgggttccgacaacgctgattggtctgattgatgcaggtgtagcgattaaggttgcagtcaaccatcgcaagttaaaaaatcgcttgggtgcatatcatgctcctttgaaagtcatcctcgatttctccttcttgcaaacattaccaacggctcaagttcgtaatgggatggcagagttggtcaaaattgctgttgtggcgaactctgaagtctttgaattgttgtatgaatatggcgaagagttgctttccactcactttggatatgtgaatggtacaaaggaactgaaagcgatcgcacataaactcaattacgaggcaataaaaactatgctggagttggaaactccaaacttgcatgagttagacctcgatcgcgtcattgcctacggtcacacttggagtccgaccttagaattagctccgatgataccgttgtttcacggtcatgccgtcaatatagacatggctttgtctgcaactattgcggcaagacgtggctacattacatcaggagaacgcgatcgcattttgagcttgatgagtcgtataggtttatcaatcgatcatcctctactagatggcgatttgctctggtatgctacccaatctattagcttgacgcgagacgggaaacaacgcgcagctatgcctaaacccattggtgagtgcttctttgtcaacgatttcacccgtgaagaactagatgcagctttagctgaacacaaacgtctgtgtgctacataccctcgtggtggagatggcattgacgcttacatagaaactcaagaagaatccaaactattgggagtgtga

SEQ ID NO: 62 - protein, 277 aa, Artificial Sequence
  Synthetic_O-MT
MTSILGRDTARPITPHSILVAQLQKTLRMAEESNIPSEILTSLRQGLQLAAGLDPYLDDCTTPESTALTALAQKTSIEDWSKRFSDGETVRQLEQEMLSGHLEGQTLKMFVHITKAKSILEVGMFTGYSALAMAEALPDDGRLIACEVDSYVAEFAQTCFQESPHGRKIVVEVAPALETLHKLVAKKESFDLIFIDADKKEYIEYFQIILDSHLLAPDGLICVDNTLLQGQVYLPSEQRTANGEAIAQFNRIVAADPRVEQVLLPIRDGITLIRRLV

SEQ ID NO: 63 - DNA, 834 bp, Artificial Sequence
  Synthetic_O-MT coding gene
atgaccagtattttaggacgagataccgcaagaccaataacgccacatagcattctggtagcacagctacaaaaaaccctcagaatggcagaggaaagtaatattccttcagagatactgacttctctgcgccaagggttgcaattagcagcaggtttagatccctatctggatgattgcactacccctgaatcgaccgcattgacagcactagcgcagaagacaagcattgaagactggagtaaacgcttcagtgatggtgaaacagtgcgtcaattagagcaagaaatgctctcaggacatcttgaaggacaaacactaaagatgtttgtgcatatcactaaggctaaaagcatcctagaagtgggaatgttcacaggatattcagctttggcaatggcagaggcgttaccagatgatgggcgactgattgcttgtgaagtagactcctatgtggccgagtttgctcaaacttgctttcaagagtctccccacggccgcaagattgttgtagaagtggcacctgcactagagacgctgcacaagctggtggctaaaaaagaatcctttgatctgatcttcattgatgcggataaaaaggagtatatagaatacttccaaattatcttggatagccatttactagctcccgacggattaatctgtgtagataatactttgttgcaaggacaagtttacctgccatcagaacagcgtacagccaatggtgaagcgatcgctcaattcaaccgcattgtcgccgcagatcctcgtgtagagcaagttctgttacccatacgagatggtataaccctgattagacgcttggtataa

SEQ ID NO: 64 - protein, 467 aa, Artificial Sequence
  Synthetic_ATP-grasp ligase
MAQSISLSLPQSTTPSKGVRLKIAALLKTIGTLILLLIALPLNALIVLISLMCRPFTKKPAVATHPQNILVSGGKMTKALQLARSFHAAGHRVILIEGHKYWLSGHRFSNSVSRFYTVPAPQDDPEGYTQALLEIVKREKIDVYVPVCSPVASYYDSLAKSALSEYCEVFHFDADITKMLDDKFAFTDRARSLGLSAPKSFKITDPEQVINFDFSKETRKYILKSISYDSVRRLNLTKLPCDTPEETAAFVKSLPISPEKPWIMQEFIPGKELCTHSTVRDGELRLHCCSNSSAFQINYENVENPQIQEWVQHFVKSLRLTGQISLDFIQAEDGTAYAIECNPRTHSAITMFYNHPGVAEAYLGKTPLAAPLEPLADSKPTYWIYHEIWRLTGIRSGQQLQTWFGRLVRGTDAIYRLDDPIPFLTLHHWQITLLLLQNLQRLKGWVKIDFNIGKLVELGGDYSRSEE

SEQ ID NO: 65 - DNA, 1404 bp, Artificial Sequence
  Synthetic_ATP-grasp ligase coding gene
atggcacaatcaatctctttatctcttcctcaatccacaacgccatcaaagggtgtgaggctaaaaatagcagctctactgaagactatcgggactctaatactactgctgatagccttgccgcttaatgctttgatagtattgatatctctgatgtgtaggccgtttacaaaaaaacctgccgtagccactcatccccagaatatcttggtcagtggcggcaaaatgaccaaagcattgcaacttgcccgctccttccatgcagccggacacagagttattctgattgaaggtcataaatactggttatcagggcatagattctcaaattctgtgagtcgtttttatacagttcctgcaccacaagacgacccagaaggctatacccaagcgctattggaaattgtcaaacgagagaagattgacgtttatgtacccgtatgcagccctgtagctagttattacgactctttggcaaagtctgcactatcagaatattgtgaggtttttcactttgatgctgatataaccaagatgctggatgataaatttgcctttaccgatcgggcgcgatcgcttggtttatcagccccgaaatcttttaaaattaccgatcctgaacaagttatcaacttcgattttagtaaagagacgcgcaaatatattcttaagagtatttcttacgactcagttcgccgcttaaatttaaccaaacttccttgtgataccccagaagagacagcagcgtttgtcaagagtttacccatcagcccagaaaaaccttggattatgcaagaatttattcctgggaaagaattatgcacccatagcacagtccgagacggcgaattaaggttgcattgctgttcaaattcttcagcgtttcagattaactatgaaaatgtcgaaaatccccaaattcaagaatgggtacaacatttcgtcaaaagtttacggctgactggacaaatatctcttgactttatccaagctgaagatggtacagcttatgccattgaatgtaatcctcgcacccattcggcgatcacaatgttctacaatcacccaggtgttgcagaagcctatcttggtaaaactcctctagctgcacctttggaacctcttgcagatagcaagcccacttactggatatatcacgaaatctggcgattgactgggattcgctctggacaacaattacaaacttggtttgggagattagtcagaggtacagatgccatttatcgcctggacgatccgataccatttttaactttgcaccattggcagattactttacttttgctacaaaatttgcaacgactcaaaggttgggtaaagatcgattttaatatcggtaaactcgtggaattagggggcgactactcgaggtctgaagaatga

SEQ ID NO: 66 - protein, 348 aa, Artificial Sequence
  Synthetic_D-ala-D-ala ligase
MPVLNILHLVGSAHDKFYCDLSRLYAQDCLAATADPSLYNFQIAYITPDRQWRFPDSLSREDIALTKPIPVFDAIQFLTGQNIDMMLPQMFCIPGMTQYRALFDLLKIPYIGNTPDIMAIAAHKARAKAIVEAAGVKVPRGELLRQGDIPTITPPAVVKPVSSDNSLGVVLVKDVTEYDAALKKAFEYASEVIVEAFIELGREVRCGIIVKDGELIGLPLEEYLVDPHDKPIRNYADKLQQTDDGDLHLTAKDNIKAWILDPNDPITQKVQQVAKRCHQALGCRHYSLFDFRIDPKGQPWFLEAGLYCSFAPKSVISSMAKAAGIPLNDLLITAINETLGSNKKVLQN

SEQ ID NO: 67 - DNA, 1047 bp, Artificial Sequence
  Synthetic_D-ala-D-ala ligase coding gene
atgccagtacttaatatccttcatttagttgggtctgcacacgataagttttactgtgatttatcacgtctttatgcccaagactgtttagctgcaacagcagatccatcgctttataactttcaaattgcatatatcacacccgatcggcagtggcgatttcctgactctctcagtcgagaagatattgctcttaccaaaccgattcctgtgtttgatgccatacaatttctaacaggccaaaacattgacatgatgttaccacaaatgttttgtattcctggaatgactcagtaccgtgccctattcgatctgctcaagatcccttatataggaaataccccagatattatggcgatcgcggcccacaaagccagagccaaagcaattgtcgaagcagcaggggtaaaagtgcctcgtggagaattgcttcgccaaggagatattccaacaattacacctccagcagtcgtcaaacctgtaagttctgacaactctttaggagtagtcttagttaaagatgtgactgaatatgatgctgccttaaagaaagcatttgaatatgcttcggaggtcatcgtagaagcattcatcgaacttggtcgagaagtcagatgcggcatcattgtaaaagacggtgagctaataggtttaccccttgaagagtatctggtagacccacacgataaacctatccgtaactatgctgataaactccaacaaactgacgatggcgacttgcatttgactgctaaagataatatcaaggcttggattttagaccctaacgacccaatcacccaaaaggttcagcaagtggctaaaaggtgtcatcaggctttgggttgtcgccactacagtttatttgacttccgaatcgatccaaagggacaaccttggttcttagaagctggattatattgttcttttgcccccaaaagtgtgatttcttctatggcgaaagcagccggaatccctctaaatgatttattaataaccgctattaatgaaacattgggtagtaataaaaaggtgttacaaaattga

SEQ ID NO: 68 - protein, 888 aa, Artificial Sequence
  Synthetic_NRPS
MQTIDFNIRKLLVEWNATHRDYDLSQSLHELIVAQVERTPEAIAVTFDKQQLTYQELNHKANQLGHYLQTLGVQPETLVGVCLERSLEMVICLLGILKAGGAYVPIDPEYPQERIAYMLEDSQVKVLLTQEKLLNQIPHHQAQTICVDREWEKISTQANTNPKSNIKTDNLAYVIYTSGSTGKPKGAMNTHKGICNRLLWMQEAYQIDSTDSILQKTPFSFDVSVWEFFWTLLTGARLVIAKPGGHKDSAYLIDLITQEQITTLHFVPSMLQVFLQNRHVSKCSSLKRVICSGEALSIDLQNRFFQHLQCELHNLYGPTEAAIDVTFWQCRKDSNLKSVPIGRPIANTQIYILDADLQPVNIGVTGEIYIGGVGVARGYLNKEELTKEKFIINPFPNSEFKRLYKTGDLARYLPDGNIEYLGRTDYQVKIRGYRIEIGEIENVLSSHPQVREAVVIARDDNAQEKQIIAYITYNSIKPQLDNLRDFLKARLPDFMIPAAFVMLEHLPLTPSGKVDRKALPKPDLFNYSEHNSYVAPRNEVEEKLVQIWSNILHLPKVGVTENFFAIGGNSLKALHLISQIEELFAKEISLATLLTNPVIADLAKVIQANNQIHNSPLVPIQPQGKQQPFFCIHPAGGHVLCYFKLAQYIGTDQPFYGLQAQGFYGDEAPLTRVEDMASLYVKTIREFQPQGPYRVGGWSFGGVVAYEVAQQLHRQGQEVSLLAILDSYVPILLDKQKPIDDVYLVGVLSRVFGGMFGQDNLVTPEEIENLTVEEKINYIIDKARSARIFPPGVERQNNRRILDVLVGTLKATYSYIRQPYPGKVTVFRAREKHIMAPDPTLVWVELFSVMAAQEIKIIDVPGNHYSFVLEPHVQVLAQRLQDCLENNS

SEQ ID NO: 69 - DNA, 2667 bp, Artificial Sequence
  Synthetic_NRPS coding gene
atgcagactatagattttaatattcgtaagttacttgtagagtggaacgcgacccacagagattatgatctttcccagagtttacatgaactaattgtagctcaagtagaacgaacacctgaggcgatcgctgtcacctttgacaagcaacaactaacttatcaagaactaaatcataaagcaaaccagctaggacattatttacaaacattaggagtccagccagaaaccctggtaggcgtttgtttagaacgttccttagaaatggttatctgtcttttaggaatcctcaaagctgggggtgcttatgttcctattgaccctgaatatcctcaagaacgcatagcttatatgctagaagattctcaggtgaaggtactactaactcaagaaaaattactcaatcaaattccccaccatcaagcacaaactatctgtgtagatagggaatgggagaaaatttccacacaagctaataccaatcccaaaagtaatataaaaacggataatcttgcttatgtaatttacacctctggttccactggtaaaccaaaaggtgcaatgaacacccacaaaggtatctgtaatcgcttattgtggatgcaggaagcttatcaaatcgattccacagatagcattttacaaaaaaccccctttagttttgatgtttccgtttgggagttcttttggactttattaactggcgcacgtttggtaatagccaaaccaggcggacataaagatagtgcttacctcatcgatttaattactcaagaacaaatcactacgttgcattttgtcccctcaatgctgcaagtgtttttacaaaatcgccatgtaagcaaatgcagctctctaaaaagagttatttgtagcggtgaagctttatctatagatttacaaaatagatttttccagcatttgcaatgtgaattacataacctctatggcccgacagaagcagcaattgatgtcacattttggcaatgtagaaaagatagtaatttaaagagtgtacctattggtcgtcccattgctaatactcaaatttatattcttgatgccgatttacaaccagtaaatattggtgtcactggtgaaatttatattggtggtgtaggggttgctcgtggttatttgaataaagaagaattgaccaaagaaaaatttattattaatccctttcccaattctgagtttaagcgactttataaaacaggtgatttagctcgttatttacccgatggaaatattgaatatcttggtagaacagattatcaagtaaaaattcggggttatagaattgaaattggcgagattgaaaatgttttatcttcacacccacaagtcagagaagctgtagtcatagcgcgggatgataacgctcaagaaaaacaaatcatcgcttatattacctataactccatcaaacctcagcttgataatctgcgtgatttcctaaaagcaaggctacctgattttatgattccagccgcttttgtgatgctggagcatcttcctttaactcccagtggtaaagtagaccgtaaggcattacctaagcctgatttatttaattatagtgaacataattcctatgtagcgcctcggaatgaagttgaagaaaaattagtacaaatctggtcgaatattctgcatttacctaaagtaggtgtgacagaaaactttttcgctattggtggtaattccctcaaagctctacatttaatttctcaaattgaagagttatttgctaaagagatatccttagcaacacttttaacaaatccagtaattgcagatttagccaaggttattcaagcaaacaaccaaatccataattcacccctagttccaattcaaccacaaggtaagcagcagcctttcttttgtatacatcctgctggtggtcatgttttatgctattttaaactcgcacaatatataggaactgaccaaccattttatggcttacaagctcaaggattttatggagatgaagcacccttgacgcgagttgaagatatggctagtctctacgtcaaaactattagagaatttcaaccccaagggccttatcgtgtcggggggtggtcatttggtggagtcgtagcttatgaagtagcacagcagttacatagacaaggacaagaagtatctttactagcaatattagattcttacgtaccgattctgctggataaacaaaaacccattgatgacgtttatttagttggtgttctctccagagtttttggcggtatgtttggtcaagataatctagtcacacctgaagaaatagaaaatttaactgtagaagaaaaaattaattacatcattgataaagcacggagcgctagaatattcccgcctggtgtagaacgtcaaaataatcgccgtattcttgatgttttggtgggaactttaaaagcaacttattcctatataagacaaccatatccaggaaaagtcactgtatttcgagccagggaaaaacatattatggctcctgacccgaccttagtttgggtagaattattttctgtaatggcggctcaagaaattaagattattgatgtccctggaaaccattattcgtttgttctagaaccccatgtacaggttttagcacagcgtttacaagattgtctggaaaataattcataa